(19) United States
(12) Patent Application Publication
(10) Pub. No.: US 2023/0340409 A1
(43) Pub. Date: Oct. 26, 2023

(54) ENGINEERED IMMUNE CELLS WITH PRIMING RECEPTORS

(71) Applicant: Arsenal Biosciences, Inc., South San Francisco, CA (US)

(72) Inventors: Aaron Cooper, Berkeley, CA (US); Michelle Nguyen, San Francisco, CA (US); Anzhi Yao, San Francisco, CA (US); Stephen Santoro, Daly City, CA (US)

(21) Appl. No.: 18/114,981

(22) Filed: Feb. 27, 2023

Related U.S. Application Data
(63) Continuation of application No. PCT/US2021/048066, filed on Aug. 27, 2021.
(60) Provisional application No. 63/072,080, filed on Aug. 28, 2020.

(51) Int. Cl. C12N 5/078; C07K 14/725; A61P 35/00

(52) U.S. Cl. CPC C12N 5/0634; C07K 14/7051; A61P 35/00; C07K 2319/03

(57) ABSTRACT

Provided herein are methods of genetically editing cells with large DNA templates using a non-viral editing method. Also provided herein are cells comprising at least one large DNA template non-virally inserted into a target region of the genome of the cell.

### CROSS REFERENCE TO RELATED APPLICATIONS

This application is a Continuation of International Application No. PCT/US2021/048066, filed Aug. 27, 2021, which claims the benefit of U.S. Provisional Application No. 63/072,080, filed Aug. 28, 2020, which is hereby incorporated in its entirety by reference.

### SEQUENCE LISTING

The instant application contains a Sequence Listing which has been submitted via Patent Center and is hereby incorporated by reference in its entirety. Said XML copy, created on May 3, 2023, is named ANB-209WOC1_SL.xml, and is 183,204 bytes in size.

### BACKGROUND

Cancer is a disease characterized by uncontrollable growth of cells. Many approaches to treating cancer have been tried, including drugs and radiation therapies. Recent cancer treatments have sought to use the body's own immune cells to attack cancer cells. One promising approach uses T cells that are taken from a patient and genetically engineered to produce chimeric antigen receptors, or CARs, receptor proteins that give the T cells a new ability to target a specific protein. The receptors are chimeric because they combine antigen-binding and T-cell activating functions into a single receptor.

Immunotherapy using those CAR-T cells is promising because the modified T cells have the potential to recognize cancer cells in order to more effectively target and destroy them.

After the T cells are engineered with the CARs, the resulting CAR-T cells are introduced into patients to attack tumor cells. CAR-T cells can be either derived from T cells in a patient's own blood (autologous) or derived from the T cells of another healthy donor (allogeneic).

Once CAR-T cells are infused into a patient, they come in contact with their targeted antigen on a cell. The CAR-T cells bind to the antigen and become activated. Upon antigen engagement, CAR T cells can proliferate exponentially, initiate antitumor cytokine production, and target tumor cell killing. However, there remain some concerns and limitations to CAR T cell-based immunotherapy. Some CAR T cells may engage with normal cells expressing low levels of target antigens, leading to off target toxicity.

Furthermore, insertion of large gene constructs into specific gene targets is difficult using viral vectors, a known bottleneck in immune cell engineering.

### SUMMARY

In one aspect, provided herein are primary immune cells comprising at least one DNA template non-virally inserted into a target region of the genome of the cell, wherein the size of the DNA template is greater than or equal to about 5 kilobase pairs (kb).

In some embodiments, the primary immune cell does not comprise a viral vector for introducing the DNA template into the primary immune cell.

In some embodiments, the size of the DNA template is greater than or equal to about 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb. 9.9 kb, 10.0 kb or any size of DNA template in between these sizes.

In some embodiments, the size of the DNA template is about 5 kb to about 10 kb, about 5 kb to about 9 kb, about 5 kb to about 8 kb, about 5 kb to about 7 kb, about 5 kb to about 6 kb, about kb 6 to about 10 kb, about 6 kb to about 9 kb, about 6 kb to about 8 kb, about 6 kb to about 7 kb, about 7 kb to about 10 kb, about 7 kb to about 9 kb, about 7 kb to about 8 kb, about 8 kb to about 10 kb, about 8 kb to about 9 kb, or about 9 kb to about 10 kb.

In some embodiments, the DNA template is a double-stranded DNA template or a single-stranded DNA template.

In some embodiments, the DNA template is a linear DNA template or a circular DNA template, optionally wherein the circular DNA template is a plasmid.

In some embodiments, the 5′ and 3′ ends of the DNA template comprise nucleotide sequences that are homologous to genomic sequences flanking an insertion site in the genome of the primary cell.

In some embodiments, the target region of the genome of the cell is a T Cell Receptor Alpha Constant (TRAC) locus or a genomic safe harbor (GSH).

In some embodiments, the DNA template comprises a heterologous sequence.

In some embodiments, the DNA template comprises a gene.

In some embodiments, the DNA template comprises a priming receptor comprising a transcription factor.

In some embodiments, the DNA template comprises a chimeric antigen receptor (CAR).

In some embodiments, the DNA template comprises a chimeric antigen receptor (CAR) and a priming receptor comprising a transcription factor.

In some embodiments, the DNA template comprises an inducible promoter operably linked to the chimeric antigen receptor.

In some embodiments, the DNA template further comprises a constitutive promoter operably linked to the priming receptor.

In some embodiments, the DNA template further comprises an inducible promoter operably linked to the chimeric antigen receptor and a constitutive promoter operably linked to the priming receptor.

In some embodiments, the DNA template comprises, in a 5′ to 3′ direction: the inducible promoter; the chimeric antigen receptor, the constitutive promoter; and the priming receptor.

In some embodiments, the DNA template comprises, in a 5′ to 3′ direction: the constitutive promoter; the priming receptor, the inducible promoter; and the chimeric antigen receptor.

In some embodiments, the DNA template further comprises a self-excising 2A peptide (P2A).

In some embodiments, the P2A nucleic acid is at the 3′ end of the DNA template.

In some embodiments, the DNA template further comprises a woodchuck hepatitis virus post-translational regulatory element (WPRE).

In some embodiments, the WPRE is at the 3′ end of the nucleic acid encoding the CAR and at the 5′ end of the nucleic acid encoding the priming receptor or wherein the WPRE is at the 3′ end of the nucleic acid encoding the priming receptor and at the 5′ end of the nucleic acid encoding the CAR.

In some embodiments, the priming receptor comprises, in an N terminus to C terminus direction: an extracellular antigen-binding domain having a binding affinity for an antigen; a transmembrane domain comprising one or more ligand-inducible proteolytic cleavage sites; and an intracellular domain comprising a human or humanized transcriptional effector, wherein binding of an antigen to the extracellular antigen-binding domain results in cleavage at the ligand-inducible proteolytic cleavage site thereby releasing the intracellular domain.

In some embodiments, the priming receptor further comprises a juxtamembrane domain (JMD) positioned between the transmembrane domain and the intracellular domain.

In some embodiments, the transcription factor binds to the inducible promoter and induces expression of the CAR.

In some embodiments, the CAR comprises, from N-terminus to C-terminus, an extracellular antigen-binding domain having a binding affinity for an antigen; a transmembrane domain; an intracellular co-stimulatory domain; and an intracellular activation domain.

In some embodiments, the priming receptor and the CAR bind different antigens.

In some embodiments, the priming receptor and the CAR bind the same antigen.

In some embodiments, the immune cell is a primary human immune cell.

In some embodiments, the primary immune cell is an autologous immune cell.

In some embodiments, the primary immune cell is a natural killer (NK) cell, a T cell, a CD8+ T cell, a CD4+ T cell, a primary T cell, or a T cell progenitor.

In some embodiments, the primary immune cell is a primary T cell.

In some embodiments, the primary immune cell is a primary human T cell.

In some embodiments, the primary immune cell is virus-free.

In another aspect, provided herein are populations of cells comprising a plurality of the primary immune cell disclosed herein.

In another aspect, provided herein are primary immune cells comprising at least one DNA template inserted into a target region of the genome of the primary immune cell, wherein the size of the DNA template is greater than or equal to 5 kilobase pairs, and wherein the primary immune cell does not comprise a viral vector for introducing the DNA template into the primary immune cell.

In another aspect, provided herein are primary immune cells comprising at least one DNA template comprising a chimeric antigen receptor (CAR) and a priming receptor comprising a transcription factor inserted into a target region of the genome of the primary immune cell, wherein the size of the DNA template is greater than or equal to 5 kilobase pairs, and wherein the primary immune cell does not comprise a viral vector for introducing the DNA template into the primary immune cell.

In another aspect, provided herein are viable, virus-free, primary cells comprising a ribonucleoprotein complex (RNP)-DNA template complex, wherein the RNP comprises a nuclease domain and a guide RNA, wherein the size of the DNA template is greater than or equal to 5 kilobase nucleotides in size, and wherein the 5′ and 3′ ends of the DNA template comprise nucleotide sequences that are homologous to genomic sequences flanking an insertion site in the genome of the primary cell.

In another aspect, provided herein are viable, virus-free, primary cells comprising a ribonucleoprotein complex (RNP)-DNA template complex, wherein the RNP comprises a nuclease domain and a guide RNA, wherein the size of the DNA template is greater than or equal to 5 kilobase nucleotides in size, wherein the DNA template comprises a chimeric antigen receptor (CAR) and a priming receptor comprising a transcription factor, and wherein the 5′ and 3′ ends of the DNA template comprise nucleotide sequences that are homologous to genomic sequences flanking an insertion site in the genome of the primary cell.

In another aspect, provided herein are methods of treating a disease in a subject comprising administering the primary immune cell to the subject.

In some embodiments, the disease is cancer.

In another aspect, provided herein are non-viral vectors comprising a DNA template, wherein the DNA template is greater than or equal to 5 kilobase nucleotides in size, and wherein the 5′ and 3′ ends of the DNA template comprise nucleotide sequences that are homologous to genomic sequences flanking an insertion site in a genome of a primary cell.

In some embodiments, the size of the DNA template is greater than or equal to about 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb. 9.9 kb, 10.0 kb or any size of DNA template in between these sizes.

In some embodiments, the size of the DNA template is about 5 kb to about 10 kb, about 5 kb to about 9 kb, about 5 kb to about 8 kb, about 5 kb to about 7 kb, about 5 kb to about 6 kb, about kb 6 to about 10 kb, about 6 kb to about 9 kb, about 6 kb to about 8 kb, about 6 kb to about 7 kb, about 7 kb to about 10 kb, about 7 kb to about 9 kb, about 7 kb to about 8 kb, about 8 kb to about 10 kb, about 8 kb to about 9 kb, or about 9 kb to about 10 kb.

In some embodiments, the target region of the genome of the cell is a T Cell Receptor Alpha Constant (TRAC) locus or a genomic safe harbor (GSH).

In some embodiments, the DNA template comprises a heterologous sequence.

In some embodiments, the DNA template comprises a gene.

In some embodiments, the DNA template comprises a priming receptor comprising a transcription factor.

In some embodiments, the DNA template comprises a chimeric antigen receptor (CAR).

In some embodiments, the DNA template comprises a chimeric antigen receptor (CAR) and a priming receptor comprising a transcription factor.

In some embodiments, the DNA template comprises an inducible promoter operably linked to the chimeric antigen receptor.

In some embodiments, the DNA template further comprises a constitutive promoter operably linked to the priming receptor.

In some embodiments, the DNA template further comprises an inducible promoter operably linked to the chimeric antigen receptor and a constitutive promoter operably linked to the priming receptor.

In some embodiments, the DNA template comprises, in a 5′ to 3′ direction: the inducible promoter; the chimeric antigen receptor, the constitutive promoter; and the priming receptor.

In some embodiments, the DNA template comprises, in a 5′ to 3′ direction: the constitutive promoter; the priming receptor the inducible promoter; and the chimeric antigen receptor.

In some embodiments, the DNA template further comprises a self-excising 2A peptide (P2A).

In some embodiments, the P2A is at the 3′ end of the DNA template.

In some embodiments, the DNA template further comprises a woodchuck hepatitis virus post-translational regulatory element (WPRE).

In some embodiments, the priming receptor comprises, in an N terminus to C terminus direction: an extracellular antigen-binding domain having a binding affinity for an antigen; a transmembrane domain comprising one or more ligand-inducible proteolytic cleavage sites; and an intracellular domain comprising a human or humanized transcriptional effector, wherein binding of an antigen to the extracellular antigen-binding domain results in cleavage at the ligand-inducible proteolytic cleavage site thereby releasing the intracellular domain.

In some embodiments, the priming receptor further comprises a juxtamembrane domain (JMD) positioned between the transmembrane domain and the intracellular domain.

In some embodiments, the transcription factor binds to the inducible promoter and induces expression of the CAR.

In some embodiments, the CAR comprises, from N-terminus to C-terminus, an extracellular antigen-binding domain having a binding affinity for an antigen; a transmembrane domain; an intracellular co-stimulatory domain; and an intracellular activation domain.

In some embodiments, the priming receptor and the CAR bind different antigens.

In some embodiments, the priming receptor and the CAR bind the same antigen.

In another aspect, provided herein are methods of editing a primary immune cell, comprising: providing a ribonucleoprotein complex (RNP)-DNA template complex, wherein the RNP comprises a nuclease domain and a guide RNA, wherein the size of the DNA template is greater than or equal to 5 kilobase nucleotides in size, and wherein the 5′ and 3′ ends of the DNA template comprise nucleotide sequences that are homologous to genomic sequences flanking an insertion site in the genome of the primary immune cell; non-virally introducing the RNP-DNA template complex into the primary immune cell, wherein the guide RNA specifically hybridizes to a target region of the genome of the primary immune cell, and wherein the nuclease domain cleaves the target region to create the insertion site in the genome of the primary immune cell; and editing the primary immune cell via insertion of the DNA template into the insertion site in the genome of the primary immune cell.

In some embodiments, non-virally introducing comprises electroporation.

In some embodiments, the nuclease domain comprises a CRISPR-associated endonuclease (Cas), optionally a Cas9 nuclease.

In some embodiments, the size of the DNA template is greater than or equal to about 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb. 9.9 kb, 10.0 kb or any size of DNA template in between these sizes.

In some embodiments, the size of the DNA template is about 5 kb to about 10 kb, about 5 kb to about 9 kb, about 5 kb to about 8 kb, about 5 kb to about 7 kb, about 5 kb to about 6 kb, about kb 6 to about 10 kb, about 6 kb to about 9 kb, about 6 kb to about 8 kb, about 6 kb to about 7 kb, about 7 kb to about 10 kb, about 7 kb to about 9 kb, about 7 kb to about 8 kb, about 8 kb to about 10 kb, about 8 kb to about 9 kb, or about 9 kb to about 10 kb.

In some embodiments, the target region of the genome of the cell is a T Cell Receptor Alpha Constant (TRAC) locus or a genomic safe harbor (GSH).

In some embodiments, the DNA template is a double-stranded DNA template or a single-stranded DNA template.

In some embodiments, the DNA template is a linear DNA template or a circular DNA template, optionally wherein the circular DNA template is a plasmid.

In some embodiments, the DNA template comprises a heterologous sequence.

In some embodiments, the DNA template comprises a gene.

In some embodiments, the DNA template comprises a priming receptor comprising a transcription factor.

In some embodiments, the DNA template comprises a chimeric antigen receptor (CAR).

In some embodiments, the DNA template comprises a chimeric antigen receptor (CAR) and a priming receptor comprising a transcription factor.

In some embodiments, the DNA template comprises an inducible promoter operably linked to the chimeric antigen receptor.

In some embodiments, the DNA template further comprises a constitutive promoter operably linked to the priming receptor.

In some embodiments, the DNA template further comprises an inducible promoter operably linked to the chimeric antigen receptor and a constitutive promoter operably linked to the priming receptor.

In some embodiments, the DNA template comprises, in a 5′ to 3′ direction: the inducible promoter; the chimeric antigen receptor, the constitutive promoter; and the priming receptor.

In some embodiments, the DNA template comprises, in a 5′ to 3′ direction: the constitutive promoter; the priming receptor, the inducible promoter; and the chimeric antigen receptor.

In some embodiments, the DNA template further comprises a self-excising 2A peptide (P2A).

In some embodiments, the P2A nucleic acid is at the 3′ end of the DNA template.

In some embodiments, the DNA template further comprises a woodchuck hepatitis virus post-translational regulatory element (WPRE).

In some embodiments, the WPRE is at the 3′ end of the nucleic acid encoding the CAR and at the 5′ end of the nucleic acid encoding the priming receptor or wherein the WPRE is at the 3′ end of the nucleic acid encoding the priming receptor and at the 5′ end of the nucleic acid encoding the CAR.

In some embodiments, the priming receptor comprises, in an N terminus to C terminus direction: an extracellular antigen-binding domain having a binding affinity for an antigen; a transmembrane domain comprising one or more ligand-inducible proteolytic cleavage sites; and an intracellular domain comprising a human or humanized transcriptional effector, wherein binding of an antigen to the extracellular antigen-binding domain results in cleavage at the ligand-inducible proteolytic cleavage site thereby releasing the intracellular domain.

In some embodiments, the priming receptor further comprises a juxtamembrane domain (JMD) positioned between the transmembrane domain and the intracellular domain.

In some embodiments, the transcription factor binds to the inducible promoter and induces expression of the CAR.

In some embodiments, the CAR comprises, from N-terminus to C-terminus, an extracellular antigen-binding domain having a binding affinity for an antigen; a transmembrane domain; an intracellular co-stimulatory domain; and an intracellular activation domain.

In some embodiments, the priming receptor and the CAR bind different antigens.

In some embodiments, the priming receptor and the CAR bind the same antigen.

In some embodiments, the immune cell is a primary human immune cell.

In some embodiments, the primary immune cell is an autologous immune cell.

In some embodiments, the primary immune cell is a natural killer (NK) cell, a T cell, a CD8+ T cell, a CD4+ T cell, a primary T cell, or a T cell progenitor.

In some embodiments, the primary immune cell is a primary T cell.

In some embodiments, the primary immune cell is a primary human T cell.

In some embodiments, the primary immune cell is virus-free.

In some embodiments, the method further comprises obtaining the immune cell from a patient and introducing the plasmid in vitro.

In another aspect, provided herein are methods of editing a primary immune cell, comprising: providing a ribonucleoprotein complex (RNP)-DNA template complex, wherein the RNP comprises a nuclease domain and a guide RNA, wherein the size of the DNA template is greater than or equal to 5 kilobase nucleotides in size, wherein the DNA template comprises a chimeric antigen receptor (CAR) and a priming receptor comprising a transcription factor and wherein the 5′ and 3′ ends of the DNA template comprise nucleotide sequences that are homologous to genomic sequences flanking an insertion site in the genome of the primary immune cell; non-virally introducing the RNP-DNA template complex into the primary immune cell, wherein the guide RNA specifically hybridizes to a target region of the genome of the primary immune cell, and wherein the nuclease domain cleaves the target region to create the insertion site in the genome of the primary immune cell; and editing the primary immune cell via insertion of the DNA template into the insertion site in the genome of the primary immune cell.

### BRIEF DESCRIPTION OF THE SEVERAL VIEWS OF THE DRAWINGS

These and other features, aspects, and advantages of the present disclosure will become better understood with regard to the following description, and accompanying drawings, where:

FIG. 1 shows an exemplary plasmid of the invention.

FIG. 2 shows an exemplary method of producing engineered immune cells.

FIG. 3 shows a schematic of an experimental protocol.

FIG. 4 provides experimental results showing desired receptor expression and control.

FIG. 5 shows a schematic of an experimental protocol.

FIG. 6 provides experimental results showing low amounts of unwanted CAR expression.

FIG. 7 shows a schematic showing the GS94 integration site on Chromosome 11.

FIG. 8A includes plots showing CAR induction and primeR expression of engineered T cells after 48 hours of coculturing with K562-PrimeR cells. FIG. 8B includes plots showing the cytotoxity and cytokine secretion levels for engineered T cells 48 hours of coculturing with K562-primeR/CAR cells.

FIG. 9A includes a schematic showing the experimental overview for evaluating the effect of integration site on cytotoxicity. FIG. 9B includes plots showing the measured cytotoxicity for engineered T cells cocultured for 48 hours with the K562-primeR/CAR or K562-CAR cells.

FIG. 10A includes a schematic showing the experimental overview for evaluating the effect of integration site on cytokine secretion. FIG. 10B includes plots showing the measured cytokine levels for engineered T cells cocultured for 48 hours with K562-primeR/CAR cells.

FIG. 11 includes a schematic showing the in vitro experiment conducted to determine the effect of integration site on primeR-independent CAR expression. “Flow” refers to flow cytometry and “restim” refers to repetitive CD3/CD28 stimulation of the engineered T cells. “EP” refers to electroporation.

FIGS. 12A and 12B include plots showing the stability of PrimeR expression over time when using the indicated integration sites. “Flow” refers to flow cytometry and “restim” refers to repetitive CD3/CD28 stimulation of the engineered T cells. “EP” refers to electroporation. In FIG. 12B, the PrimeR expression is normalized to the expression from using the TRAC integration site.

FIG. 13A includes a schematic showing the iGuide-Seq assay technique. FIG. 13B includes a plot showing the on-target efficiency, using iGuide Seq assay, for the indicated integration sites. FIG. 13C includes schematics from the iGuide-Seq analysis showing that GS94 had no reproducible putatitve off-targets across two donors. Figure discloses SEQ ID NOS 127-137, 127, 128, and 138-146, respectively, in order of appearance.

FIG. 14 includes a schematic showing the iGuide-Seq workflow and data.

FIG. 15 includes a plot showing rhAmp-seq analysis of putative off-target sites identified by iGUIDE-seq and Elevation prediction.

FIG. 16 includes plots showing RNA-seq analysis of cells with GS94, GS102 and TRAC knock-in of primeR/CAR circuits. Scatterplot of gene expression in cells with integration at the GS94 locus (y-axis) vs cells with integration at either the TRAC or the GS102 locus (x-axis) in two donors. The light gray dots correspond to ETS1 and FLI1. In dark gray are the genes that were found to be differentially expressed using edgeR (fold-change >0, FDR-corrected p-value <0.01, average counts-per-million across compared conditions at least 2).

FIG. 17 includes plots showing the absence of cytokine-independent growth in cells with a primeR/CAR circuit knock-in at GS94.

FIG. 18 shows the expression of a 8.3 kb transgene circuit comprising a primeR and CAR in K562 cells.

FIG. 19A shows a diagram of a 4.6 kb cassette that was inserted into the GS94 safe harbor locus. FIG. 19B shows a diagram of a 8.3 kb cassette that was inserted into the GS94 safe harbor locus.

### DETAILED DESCRIPTION

The present invention provides novel engineered immune cells and methods for making and using such cells. The engineered immune cells include a transgene inserted into the immune cells' genome, which encodes genes for an engineered priming receptor and a chimeric antigen receptor (CAR). Initially, the engineered immune cells express the priming receptor encoded by the plasmid on the cells' surface, but do not express the CAR. The priming receptors include a cleavable transcription factor. When an immune cell of the invention encounters a target cell, the priming receptor binds to a cognate ligand on the target cell. This causes the priming receptor to release the cleavable transcription factor. Release of the transcription factor causes the engineered immune cell to express the CAR encoded by the integrated transgene on the surface of the cell.

When expressed on the surface of the immune cell, the CAR is available to bind to a cognate ligand on a target cell. Binding of the CAR to its target ligand triggers a cytotoxic response by the engineered immune cell, which kills the target cell.

The engineered immune cells of the present invention have several advantages over prior engineered immune cells. The embodiment described below demonstrates that a synthetic circuit can be delivered into the T cell genome using targeted non-viral delivery, and that circuit fidelity can be maintained after genomic integration. This enhances the safety profile and broadens the therapeutic window of the cells, as targeted integration into the TRAC promoter reduces the risk of off-target gene insertion into the genome, as occurs via lentivirus gene delivery. In addition, the circuit provides additional safety features. For example, the cytotoxic CAR is not available for activation, because it is not expressed, until it encounters a target cell with the priming receptor's cognate ligand. Moreover, because the CAR is not initially expressed, the cells are less likely to exhibit exhaustion, differentiation, tonic signaling, and activation-induced immune cell death, while concurrently exhibiting high proliferation and persistence potential.

### Definitions

Terms used in the claims and specification are defined as set forth below unless otherwise specified.

As used herein, the term “gene” refers to the basic unit of heredity, consisting of a segment of DNA arranged along a chromosome, which codes for a specific protein or segment of protein. A gene typically includes a promoter, a 5′ untranslated region, one or more coding sequences (exons), optionally introns, and a 3′ untranslated region. The gene may further comprise a terminator, enhancers and/or silencers.

As used herein, the term “locus” refers to a specific, fixed physical location on a chromosome where a gene or genetic marker is located.

The term “safe harbor locus” refers to a locus at which genes or genetic elements can be incorporated without disruption to expression or regulation of adjacent genes. These safe harbor loci are also referred to as safe harbor sites (SHS). As used herein, a safe harbor locus refers to an “integration site” or “knock-in site” at which a sequence encoding a transgene, as defined herein, can be inserted. In some embodiments the insertion occurs with replacement of a sequence that is located at the integration site. In some embodiments, the insertion occurs without replacement of a sequence at the integration site. Examples of integration sites contemplated are provided in Table 1.

As used herein, the term “insert” refers to a nucleotide sequence that is integrated (inserted) at a target locus or safe harbor site. The insert can be used to refer to the genes or genetic elements that are incorporated at the target locus or safe harbor site using, for example, homology-directed repair (HDR) CRISPR/Cas9 genome-editing or other methods for inserting nucleotide sequences into a genomic region known to those of ordinary skill in the art.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, Cas9 in complex with guide and activating RNA to recognize and cleave foreign nucleic acid. Guide RNAs having the activity of both a guide RNA and an activating RNA are also known in the art. In some cases, such dual activity guide RNAs are referred to as a small guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. The Cas9 nuclease domain can be optimized for efficient activity or enhanced stability in the host cell.

As used herein, the term “Cas9” refers to an RNA-mediated nuclease (e.g., of bacterial or archeal orgin, or derived therefrom). Exemplary RNA-mediated nuclases include the foregoing Cas9 proteins and homologs thereof, and include but are not limited to, CPF1 (See, e.g., Zetsche et al., Cell, Volume 163, Issue 3, p759-771, 22 Oct. 2015). Similarly, as used herein, the term “Cas9 ribonucleoprotein” complex and the like refers to a complex between the Cas9 protein, and a crRNA (e.g., guide RNA or small guide RNA), the Cas9 protein and a trans-activating crRNA (tracrRNA), the Cas9 protein and a small guide RNA, or a combination thereof (e.g., a complex containing the Cas9 protein, a tracrRNA, and a crRNA guide RNA).

As used herein, the terms “T lymphocyte” and “T cell” are used interchangeably and refer to cells that have completed maturation in the thymus, and identify certain foreign antigens in the body. The terms also refer to the major leukocyte types that have various roles in the immune system, including activation and deactivation of other immune cells. The T cell can be any T cell such as a cultured T cell, e.g., a primary T cell, or a T cell derived from a cultured T cell line, e.g., a Jurkat, SupT1, etc., or a T cell obtained from a mammal. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. The T cell can be a CD3+ cell. T cells can be CD4⁺, CD8⁺, or CD4⁺ and CD8⁺. The T cell can be any type of T cell, CD4+/CD8+ double positive T cells, CD4+ helper T cells (e.g. Th1 and Th2 cells), CD8+ T cells (e.g. cytotoxic T cells), peripheral Including but not limited to blood mononuclear cells (PBMC), peripheral blood leukocytes (PBL), tumor infiltrating lymphocytes (TIL), memory T cells, naive T cells, regulatory T cells, γδ T cells, etc. It can be any T cell at any stage of development. Additional types of helper T cells include Th3 (Treg) cells, Th17 cells, Th9 cells, or Tfh cells. Additional types of memory T cells include cells such as central memory T cells (Tcm cells), effector memory T cells (Tem cells and TEMRA cells). A T cell can also refer to a genetically modified T cell, such as a T cell that has been modified to express a T cell receptor (TCR) or a chimeric antigen receptor (CAR). T cells can also be differentiated from stem cells or progenitor cells.

“CD4+ T cells” refers to a subset of T cells that express CD4 on their surface and are associated with a cellular immune response. CD4+ T cells are characterized by a post-stimulation secretion profile that can include secretion of cytokines such as IFN-γ, TNF-α, IL-2, IL-4 and IL-10. “CD4” is a 55 kD glycoprotein originally defined as a differentiation antigen on T lymphocytes, but was also found on other cells including monocytes/macrophages. The CD4 antigen is a member of the immunoglobulin superfamily and has been implicated as an associative recognition element in MHC (major histocompatibility complex) class II restricted immune responses. On T lymphocytes, the CD4 antigen defines a helper/inducer subset.

“CD8+ T cells” refers to a subset of T cells that express CD8 on their surface, are MHC class I restricted, and function as cytotoxic T cells. The “CD8” molecule is a differentiation antigen present on thymocytes, as well as on cytotoxic and suppressor T lymphocytes. The CD8 antigen is a member of the immunoglobulin superfamily and is an associative recognition element in major histocompatibility complex class I restriction interactions.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit⁺ and lin⁻. In some cases, human hematopoietic stem cells are identified as CD34⁺, CD59⁺, Thy1/CD90⁺, CD38^{lo/-}, C-kit/CD117⁺, lin⁻. In some cases, human hematopoietic stem cells are identified as CD34⁻, CD59⁺, Thy1/CD90⁺, CD38^{lo/-}, C-kit/CD117⁺, lin⁻. In some cases, human hematopoietic stem cells are identified as CD133⁺, CD59⁺, Thy1/CD90⁺, CD38^{lo/-}, C-kit/CD117⁺, lin⁻. In some cases, mouse hematopoietic stem cells are identified as CD34^{lo/-}, SCA-1⁺, Thy1^{+/lo}, CD38⁺, C-kit⁺, lin⁻. In some cases, the hematopoietic stem cells are CD150⁺CD48⁻CD244⁻.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes.

As used herein, the phrase “immune cell” is inclusive of all cell types that give rise to immune cells, including hematopoietic cells, pluripotent stem cells, and induced pluripotent stem cells (iPSCs). In some embodiments, the immune cell is a B cell, macrophage, a natural killer (NK) cell, an induced pluripotent stem cell (iPSC), a human pluripotent stem cell (HSPC), a T cell or a T cell progenitor or dendritic cell. In some embodiments, the cell is an innate immune cell.

As used herein, the term “primary” in the context of a primary cell or primary stem cell refers to a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized, e.g., directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

As used herein, the term “ex vivo” generally includes experiments or measurements made in or on living tissue, preferably in an artificial environment outside the organism, preferably with minimal differences from natural conditions.

As used herein, the term “construct” refers to a complex of molecules, including macromolecules or polynucleotides.

As used herein, the term “integration” refers to the process of stably inserting one or more nucleotides of a construct into the cell genome, i.e., covalently linking to a nucleic acid sequence in the chromosomal DNA of the cell. It may also refer to nucleotide deletions at a site of integration. Where there is a deletion at the insertion site, “integration” may further include substitution of the endogenous sequence or nucleotide deleted with one or more inserted nucleotides.

As used herein, the term “exogenous” refers to a molecule or activity that has been introduced into a host cell and is not native to that cell. The molecule can be introduced, for example, by introduction of the encoding nucleic acid into host genetic material, such as by integration into a host chromosome, or as non-chromosomal genetic material, such as a plasmid. Thus, the term, when used in connection with expression of an encoding nucleic acid, refers to the introduction of the encoding nucleic acid into a cell in an expressible form. The term “endogenous” refers to a molecule or activity that is present in a host cell under natural, unedited conditions. Similarly, the term, when used in connection with expression of the encoding nucleic acid, refers to expression of the encoding nucleic acid that is contained within the cell and not introduced exogenously.

The term “heterologous” refers to a nucleic acid or polypeptide sequence or domain which is not native to a flanking sequence, e.g., wherein the heterologous sequence is not found in nature coupled to the nucleic acid or polypeptide sequences occurring at one or both ends.

The term “homologous” refers to a nucleic acid or polypeptide sequence or domain which is native to a flanking sequence, e.g., wherein the homologous sequence is found in nature coupled to the nucleic acid or polypeptide sequences occurring at one or both ends.

As used herein, a “polynucleotide donor construct” refers to a nucleotide sequence (e.g. DNA sequence) that is genetically inserted into a polynucleotide and is exogenous to that polynucleotide. The polynucleotide donor construct is transcribed into RNA and optionally translated into a polypeptide. The polynucleotide donor construct can include prokaryotic sequences, cDNA from eukaryotic mRNA, genomic DNA sequences from eukaryotic (e.g., mammalian) DNA, and synthetic DNA sequences. For example, the polynucleotide donor construct can be a miRNA, shRNA, natural polypeptide (i.e., a naturally occurring polypeptide) or fragment thereof or a variant polypeptide (e.g. a natural polypeptide having less than 100% sequence identity with the natural polypeptide) or fragments thereof.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequence that are perfectly complementary or substantially complementary (e.g., having 1-4 mismatches) to a genomic sequence in a cell.

As used herein, the term “transgene” refers to a polynucleotide that has been transferred naturally, or by any of a number of genetic engineering techniques from one organism to another. It is optionally translated into a polypeptide. It is optionally translated into a recombinant protein. A “recombinant protein” is a protein encoded by a gene—recombinant DNA— that has been cloned in a system that supports expression of the gene and translation of messenger RNA (see expression system). The recombinant protein can be a therapeutic agent, e.g. a protein that treats a disease or disorder disclosed herein. As used, transgene can refer to a polynucleotide that encodes a polypeptide. A transgene can also refer to a non-encoding sequence, such as, but not limited to shRNAs, miRNAs, and miRs.

The terms “protein,” “polypeptide,” and “peptide” are used herein interchangeably.

As used herein, the term “operably linked” or “operatively linked” refers to the binding of a nucleic acid sequence to a single nucleic acid fragment such that one function is affected by the other. For example, if a promoter is capable of affecting the expression of a coding sequence or functional RNA (i.e., the coding sequence or functional RNA is under transcriptional control by the promoter), the promoter is operably linked thereto. Coding sequences can be operably linked to control sequences in both sense and antisense orientation.

As used herein, the term “developmental cell states” refers to, for example, states when the cell is inactive, actively expressing, differentiating, senescent, etc. developmental cell state may also refer to a cell in a precursor state (e.g., a T cell precursor).

As used, the term “encoding” refers to a sequence of nucleic acids which codes for a protein or polypeptide of interest. The nucleic acid sequence may be either a molecule of DNA or RNA. In preferred embodiments, the molecule is a DNA molecule. In other preferred embodiments, the molecule is a RNA molecule. When present as a RNA molecule, it will comprise sequences which direct the ribosomes of the host cell to start translation (e.g., a start codon, ATG) and direct the ribosomes to end translation (e.g., a stop codon). Between the start codon and stop codon is an open reading frame (ORF). Such terms are known to one of ordinary skill in the art.

The term “inserting” refers to a manipulation of a nucleotide sequence to introduce a non-native sequence. This is done, for example, via the use of restriction enzymes and ligases whereby the DNA sequence of interest, usually encoding the gene of interest, can be incorporated into another nucleic acid molecule by digesting both molecules with appropriate restriction enzymes in order to create compatible overlaps and then using a ligase to join the molecules together. One skilled in the art is very familiar with such manipulations and examples may be found in Sambrook et al. (Sambrook, Fritsch, & Maniatis, “Molecular Cloning: A Laboratory Manual”, 2nd ed., Cold Spring Harbor Laboratory, 1989), which is hereby incorporated by reference in its entirety including any drawings, figures and tables.

As used herein, the term “subject” refers to a mammalian subject. Exemplary subjects include humans, monkeys, dogs, cats, mice, rats, cows, horses, camels, goats, rabbits, pigs and sheep. In certain embodiments, the subject is a human. In some embodiments the subject has a disease or condition that can be treated with an engineered cell provided herein or population thereof. In some aspects, the disease or condition is a cancer.

As used herein, the term “promoter” refers to a nucleotide sequence (e.g. DNA sequence) capable of controlling the expression of a coding sequence or functional RNA. The promoter sequence consists of proximal and more distal upstream elements, the latter elements often referred to as enhancers. A promoter can be derived from natural genes in its entirety, can be composed of different elements from different promoters found in nature, and/or may comprise synthetic DNA segments. A promoter, as contemplated herein, can be endogenous to the cell of interest or exogenous to the cell of interest. It is appreciated by those skilled in the art that different promoters can induce gene expression in different tissue or cell types, or at different developmental stages, or in response to different environmental conditions. As is known in the art, a promoter can be selected according to the strength of the promoter and/or the conditions under which the promoter is active, e.g., constitutive promoter, strong promoter, weak promoter, inducible/repressible promoter, tissue specific Or developmentally regulated promoters, cell cycle-dependent promoters, and the like.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some embodiments, the promoter can be a spatially restricted and/or temporally restricted promoter (e.g., a tissue specific promoter, a cell type specific promoter, etc.). See for example US Publication 20180127786, the disclosure of which is herein incorporated by reference in its entirety.

Gene editing, as contemplated herein, may involve a gene (or nucleotide sequence) knock-in or knock-out. As used herein, the term “knock-in” refers to an addition of a DNA sequence, or fragment thereof into a genome. Such DNA sequences to be knocked-in may include an entire gene or genes, may include regulatory sequences associated with a gene or any portion or fragment of the foregoing. For example, a polynucleotide donor construct encoding a recombinant protein may be inserted into the genome of a cell carrying a mutant gene. In some embodiments, a knock-in strategy involves substitution of an existing sequence with the provided sequence, e.g., substitution of a mutant allele with a wild-type copy. On the other hand, the term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant (e.g., non-coding) sequence.

As used herein, the term “non-homologous end joining” or NHEJ refers to a cellular process in which cut or nicked ends of a DNA strand are directly ligated without the need for a homologous template nucleic acid. NHEJ can lead to the addition, the deletion, substitution, or a combination thereof, of one or more nucleotides at the repair site.

As used herein, the term “homology directed repair” or HDR refers to a cellular process in which cut or nicked ends of a DNA strand are repaired by polymerization from a homologous template nucleic acid. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

As used herein, a single-stranded DNA template or a double-stranded DNA template refers to a DNA oligonucleotide that can be used by a cell as a template for HDR. Generally, the single-stranded DNA template or a double-stranded DNA template has at least one region of homology to a target site. In some cases, the single-stranded DNA template or double-stranded DNA template has two homologous regions flanking a region that contains a heterologous sequence to be inserted at a target cut site.

The terms “vector” and “plasmid” are used interchangeably and as used herein refer to polynucleotide vehicles useful to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, cosmids, and artificial chromosomes.

As used herein, the phrase “introducing” in the context of introducing a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used herein, the phrase “subject in need thereof” refers to a subject that exhibits and/or is diagnosed with one or more symptoms or signs of a disease or disorder as described herein.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “composition” refers to a mixture that contains, e.g., an engineered cell or protein contemplated herein. In some embodiments, the composition may contain additional components, such as adjuvants, stabilizers, excipients, and the like. The term “composition” or “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The term “ameliorating” refers to any therapeutically beneficial result in the treatment of a disease state, e.g., a cancer disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

As used herein, the term “effective amount” refers to the amount of a compound (e.g., a compositions described herein, cells described herein) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

It must be noted that, as used in the specification and the appended claims, the singular forms “a,” “an” and “the” include plural referents unless the context clearly dictates otherwise.

Recombinant Nucleic Acids and Vectors

In some embodiments, the present disclosure contemplates recombinant nucleic acid inserts that comprise one or more transgenes. The transgene can encode a therapeutic protein, an antibody, a peptide, a suicide gene, an apoptosis gene or any other gene of interest. In some embodiments, the transgene encodes a priming receptor. In some embodiments, the transgene encodes a chimeric antigen receptor. In some embodiments, the insert comprises a priming receptor transgene and a chimeric antigen receptor transgene.

The insert can also comprise a self-cleaving peptide. Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Chang et al. “Cleavage efficient 2A peptides for high level monoclonal antibody expression in CHO cells,” MAbs 7(2): 403-412 (2015)).

The insert can also comprise a WPRE element. WPRE elements are generally described in Higashimoto, T., et al. Gene Ther 14, 1298-1304 (2007); and Zufferey, R., et al. J Virol. 1999 April; 73(4):2886-92., both of which are hereby incorporated by reference.

Priming Receptors

In certain aspects of the present disclosure, the priming receptor is a synthetic circuit receptor based on the Notch protein. Binding of a natural Notch receptor to a cognate ligand, such as those from the Delta family of proteins, causes intramembrane proteolysis that cleaves an intracellular fragment of the Notch protein. This intracellular fragment is a transcriptional regulator that only functions when cleaved from Notch. Cleavage may occur by sequential proteolysis by ADAM metalloprotease and the gamma-secretase complex. This intracellular fragment enters the nucleus of a cell and activates cell-cell signaling genes. In contrast to a natural Notch protein, a synNotch priming receptor replaces the natural Notch intracellular fragment with one that causes the gene encoding the CAR to activate upon release from the priming receptor.

Notch receptors have a modular domain organization. The ectodomains of Notch receptors consist of a series of N-terminal epidermal growth factor (EGF)-like repeats that are responsible for ligand binding. In synthetic Notch receptors or priming receptors, the Notch ligand-binding domain is replaced with a ligand binding domain that binds a selected target ligand or antigen. The EGF repeats are followed by three LIN-12/Notch repeat (LNR) modules, which are unique to Notch receptors, and are widely reported to participate in preventing premature receptor activation. The heterodimerization (HD) domain of Notch1 is divided by furin cleavage, so that its N-terminal part terminates the extracellular subunit, and its C-terminal half constitutes the beginning of the transmembrane subunit. Following the extracellular region, the receptor has a transmembrane segment and an intracellular domain (ICD), which includes a transcriptional regulator.

Multiple forms of priming receptors can be used in the methods, cells, and nucleic acids as described herein. One type of priming receptor contemplated for use in the methods and cells herein comprise a heterologous extracellular ligand binding domain, a linking polypeptide having substantial sequence identity with a Notch receptor including the NRR, a TMD, and an ICD. “Fn Notch” receptors comprise a heterologous extracellular ligand binding domain, a linking polypeptide having substantial sequence identity with a Robo receptor (such as a mammalian Robo1, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor (lacking the NRR), a TMD, and an ICD. “Minimal Tinker Notch” receptors comprise a heterologous extracellular ligand binding domain, a linking polypeptide lacking substantial sequence identity with a Notch receptor (e.g., a synthetic (GGS)n polypeptide sequence), a TMD, and an ICD. “Hinge Notch” receptors comprise a heterologous extracellular ligand binding domain, a hinge sequence comprising an oligomerization domain (i.e., a domain that promotes dimerization, trimerization, or higher order multimerization with a synthetic receptor and/or an existing host receptor), a TMD, and an ICD. All of these receptor classes are synthetic, recombinant, and do not occur in nature. In some embodiments, the non-naturally occurring receptors disclosed herein bind a target cell-surface displayed ligand, which triggers proteolytic cleavage of the receptors and release of a transcriptional regulator that modulates a custom transcriptional program in the cell. In some embodiments, the priming receptor does not include a LIN-12-Notch repeat (LNR) and/or a heterodimerization domain (HD) of a Notch receptor.

Priming Receptor Extracellular Domain

The priming receptor comprises an extracellular domain. In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment thereof. In some embodiments, the antigen-binding moiety is selected from the group consisting of an antibody, a nanobody, a diabody, a triabody, or a minibody, a F(ab′)2 fragment, a Fab fragment, a single chain variable fragment (scFv), and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity. An antibody that “selectively binds” an antigen is an antigen-binding moiety that binds the antigen with high affinity and does not significantly bind other unrelated antigens. In some embodiments, the extracellular antigen-binding domain binds to Alkaline phosphatase, germ cell type (ALPG). Additional antigens are described in WO201061872.

Transmembrane Domain

As described above, the chimeric polypeptides of the disclosure include a TMD comprising one or more ligand-inducible proteolytic cleavage sites.

Generally, the TMD suitable for the chimeric receptors disclosed herein can be any transmembrane domain of a Type 1 transmembrane receptor including at least one gamma-secretase cleavage site. Detailed description of the structure and function of the gamma-secretase complex as well as its substrate proteins, including amyloid precursor protein (APP) and Notch, can, for example, be found in a recent review by Zhang et al, Frontiers Cell Neurosci (2014). Non limiting suitable TMDs from Type 1 transmembrane receptors include those from CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one gamma secretase cleavage site. Additional TMDs suitable for the compositions and methods described herein include, but are not limited to, transmembrane domains from Type 1 transmembrane receptors IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, YASN, FLT1, CDH5, PKHD1, NECTIN1, PCDHGC3, NRG1, LRP1B, CDH2, NRG2, PTPRK, SCN2B, Nradd, and PTPRM. In some embodiments, the TMD of the chimeric polypeptides or Notch receptors of the disclosure is a TMD derived from the TMD of a member of the calsyntenin family, such as, alcadein alpha and alcadein gamma. In some embodiments, the TMD of the chimeric polypeptides or Notch receptors of the disclosure is a TMD known for Notch receptors. In some embodiments, the TMD of the chimeric polypeptides or Notch receptors of the disclosure is a TMD derived from a different Notch receptor. For example, in a Mini Notch based on human Notch1, the Notch1 TMD can be substituted with a Notch2 TMD, Notch3 TMD, Notch4 TMD, or a Notch TMD from a non-human animal such as Danio rerio, Drosophila melanogaster, Xenopus laevis, or Gallus gallus.

In some embodiments, the priming receptor comprises a Notch cleavage site, such as S2 or S3. Additional proteolytic cleavage sites suitable for the compositions and methods disclosed herein include, but are not limited to, a metalloproteinase cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Yal-Gly-Arg. Another example of a protease cleavage site that can be included in a proteolytically cleavable linker is a tobacco etch vims (TEV) protease cleavage site, e.g., Glu-Asn-Leu-Tyr-Thr-Gln-Ser (SEQ ID NO: 121), where the protease cleaves between the glutamine and the serine. Another example of a protease cleavage site that can be included in a proteolytically cleavable linker is an enterokinase cleavage site, e.g., Asp-Asp-Asp-Asp-Lys (SEQ ID NO: 122), where cleavage occurs after the lysine residue. Another example of a protease cleavage site that can be included in a proteolytically cleavable linker is a thrombin cleavage site, e.g., Leu-Val-Pro-Arg (SEQ ID NO: 123). Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr vims proteas, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrixmetalloprotemase (MT-MMP), stromelysin 3 (or MMP-11), thermo lysin, fibroblast collagenase and stromelysin-1, matrix metalloproteinase 13 (collagenase-3), tissue-type plasminogen activator (tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the receptor is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

In some embodiments, the amino acid substitution(s) within the TMD includes one or more substitutions within a “GV” motif of the TMD. In some embodiments, at least one of such substitution(s) comprises a substitution to alanine. Additional sequences and substitutions are described in WO2021061872, hereby incorporated by reference in its entirety.

Intracellular Domain

In some embodiments the priming receptor comprises one or more intracellular domains from or derived from a transcriptional regulator. Transcriptional regulators either activate or repress transcription from cognate promoters. Transcriptional activators typically bind nearby to transcriptional promoters and recruit RNA polymerase to directly initiate transcription. Transcriptional repressors bind to transcriptional promoters and sterically hinder transcriptional initiation by RNA polymerase. Other transcriptional regulators serve as either an activator or a repressor depending on where it binds and cellular conditions. Accordingly, as used herein, a “transcriptional activation domain” refers to the domain of a transcription factor that interacts with transcriptional control elements and/or transcriptional regulatory proteins (i.e., transcription factors, RNA polymerases, etc.) to increase and/or activate transcription of one or more genes. Non-limiting examples of transcriptional activation domains include: a herpes simplex virus VP16 activation domain, VP64 (which is a tetrameric derivative of VP16), HIV TAT, a NFkB p65 activation domain, p53 activation domains 1 and 2, a CREB (cAMP response element binding protein) activation domain, an E2A activation domain, NFAT (nuclear factor of activated T-cells) activation domain, yeast Gal4, yeast GCN4, yeast HAP1, MLL, RTG3, GLN3, OAF1, PIP2, PDR1, PDR3, PHO4, LEU3 glucocorticoid receptor transcription activation domain, B-cell POU homeodomain protein Oct2, plant Ap2, or any others known to one or ordinary skill in the art. In some embodiments, the transcriptional regulator is selected from Gal4-VP16, Gal4-VP64, tetR-VP64, ZFHD1-YP64, Gal4-KRAB, and HAP1-VP16. In some embodiments, the transcriptional regulator is Gal4-VP64. A transcriptional activation domain can comprise a wild-type or naturally occurring sequence, or it can be a modified, mutant, or derivative version of the original transcriptional activation domain that has the desired ability to increase and/or activate transcription of one or more genes. In some embodiments, the transcriptional regulator can further include a nuclear localization signal.

DNA-Binding Domain

In some embodiments of the aspects described herein, a synthetic protein comprises one or more intracellular “DNA-binding domains” (or “DB domains”). Such “DNA-binding domains” refer to sequence-specific DNA binding domains that bind a particular DNA sequence element. Accordingly, as used herein, a “sequence-specific DNA-binding domain” refers to a protein domain portion that has the ability to selectively bind DNA having a specific, predetermined sequence. A sequence-specific DNA binding domain can comprise a wild-type or naturally occurring sequence, or it can be a modified, mutant, or derivative version of the original domain that has the desired ability to bind to a desired sequence. In some embodiments, the sequence-specific DNA binding domain is engineered to bind a desired sequence. Non-limiting examples of proteins having sequence-specific DNA binding domains that can be used in synthetic proteins described herein include HNF1a, Gal4, GCN4, reverse tetracycline receptor, THY1, SYN1, NSE/RU5′, AGRP, CALB2, CAMK2A, CCK, CHAT, DLX6A, EMX1, zinc finger proteins or domains thereof, CRISPR/Cas proteins, such as Cas9, Cas3, Cas4, Cas5, Cas5e (or CasD), Cash, Cas6e, Cas6f, Cas7, Cas8a1, Cas8a2, Cas8b, Cas8c, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cul96, and TALES.

In those embodiments where a CRISPR/Cas-like protein is used, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the functions of the systems described herein. For example, a CRISPR enzyme that is used as a DNA binding protein or domain thereof can be mutated with respect to a corresponding wild-type enzyme such that the mutated CRISPR or domain thereof lacks the ability to cleave a nucleic acid sequence containing a DNA binding domain target site. For example, a D10A mutation can be combined with one or more of H840A, N854A, or N863A mutations to produce a Cas9 enzyme substantially lacking all DNA cleavage activity.

Juxtamembrane Domain

The ECD and the TMD, or the TMD and the ICD, can be linked to each other with a linking polypeptide, such as a juxtamembrane domain. “SynNotch” receptors comprise a heterologous extracellular ligand-binding domain, a linking polypeptide having substantial sequence identity with a Notch receptor JMD (including the NRR), a TMD, and an ICD. “Fn Notch” receptors comprise a heterologous extracellular ligand binding domain, a linking polypeptide having substantial sequence identity with a Robo receptor (such as a mammalian Robo1, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor JMD but lacking the NRR (the LIN-12-Notch repeat (LNR) modules, and the heterodimerization domain), a TMD, and an ICD. “Minimal Linker Notch” receptors comprise a heterologous extracellular ligand-binding domain, a linking polypeptide lacking substantial sequence identity with a Notch receptor (for example, without limitation, having a synthetic (GGS)n polypeptide sequence), a TMD, and an ICD. “Hinge Notch” receptors comprise a heterologous extracellular ligand-binding domain, a hinge sequence comprising an oligomerization domain (i.e., a domain that promotes dimerization, trimerization, or higher order multimerization with a synthetic receptor and/or an existing host receptor), a TMD, and an ICD.

In some embodiments, the priming receptor comprises a juxtamembrane domain (JMD) peptide in between the extracellular domain and the transmembrane domain. In some embodiments, the priming receptor comprises a juxtamembrane domain (JMD) peptide in between the transmembrane domain and the intracellular domain. In some embodiments, the JMD peptide comprises an LWF motif. The use of LWF motifs in receptor constructs is described in U.S. Pat. No. 10,858,443, hereby incorporated by reference in its entirety. In some embodiments, the JMD peptide has substantial sequence identity to the JMD of Notch1, Notch2, Notch3, and/or Notch4. In some embodiments, the JMD peptide has substantial sequence identity to the Notch1, Notch2, Notch3, and/or Notch4 JMD, but does not include a LIN-12-Notch repeat (LNR) and/or a heterodimerization domain (HD) of a Notch receptor. In some embodiments, the JMD peptide does not have substantial sequence identity to the Notch1, Notch2, Notch3, and/or Notch4 JMD. In some embodiments, the JMD peptide includes an oligimerization domain which promotes formation of dimers, trimers, or higher order assemblages of the receptor. Such JMD peptides are described in WO2021061872, hereby incorporated by reference in its entirety.

In the Mini Notch receptor, the linking polypeptide is derived from a Notch JMD sequence after deletion of the NRR and HD domain. The Notch JMD sequence may be the sequence from Notch1, Notch2, Notch3, or Notch4, and can be derived from a non-human homolog, such as those from Drosophila, Gallus, Danio, and the like. Four to 50 amino acid residues of the remaining Notch sequence can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide, such as the signal transduction level when ligand induced or in the absence of ligand.

In the Minimal Linker Notch receptor, the linking polypeptide does not have substantial sequence identity to a Notch JMD sequence, including the Notch JMD sequence from Notch1, Notch2, Notch3, or Notch4, or a non-human homolog thereof. Four to 50 amino acid residues can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide of the disclosure. The Minimal Linker sequence can be designed to include or omit a protease cleavage site, and can include or omit a glycosylation site or sites for other types of post-translational modification. In some embodiments, the Minimal Linker linker does not comprise a protease cleavage site or a glysosylation site.

In some embodiments, the priming receptor further comprises a hinge. Hinge linkers that can be used in the priming receptor can include an oligomerization domain (e.g., a hinge domain) containing one or more polypeptide motifs that promote oligomer formation of the chimeric polypeptides via intermolecular disulfide bonding. In these instances, within the chimeric receptors disclosed herein, the hinge domain generally includes a flexible polypeptide connector region disposed between the ECD and the TMD. Thus, the hinge domain provides flexibility between the ECD and TMD and also provides sites for intermolecular disulfide bonding between two or more chimeric polypeptide monomers to form an oligomeric complex. In some embodiments, the hinge domain includes motifs that promote dimer formation of the chimeric polypeptides disclosed herein. In some embodiments, the hinge domain includes motifs that promote trimer formation of the chimeric polypeptides disclosed herein (e.g., a hinge domain derived from OX40). Hinge polypeptide sequences suitable for the compositions and methods of the disclosure can be naturally-occurring hinge polypeptide sequences (e.g., those from naturally-occurring immunoglobulins) or can be engineered, designed, or modified so as to provide desired and/or improved properties, e.g., modulating transcription. Suitable hinge polypeptide sequences include, but are not limited to, those derived from IgA, IgD, and IgG subclasses, such as IgG1 hinge domain, IgG2 hinge domain, IgG3 hinge domain, and IgG4 hinge domain, or a functional variant thereof. In some embodiments, the hinge polypeptide sequence contains one or more COX motifs. In some embodiments, the hinge polypeptide sequence contains one or more CPPC motifs (SEQ ID NO: 124).

Hinge polypeptide sequences can also be derived from a CD8α hinge domain, a CD28 hinge domain, a CD152 hinge domain, a PD-1 hinge domain, a CTLA4 hinge domain, an OX40 hinge domain, and functional variants thereof. In some embodiments, the hinge domain includes a hinge polypeptide sequence derived from a CD8 a hinge domain or a functional variant thereof. In some embodiments, the hinge domain includes a hinge polypeptide sequence derived from a CD28 hinge domain or a functional variant thereof. In some embodiments, the hinge domain includes a hinge polypeptide sequence derived from an OX40 hinge domain or a functional variant thereof. In some embodiments, the hinge domain includes a hinge polypeptide sequence derived from an IgG4 hinge domain or a functional variant thereof.

The Fn Notch linking polypeptide is derived from the Robol JMD, which contains a fibronectin repeat (Fn) domain, with a short polypeptide sequence between the Fn repeats and the TMD. The Fn Notch linking polypeptide does not contain a Notch negative regulatory region (NRR), or the Notch HD domain. The Fn linking polypeptide can contain 1, 2, 3, 4, or 5 Fn repeats. In some embodiments, the chimeric receptor comprises a Fn linking polypeptide having about 1 to about 5 Fn repeats, about 1 to about 3 Fn repeats, or about 2 to about 3 Fn repeats. The short polypeptide sequence between the Fn repeats and the TMD can be from about 2 to about 30 amino acid residues. In some embodiments, the short polypeptide sequence can be between about 5 and about 20 amino acids, of any sequence. In some embodiments, the short polypeptide sequence can be between about 5 and about 20 naturally-occurring amino acids, of any sequence. In some embodiments, the short polypeptide sequence can be between about 5 and about 20 amino acids, of any sequence but having no more than one proline. In some embodiments, the short polypeptide sequence can be between about 5 and about 20 amino acids, and about 50% or more of the amino acids are glycine. In some embodiments, the short polypeptide sequence can be between about 5 and about 20 amino acids, where the amino acids are selected from glycine, serine, threonine, and alanine. In some embodiments, the length and amino acid composition of the Fn linking polypeptide sequence can be varied to alter the orientation and/or proximity of the ECD and the TMD relative to one another to achieve a desired activity of the chimeric polypeptide of the disclosure.

Stop-Transfer Sequence

In some embodiments, the priming receptor further comprises a stop-transfer sequence (STS) in between the transmembrane domain and the intracellular domains. The STS comprises a charged, lipophobic sequence. Without being bound by any theory, the STS serves as a membrane anchor, and is believed to prevent passage of the intracellular domain into the plasma membrane. The use of STS domains in priming receptors is described in WO2021061872, hereby incorporated by reference in its entirety. Non-limiting exemplary STS sequences include APLP1, APLP2, APP, TGBR3, CSF1R, CXCL16, CX3CL1, DAG1, DCC, DNER, DSG2, CDH1, GHR, HLA-A, IFNAR2, IGF1R, IL1R1, ERN2, KCNE1, KCNE2, CHL1, LRP1, LRP2, LRP18, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKTFD1, NECTIN1, KL, IL6R, EFNB1, CD44, CLSTN1, LRP8, PCDHGC3, NRG1, LRP1B, JAG2, EFNB2, DLL1, CLSTN2, EPCAM, ErbB4, KCNE3, CDH2, NRG2, PTPRK, BTC, EPHA4, IL1R2, KCNE4, SCN2B, Nradd, PTPRM, Notch1, Notch2, Notch3, and Notch4 STS sequences. In some embodiments, the STS is heterologous to the transmembrane domain. In some embodiments, the STS is homologous to the transmembrane domain. STS sequences are described in WO2021061872, hereby incorporated by reference in its entirety.

Chimeric Antigen Receptors

The recombinant CAR may be a human CAR, comprising fully human sequences, e.g., natural human sequences.

In some embodiments, the recombinant receptor such as a CAR, such as the antibody portion thereof, further includes a spacer, which may be or include at least a portion of an immunoglobulin constant region or variant or modified version thereof, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014031687. In some embodiments, the constant region or portion is of IgD.

The antigen recognition domain of a receptor such as a CAR can be linked to one or more intracellular signaling components, such as signaling components that mimic activation through an antigen receptor complex, such as a TCR complex, in the case of a CAR, and/or signal via another cell surface receptor. Thus, in some embodiments, the extracellular binding component (e.g., ligand-binding or antigen-binding domain) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, and/or CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the receptor.

The receptor, e.g., the CAR, can include at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the extracellular domain is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor-gamma, CD8, CD4, CD25, or CD16. For example, in some aspects, the CAR includes a chimeric molecule between CD3-zeta or Fc receptor-gamma and CD8, CD4, CD25 or CD16.

In some embodiments, upon ligation of the CAR, the cytoplasmic domain or intracellular signaling domain of the receptor activates at least one of the normal effector functions or responses of the immune cell, e.g., T cell engineered to express the receptor. For example, in some contexts, the receptor induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the receptor. In other embodiments, the receptor does not include a component for generating a costimulatory signal. In some aspects, an additional receptor is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the receptor includes one or both of such signaling components.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the receptor includes a signaling domain and/or transmembrane portion of a costimulatory receptor, such as CD28, 4-1BB, OX40, DAP10, and ICOS. In some aspects, the same receptor includes both the activating and costimulatory components.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the receptor encompasses one or more, e.g., two or more, costimulatory domains and an activation domain, e.g., primary activation domain, in the cytoplasmic portion. Exemplary receptors include intracellular components of CD3-zeta, CD28, and 4-1BB.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a nerve growth factor receptor (NGFR), or epidermal growth factor receptor (e.g., tEGFR). In some embodiments, the nucleic acid encoding the marker is operably linked to a polynucleotide encoding for a linker sequence, such as a cleavable linker sequence or a ribosomal skip sequence, e.g., T2A. See WO2014031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published patent application No. WO2014031687. For example, the marker can be a truncated EGFR (tEGFR) that is, optionally, linked to a linker sequence, such as a T2A ribosomal skip sequence.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

The CAR may comprise one or modified synthetic amino acids in place of one or more naturally-occurring amino acids. Exemplary modified amino acids include, but are not limited to, aminocyclohexane carboxylic acid, norleucine, α-amino n-decanoic acid, homoserine, S-acetylaminomethylcysteine, trans-3- and trans-4-hydroxyproline, 4-aminophenylalanine, 4-nitrophenylalanine, 4-chlorophenylalanine, 4-carboxyphenylalanine, (3-phenylserine (3-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,γ-diaminopropionic acid, homophenylalanine, and α-tertbutylglycine.

In some cases, CARs are referred to as first, second, and/or third generation CARs. In some aspects, a first generation CAR is one that solely provides a CD3-chain induced signal upon antigen binding; in some aspects, a second-generation CAR is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular portion containing an antibody or fragment described herein. In some aspects, the chimeric antigen receptor includes an extracellular portion containing an antibody or fragment described herein and an intracellular signaling domain. In some embodiments, an antibody or fragment includes an scFv or a single-domain VH antibody and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 41BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the CAR, is a transmembrane domain of human CD28 or variant thereof, e.g., a 27-amino acid transmembrane domain of a human CD28 (Accession No.: P10747.1).

In some embodiments, the chimeric antigen receptor contains an intracellular domain of a T cell costimulatory molecule. In some aspects, the T cell costimulatory molecule is CD28 or 4-1BB.

In some embodiments, the intracellular signaling domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular domain comprises an intracellular costimulatory signaling domain of 41BB or functional variant or portion thereof, such as a 42-amino acid cytoplasmic domain of a human 4-1BB (Accession No. Q07011.1) or functional variant or portion thereof.

In some embodiments, the intracellular signaling domain comprises a human CD3 zeta stimulatory signaling domain or functional variant thereof, such as a 112 AA cytoplasmic domain of isoform 3 of human CD3.zeta. (Accession No.: P20963.2) or a CD3 zeta signaling domain as described in U.S. Pat. No. 7,446,190 or U.S. Pat. No. 8,911,993.

In some aspects, the spacer contains only a hinge region of an IgG, such as only a hinge of IgG4 or IgG1. In other embodiments, the spacer is an Ig hinge, e.g., and IgG4 hinge, linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

For example, in some embodiments, the CAR includes an antibody or fragment thereof, including single chain antibodies (sdAbs, e.g. containing only the VH region) and scFvs, described herein, a spacer such as any of the Ig-hinge containing spacers, a CD28 transmembrane domain, a CD28 intracellular signaling domain, and a CD3 zeta signaling domain. In some embodiments, the CAR includes an antibody or fragment, including sdAbs and scFvs described herein, a spacer such as any of the Ig-hinge containing spacers, a CD28 transmembrane domain, a CD28 intracellular signaling domain, and a CD3 zeta signaling domain.

Chimeric antigen receptor (CAR) T cells are T cells that have been genetically engineered to produce an artificial T-cell receptor for use in immunotherapy. Chimeric antigen receptors are receptor proteins that have been engineered to confer T cells with the ability to target a specific protein. The genetic modification of lymphocytes (e.g. T cells) by incorporation of, for example, CARs, and administration of the engineered cells to a subject is an example of “adoptive cell therapy”. As used herein, the term “adoptive cell therapy” refers to cell-based immunotherapy for transfusion of autologous or allogeneic lymphocytes, referred to as T cells or B cells. In this CAR therapy approach, cells are expanded and cultured ex vivo and genetically modified, prior to transfusion.

The expression of CARs allows the engineered T-cells to target and bind specific proteins, for example, tumor antigens. In CAR therapy, T-cells are harvested from a subject—they can be autologous T-cells from the subject own blood or from a donor that will not be receiving the CAR therapy. Once isolated, the T-cells are genetically modified with a CAR, expanded ex vivo, and administered to the subject (i.e. patient) by, e.g. infusion.

The CARs may be introduced into the T-cells using, for example, a site-specific technique. With site specific integration of the transgenes (e.g. CARs), the transgenes may be targeted to a safe harbor locus or TRAC. Examples of site-specific techniques for integration into the safe harbor loci include, without limitation, homology-dependent engineering using nucleases and homology independent targeted insertion using Cas9.

The engineered CAR T cells have applications to immune-oncology. The CAR, for example, can be selected to target a specific tumor antigen. Examples of cancers that can be effectively targeted using CAR T cells are blood cancers. In some embodiments, CAR T cell therapy can be used to treat solid tumors.

Recombinant Cells

Also provided herein is a recombinant primary immune cell comprising at least one DNA template non-virally inserted into a target region of the genome of the cell, wherein the size of the DNA template is greater than or equal to about 4.5 or 5 kilobase pairs (kb).

A cell comprising an insert at a target locus or safe harbor site as described in the present disclosure can be referred to as an engineered cell. In some embodiments, the immune cell is any cell that can give rise to a pluripotent immune cell. In some embodiments, the immune cell can be an induced pluripotent stem cell (iPSC) or a human pluripotent stem cell (HSPC). In some embodiments, the immune cell comprises primary hematopoietic cells or primary hematopoietic stem cells. In some embodiments, that engineered cell is a stem cell, a human cell, a primary cell, an hematopoietic cell, an adaptive immune cell, an innate immune cell, a natural killer (NK) cell, a T cell, a CD8+ cell, a CD4+ cell, or a T cell progenitor. In some embodiments, the immune cells are T cells. In some embodiments, the T cells are regulatory T cells, effector T cells, or naïve T cells. In some embodiments, the T cells are CD8⁺ T cells. In some embodiments, the T cells are CD4⁺ T cells. In some embodiments, the T cells are CD4⁺CD8⁺ T cells.

In some embodiments, the engineered cell is a stem cell, a human cell, a primary cell, an hematopoietic cell, an adaptive immune cell, an innate immune cell, a T cell or a T cell progenitor. Non-limiting examples of immune cells that are contemplated in the present disclosure include T cell, B cell, natural killer (NK) cell, NKT/iNKT cell, macrophage, myeloid cell, and dendritic cells. Non-limiting examples of stem cells that are contemplated in the present disclosure include pluripotent stem cells (PSCs), embryonic stem cells (ESCs), induced pluripotent stem cells (iPSCs), embryo-derived embryonic stem cells obtained by nuclear transfer (ntES; nuclear transfer ES), male germline stem cells (GS cells), embryonic germ cells (EG cells), hematopoietic stem/progenitor stem cells (HSPCs), somatic stem cells (adult stem cells), hemangioblasts, neural stem cells, mesenchymal stem cells and stem cells of other cells (including osteocyte, chondrocyte, myocyte, cardiac myocyte, neuron, tendon cell, adipocyte, pancreocyte, hepatocyte, nephrocyte and follicle cells and so on). In some embodiments, the engineered cells is a T cell, NK cells, iPSC, and HSPC. In some embodiments, the engineered cells used in the present disclosure are human cell lines grown in vitro (e.g. deliberately immortalized cell lines, cancer cell lines, etc.).

Also provided herein are populations of cells comprising a plurality of the primary immune cell. In some embodiments, the genome of at least 20%, 30%, 40%, 50%, 60%, 70%, 80%, 90%, 95%, 99% or greater of the cells comprises a targeted insertion of a heterologous DNA template, wherein the DNA template is at least about 5 kb in size.

Methods of Editing Cells

The terms “gene editing” or “genome editing”, as used herein, refer to a type of genetic manipulation in which DNA is inserted, replaced, or removed from the genome using artificially manipulated nucleases or “molecular scissors”. It is a useful tool for elucidating the function and effect of sequence-specific genes or proteins or altering cell behavior (e.g. for therapeutic purposes).

Currently available genome editing tools include zinc finger nucleases (ZFN) and transcription activator-like effector nucleases (TALENs) to incorporate genes at safe harbor loci (e.g. the adeno-associated virus integration site 1 (AAVS1) safe harbor locus). The DICE (dual integrase cassette exchange) system utilizing phiC31 integrase and Bxbl integrase is a tool for target integration. Additionally, clustered regularly interspaced short palindromic repeat/Cas9 (CRISPR/Cas9) techniques can be used for targeted gene insertion.

Site specific gene editing approaches can include homology dependent mechanisms or homology independent mechanisms.

All methods known in the art for targeted insertion of gene sequences are contemplated in the methods described herein to insert constructs at gene targets or safe harbor loci.

Provided herein are methods of inserting nucleotide sequences greater than about 5 kilobases in length into the genome of a cell, in the absence of a viral vector. In some embodiments, the nucleotide sequence greater than about 5 kilobase in length can be inserted into the genome of a primary immune cell, in the absence of a viral vector

Integration of large nucleic acids, for example nucleic acids greater than 5 kilobase in size, into cells, can be limited by low efficiency of integration, off-target effects and/or loss of cell viability. Described herein are methods and compositions for achieving integration of a nucleotide sequence, for example, a nucleotide sequence greater than about 5 kilobases in size, into the genome of a cell. In some methods the efficiency of integration is increased, off-target effects are reduced and/or loss of cell viability is reduced.

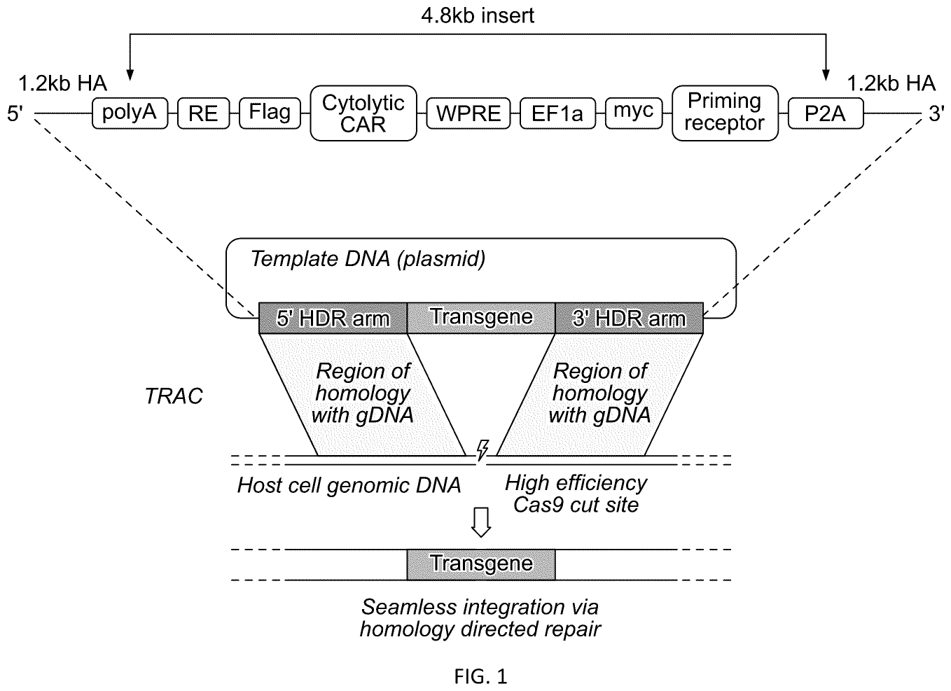

FIG. 1 shows an exemplary plasmid that encodes the priming receptor and the CAR, and a schematic for its incorporation into the genome of an immune cell. The plasmid is introduced into an immune cell with a nuclease, such as a CRISPR-associated system (Cas). The nuclease can be introduced in a ribonucleoprotein format with a guide RNA (gRNA) that targets a specific site on the genome of the immune cell. The nuclease cuts the genomic DNA at this specific site. The specific site may be a portion of the genome that encodes an endogenous immune cell receptor. Thus, cutting the genome at this site will cause the immune cell to no longer express an endogenous immune cell receptor.

As shown in FIG. 1, the plasmid may include 5′ and 3′ homology-directed repair arms complementary to sequences at a specific site on the genome of the immune cell. The complementary sequences are on either side of the site cut by the nuclease, which allows the plasmid to be incorporated at a specified insertion site on the immune cell's genome. Once the plasmid is incorporated, the cell will express the priming receptor. However, as explained, the design of the transgene cassette ensures that non-virally delivered circuit receptors do not express CAR until the priming receptor binds to its cognate ligand and releases the cleavable transcription factor.

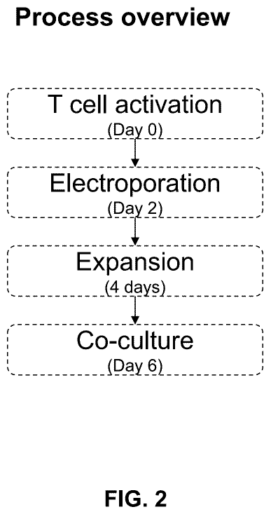

FIG. 2 shows a schematic for an exemplary method of producing an engineered immune cell of the present disclosure. Initially, a T cell is activated. The T cell may be obtained from a patient. Thus, the present disclosure provides methods in which immune cells, such as T cells, are harvested from a patient. Then, the plasmid that encodes the CAR and priming receptor are introduced into a T cell. Advantageously, the plasmids of the present disclosure can be introduced using electroporation. When introducing the plasmid via electroporation, the nuclease may also be introduced. By using electroporation, methods of the present disclosure avoid the use of viral vectors for introducing transgenes, which is a known bottleneck in immune cell engineering. The T cells are then expanded and co-cultured to create a sufficient quantity of engineered immune cells to be used as a therapeutic treatment.

Methods for editing the genome of a cell can include a) providing a Cas9 ribonucleoprotein complex (RNP)-DNA template complex comprising: (i) the RNP, wherein the RNP comprises a Cas9 nuclease domain and a guide RNA, wherein the guide RNA specifically hybridizes to a target region of the genome of the cell, and wherein the Cas9 nuclease domain cleaves the target region to create an insertion site in the genome of the cell; and (ii) a double-stranded or single-stranded DNA template, wherein the size of the DNA template is greater than about 200 nucleotides, wherein the 5′ and 3′ ends of the DNA template comprise nucleotide sequences that are homologous to genomic sequences flanking the insertion site, and wherein the molar ratio of RNP to DNA template in the complex is from about 3:1 to about 100:1; and b) introducing the RNP-DNA template complex into the cell.

In some embodiments, the methods described herein provide an efficiency of delivery of the RNP-DNA template complex of at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the efficiency is determined with respect to cells that are viable after introducing the RNP-DNA template into the cell. In some cases, the efficiency is determined with respect to the total number of cells (viable or non-viable) in which the RNP-DNA template is introduced into the cell.

As another example, the efficiency of delivery can be determined by quantifying the number of genome edited cells in a population of cells (as compared to total cells or total viable cells obtained after the introducing step). Various methods for quantifying genome editing can be utilized. These methods include, but are not limited to, the use of a mismatch-specific nuclease, such as T7 endonuclease I; sequencing of one or more target loci (e.g., by sanger sequencing of cloned target locus amplification fragments); and high-throughput deep sequencing.

In some embodiments, loss of cell viability is reduced as compared to loss of cell viability after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages. In some embodiments, off-target effects of integration are reduced as compared to off-target integration after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages.

In some cases, the methods described herein provide for high cell viability of cells to which the RNP-DNA template has been introduced. In some cases, the viability of the cells to which the RNP-DNA template has been introduced is at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the viability of the cells to which the RNP-DNA template has been introduced is from about 20% to about 99%, from about 30% to about 90%, from about 35% to about 85% or 90% or higher, from about 40% to about 85% or 90% or higher, from about 50% to about 85% or 90% or higher, from about 50% to about 85% or 90% or higher, from about 60% to about 85% or 90% or higher, or from about 70% to about 85% or 90% or higher.

In the methods provided herein, the molar ratio of RNP to DNA template can be from about 3:1 to about 100:1. For example, the molar ratio can be from about 5:1 to 10:1, from about 5:1 to about 15:1, 5:1 to about 20:1; 5:1 to about 25:1; from about 8:1 to about 12:1; from about 8:1 to about 15:1, from about 8:1 to about 20:1, or from about 8:1 to about 25:1.

In some embodiments, the DNA template is at a concentration of about 2.5 pM to about 25 pM. For example, the concentration of DNA template can be about 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25 pM or any concentration in between these concentrations.

In some embodiments, the size or length of the DNA template is greater than about 4.5 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, or 10 kb or any size of DNA template in between these sizes. For example, the size of the DNA template can be about 4.5 kb to about 10 kb, about 5 kb to about 10 kb, about 5 kb to about 9 kb, about 5 kb to about 8 kb, about 5 kb to about 7 kb, about 5 kb to about 6 kb, about kb 6 to about 10 kb, about 6 kb to about 9 kb, about 6 kb to about 8 kb, about 6 kb to about 7 kb, about 7 kb to about 10 kb, about 7 kb to about 9 kb, about 7 kb to about 8 kb, about 8 kb to about 10 kb, about 8 kb to about 9 kb, or about 9 kb to about 10 kb.

In some embodiments, the amount of DNA template is about 1 μg to about 10 μg. For example, the amount of DNA template can be about 1 μg to about 2 μg, about 1 μg to about 3 μg, about 1 μg to about 4 μg, about 1 μg to about 5 μg, about 1 μg to about 6 μg, about 1 μg to about 7 μg, about 1 μg to about 8 μg, about 1 μg to about 9 μg, about 1 μg to about 10 μg. In some embodiments the amount of DNA template is about 2 μg to about 3 μg, about 2 μg to about 4 μg, about 2 μg to about 5 μg, about 2 μg to about 6 μg, about 2 μg to about 7 μg, about 2 μg to about 8 μg, about 2 μg to about 9 μg, or 2 μg to about 10 pg. In some embodiments the amount of DNA template is about 3 μg to about 4 μg, about 3 μg to about 5 μg, about 3 μg to about 6 μg, about 3 μg to about 7 μg, about 3 μg to about 8 μg, about 3 μg to about 9 μg, or about 3 μg to about 10 μg. In some embodiments, the amount of DNA template is about 4 μg to about 5 μg, about 4 μg to about 6 μg, about 4 μg to about 7 μg, about 4 μg to about 8 μg, about 4 μg to about 9 μg, or about 4 μg to about 10 pg. In some embodiments, the amount of DNA template is about 5 μg to about 6 μg, about 5 μg to about 7 μg, about 5 μg to about 8 μg, about 5 μg to about 9 μg, or about 5 μg to about 10 μg. In some embodiments, the amount of DNA template is about 6 μg to about 7 μg, about 6 μg to about 8 μg, about 6 μg to about 9 μg, or about 6 μg to about 10 μg. In some embodiments, the amount of DNA template is about 7 μg to about 8 μg, about 7 μg to about 9 μg, or about 7 μg to about 10 μg. In some embodiments, the amount of DNA template is about 8 μg to about 9 μg, or about 8 μg to about 10 μg. In some embodiments, the amount of DNA template is about 9 μg to about 10 μg.

In some cases, the size of the DNA template is large enough and in sufficient quantity to be lethal as naked DNA. In some embodiments, the DNA template encodes a heterologous protein or a fragment thereof. In some embodiments, the DNA template encodes at least one gene. In some embodiments, the DNA template encodes at least two genes. In some embodiments, the DNA template encodes one, two, three, four, five, six, seven, eight, nine, ten, or more genes.

In some embodiments, the DNA template includes regulatory sequences, for example, a promoter sequence and/or an enhancer sequence to regulate expression of the heterologous protein or fragment thereof after insertion into the genome of a cell.

In some cases, the DNA template is a linear DNA template. In some cases, the DNA template is a single-stranded DNA template. In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA.

In some cases, the RNP-DNA template complex is formed by incubating the RNP with the DNA template for less than about one minute to about thirty minutes, at a temperature of about 20° C. to about 25° C. For example, the RNP can be incubated with the DNA template for about 5 seconds, 10 seconds, 15 seconds, 20 seconds, 25 seconds, 30 seconds, 35 seconds, 40 seconds, 45 seconds, 50 seconds, 55 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes or 30 minutes or any amount of time in between these times, at a temperature of about 20° C., 21° C., 22° C., 23° C., 24° C. or 25° C. In another example, the RNP can be incubated with the DNA template for less than about one minute to about one minute, for less than about one minute to about 5 minutes, for less than about 1 minute to about 10 minutes, for about 5 minutes to 10 minutes, for about 5 minutes to 15 minutes, for about 10 to about 15 minutes, for about 10 minutes to about 20 minutes, or for about 10 minutes to about 30 minutes, at a temperature of about 20° C. to about 25° C. In some embodiments, the RNP-DNA template complex and the cell are mixed prior to introducing the RNP-DNA template complex into the cell.

In some embodiments introducing the RNP-DNA template complex comprises electroporation. Methods, compositions, and devices for electroporating cells to introduce a RNP-DNA template complex can include those described in the examples herein. Additional or alternative methods, compositions, and devices for electroporating cells to introduce a RNP-DNA template complex can include those described in WO/2006/001614 or Kim, J. A. et al. Biosens. Bioelectron. 23, 1353-1360 (2008). Additional or alternative methods, compositions, and devices for electroporating cells to introduce a RNP-DNA template complex can include those described in U.S. Patent Appl. Pub. Nos. 2006/0094095; 2005/0064596; or 2006/0087522. Additional or alternative methods, compositions, and devices for electroporating cells to introduce a RNP-DNA template complex can include those described in Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842, all of which are hereby incorporated by reference. Additional or alternative methods, compositions, and devices for electroporating cells to introduce a RNP-DNA template complex can include those described in Geng, T. et al.. J. Control Release 144, 91-100 (2010); and Wang, J., et al. Lab. Chip 10, 2057-2061 (2010), all of which are hereby incorporated by reference.

In some embodiments, the Cas9 protein can be in an active endonuclease form, such that when bound to target nucleic acid as part of a complex with a guide RNA or part of a complex with a DNA template, a double strand break is introduced into the target nucleic acid. The double strand break can be repaired by NHEJ to introduce random mutations, or HDR to introduce specific mutations. Various Cas9 nucleases can be utilized in the methods described herein. For example, a Cas9 nuclease that requires an NGG protospacer adjacent motif (PAM) immediately 3′ of the region targeted by the guide RNA can be utilized. Such Cas9 nucleases can be targeted to any region of a genome that contains an NGG sequence. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to, CFP1, those described in Nature Methods 10, 1116-1121 (2013), and those described in Zetsche et al., Cell, Volume 163, Issue 3, p759-771, 22 Oct. 2015, both of which are hereby incorporated by reference.

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation.

In some embodiments, the RNP comprises a Cas9 nuclease. In some embodiments, the RNP comprises a Cas9 nickase. In some embodiments, the RNP-DNA template complex comprises at least two structurally different RNP complexes. In some embodiments, the at least two structurally different RNP complexes contain structurally different Cas9 nuclease domains In some embodiments, the at least two structurally different RNP complexes contain structurally different guide RNAs. In some embodiments, wherein the at least two structurally different RNP complexes contain structurally different guide RNAs, each of the structurally different RNP complexes comprises a Cas9 nickase, and the structurally different guide RNAs hybridize to opposite strands of the target region.

In some cases, a plurality of RNP-DNA templates comprising structurally different ribonucleoprotein complexes is introduced into the cell. For example a Cas9 protein can be complexed with a plurality (e.g., 2, 3, 4, 5, or more, e.g., 2-10, 5-100, 20-100) of structurally different guide RNAs to target insertion of a DNA template at a plurality of structurally different target genomic regions.

In the methods and compositions provided herein, cells include, but are not limited to, eukaryotic cells, prokaryotic cells, animal cells, plant cells, fungal cells and the like. Optionally, the cell is a mammalian cell, for example, a human cell. The cell can be in vitro, ex vivo or in vivo. The cell can also be a primary cell, a germ cell, a stem cell or a precursor cell. The precursor cell can be, for example, a pluripotent stem cell, or a hematopoietic stem cell. In some embodiments, the cell is a primary hematopoietic cell or a primary hematopoietic stem cell. In some embodiments, the primary hematopoietic cell is an immune cell. In some embodiments, the immune cell is a T cell. In some embodiments, the T cell is a regulatory T cell, an effector T cell, or a naïve T cell. In some embodiments, the T cell is a CD4⁺ T cell. In some embodiments, the T cell is a CD8⁺ T cell. In some embodiments, the T cell is a CD4⁺CD8⁺ T cell. In some embodiments, the T cell is a CD4⁻CD8⁻ T cell. Populations of any of the cells modified by any of the methods described herein are also provided. In some embodiments, the methods further comprise expanding the population of modified cells.

In some cases, the cells are removed from a subject, modified using any of the methods described herein and administered to the patient. In other cases, any of the constructs described herein is delivered to the patient in vivo. See, for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017), both of which are hereby incorporated by reference.

In some embodiments, the RNP-DNA template complex is introduced into about 1×10⁵ to about 2×10⁶ cells. For example, the RNP-DNA template complex can be introduced into about 1×10⁵ to about 5×10⁵ cells, about 1×10⁵ to about 1×10⁶, 1×10⁵ to about 1.5×10⁶, 1×10⁵ to about 2×10⁶, about 1×10⁶ to about 1.5×10⁶ cells or about 1×10⁶ to about 2×10⁶.

In some cases, the methods and compositions described herein can be used for generation, modification, use, or control of recombinant T cells, such as chimeric antigen receptor T cells (CAR T cells). Such CAR T cells can be used to treat or prevent cancer, an infectious disease, or autoimmune disease in a subject. For example, in some embodiments, one or more gene products are inserted or knocked-in to a T cell to express a heterologous protein (e.g., a chimeric antigen receptor (CAR) or a priming receptor).

Insertion Sites

Methods for editing the genome of a T cell, specifically, include a method of editing the genome of a human T cell comprise inserting a nucleic acid sequence or construct into a target region in exon 1 of the TCR-α subunit (TRAC) gene in the human T cell. In some embodiments, the target region is in exon 1 of the constant domain of TRAC gene. In other embodiments, the target region is in exon 1, exon 2 or exon 3, prior to the start of the sequence encoding the TCR-α transmembrane domain.

Methods for editing the genome of a T cell also include a method of editing the genome of a human T cell comprise inserting a nucleic acid sequence or construct into a target region in exon 1 of a TCR-β subunit (TRBC) gene in the human T cell. In some embodiments, the target region is in exon 1 of the TRBC1 or TRBC2 gene.

Methods for editing the genome of a T cell, specifically, include a method of editing the genome of a human T cell comprise inserting a nucleic acid sequence or construct into a target region of a genomic safe harbor (GSH).

Gene editing therapies include, for example, vector integration and site specific integration. Site-specific integration is a promising alternative to random integration of viral vectors, as it mitigates the risks of insertional mutagenesis or insertional oncogenesis (Kolb et al. Trends Biotechnol. 2005 23:399-406; Porteus et al. Nat Biotechnol. 2005 23:967-973; Paques et al. Curr Gen Ther. 2007 7:49-66). However, site specific integration continues to face challenges such as poor knock-in efficiency, risk of insertional oncogenesis, unstable and/or anomalous expression of adjacent genes or the transgene, low accessibility (e.g. within 20 kB of adjacent genes), etc. These challenges can be addressed, in part, through the identification and use of safe harbor loci or safe harbor sites (SHS), which are sites in which genes or genetic elements can be incorporated without disruption to expression or regulation of adjacent genes.

The most widely used of the putative human safe harbor sites is the AAVS1 site on chromosome 19q, which was initially identified as a site for recurrent adenoassociated virus insertion. Other potential SHS have been identified on the basis of homology, with sites first identified in other species (e.g., the human homolog of the permissive murine Rosa26 locus) or among the growing number of human genes that appear non-essential under some circumstances. One putative SHS of this type is the CCR5 chemokine receptor gene, which, when disrupted, confers resistance to human immunodeficiency virus infection. Additional potential genomic SHS have been identified in human and other cell types on the basis of viral integration site mapping or gene-trap analyses, as was the original murine Rosa26 locus. The three top SHS, AAVS1, CCR5, and Rosa26, are in close proximity to many protein coding genes and regulatory elements. (See Sadelain, M., et al. (2012). Safe harbours for the integration of new DNA in the human genome. Nature reviews Cancer, 12(1), 51-58, the relevant disclosures of which are herein incorporated by reference in their entirety).

The AAVS1 (also known as the PPP1R12C locus) on human chromosome 19 is a known SHS for hosting transgenes (e.g. DNA transgenes) with expected function. It is at position 19q13.42. It has an open chromatin structure and is transcription-competent. The canonical SHS locus for AAVS1 is chr19: 55,625,241-55,629,351. See Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference. An exemplary AAVS1 target gRNA and target sequence are provided below:

AAVS1-gRNA sequence:
(SEQ ID NO: 125)
ggggccactagggacaggatGTTTTAGAGCTAGAAATAGCAAGTTAAAAT
AAGGCTAGTCCGTTATCAACTTGAAAAAGTGGCACCGAGTCGGTGCTTTT
TTT
AAVS1 target sequence:
(SEQ ID NO: 126)
ggggccactagggacaggat

CCR5, which is located on chromosome 3 at position 3p21.31, encodes the major co-receptor for HIV-1. Disruption at this site in the CCR5 gene has been beneficial in HIV/AIDS therapy and prompted the development of zinc-finger nucleases that target its third exon. The canonical SHS locus for CCR5 is chr3: 46,414,443-46,414,942. See Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference.

The mouse Rosa26 locus is particularly useful for genetic modification as it can be targeted with high efficiency and is expressed in most cell types tested. Irion et al. 2007 (“Identification and targeting of the ROSA26 locus in human embryonic stem cells.” Nature biotechnology 25.12 (2007): 1477-1482, the relevant disclosure of which are herein incorporated by reference) identified the human homolog, human ROSA26, in chromosome 3 (position 3p25.3). The canonical SHS locus for human Rosa26 (hRosa26) is chr3: 9,415,082-9,414,043. See Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference.

Additional examples of safe harbor sites are provided in Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference. Examples of additional integration sites are provided in Table 1.

In some embodiments, the safe harbor sites allow for high transgene expression (sufficient to allow for transgene functionality or treatment of a disease of interest) and stable expression of the transgene over several days, weeks or months. In some embodiments, knockout of the gene at the safe harbor locus confers benefit to the function of the cell, or the gene at the safe harbor locus has no known function within the cell. In some embodiments the safe harbor locus results in stable transgene expression in vitro with or without CD3/CD28 stimulation, negligible off-target cleavage as detected by iGuide-Seq or CRISPR-Seq, less off-target cleavage relative to other loci as detected by iGuide-Seq or CRISPR-Seq, negligible transgene-independent cytotoxicity, negligible transgene-independent cytokine expression, negligible transgene-independent chimeric antigen receptor expression, negligible deregulation or silencing of nearby genes, and positioned outside of a cancer-related gene.

As used, a “nearby gene” can refer to a gene that is within about 100 kB, about 125 kB, about 150 kB, about 175 kB, about 200 kB, about 225 kB, about 250 kB, about 275 kB, about 300 kB, about 325 kB, about 350 kB, about 375 kB, about 400 kB, about 425 kB, about 450 kB, about 475 kB, about 500 kB, about 525 kB, about 550 kB away from the safe harbor locus (integration site).

In some embodiments, the present disclosure contemplates inserts that comprise one or more transgenes. The transgene can encode a therapeutic protein, an antibody, a peptide, a suicide gene, an apoptosis gene or any other gene of interest. The transgene integration can result in, for example, enhanced therapeutic properties. These enhanced therapeutic properties, as used herein, refer to an enhanced therapeutic property of a cell when compared to a typical immune cell of the same normal cell type. For example, an NK cell having “enhanced therapeutic properties” has an enhanced, improved, and/or increased treatment outcome when compared to a typical, unmodified and/or naturally occurring NK cell. The therapeutic properties of immune cells can include, but are not limited to, cell transplantation, transport, homing, viability, self-renewal, persistence, immune response control and regulation, survival, and cytotoxicity. The therapeutic properties of immune cells are also manifested by: antigen-targeted receptor expression; HLA presentation or lack thereof; tolerance to the intratumoral microenvironment; induction of bystander immune cells and immune regulation; improved target specificity with reduction; resistance to treatments such as chemotherapy.

As used herein, the term “insert size” refers to the length of the nucleotide sequence being integrated (inserted) at the target locus or safe harbor site. In some embodiments, the insert size comprises at least about 4.5 kilobasepairs (kb) to about 10 kilobasepairs (kb). In some embodiments, the insert size comprises about 5000 nucleotides or more basepairs. In some embodiments, the insert size comprises up to 4.5, 4.8, 5, 6, 7, 8, 9, 10, 11, 12, 13, 14, 15, 16, 17, 18, 19, 20 kbp (kilo basepairs) or the sizes in between. In some embodiments, the insert size is greater than 4.5, 4.8, 5, 6, 7, 8, 9, 10, 11, 12, 13, 14, 15, 16, 17, 18, 19, 20 kbp or the sizes in between. In some embodiments, the insert size is within the range of 4.5-15 kbp or is any number in that range. In some embodiments, the insert size is within the range of 4.8-8.3 kbp or is any number in that range. In some embodiments, the insert size is within the range of 5-8.3 kbp or is any number in that range. In some embodiments, the insert size is within the range of 5-15 kbp or is any number in that range. In some embodiments, the insert size is within the range of 4.5-20 kbp or is any number in that range. In some embodiments, the insert size is 5-10 kbp. In some embodiments, the insert size is 4.5-10, 5-10, 6-10, 7-10, 8-10, 9-10 kbp. In some embodiments, the insert size is 4.5-11, 6-11, 7-11, 8-11, 9-11, or 10-11 kbp. In some embodiments, the insert size is 4.5-12, 6-12, 7-12, 8-12, 9-12, 10-12, or 11-12 kbp. In some embodiments, the insert size is 4.5-13, 6-13, 7-13, 8-13, 9-13, 10-13, 11-13, or 12-13 kbp. In some embodiments, the insert size is 4.5-14, 6-14, 7-14, 8-14, 9-14, 10-14, 11-14, 12-14 or 13-14 kbp. In some embodiments, the insert size is 4.5-15, 6-15, 7-15, 8-15, 9-15, 10-15, 11-15, 12-15, 13-15, or 14-15 kbp. In some embodiments, the insert size is 4.5-16, 6-16, 7-16, 8-16, 9-16, 10-16, 11-16, 12-16, 13-16, 14-16 or 15-16 kbp. In some embodiments, the insert size is 4.5-17, 6-17, 7-17, 8-17, 9-17, 10-17, 11-17, 12-17, 13-17, or 14-17, 15-17 or 16-17 kbp. In some embodiments, the insert size is 4.5-18, 6-18, 7-18, 8-18, 9-18, 10-18, 11-18, 12-18, 13-18, 14-18, 15-18, 16-18 or 17-18 kbp. In some embodiments, the insert size is 4.5-19, 6-19, 7-19, 8-19, 9-19, 10-19, 11-19, 12-19, 13-19, 14-19, 15-19, 16-19, 17-19, or 18-19 kbp. In some embodiments, the insert size is 4.5-20, 6-20, 7-20, 8-20, 9-20, 10-20, 11-20, 12-20, 13-20, 14-20, 15-20, 16-20, 17-20, 18-20, or 19-20 kbp.

The inserts of the present disclosure refer to nucleic acid molecules or polynucleotide inserted at a target locus or safe harbor site. In some embodiments, the nucleotide sequence is a DNA molecule, e.g., genomic DNA, or comprises deoxy-ribonucleotides. In some embodiments, the insert comprises a smaller fragment of DNA, such as a plastid DNA, mitochondrial DNA, or DNA isolated in the form of a plasmid, a fosmid, a cosmid, a bacterial artificial chromosome (BAC), a yeast artificial chromosome (YAC), and/or any other sub-genome segment of DNA. In some embodiments, the insert is an RNA molecule or comprises ribonucleotides. The nucleotides in the insert are contemplated as naturally occurring nucleotides, non-naturally occurring, and modified nucleotides. Nucleotides may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications. The polynucleotides can be in any topological conformation, including single-stranded, double-stranded, partially duplexed, triplexed, hairpinned, circular conformations, and other three-dimension conformations contemplated in the art.

The inserts can have coding and/or non-coding regions. The insert can comprises a non-coding sequence (e.g., control elements, e.g., a promoter sequence). In some embodiments, the insert encodes transcription factors. In some embodiments, the insert encodes an antigen binding receptors such as single receptors, T-cell receptors (TCRs), priming receptors, CARs, mAbs, etc. In some embodiments, the inserts are RNAi molecules, including, but not limited to, miRNAs, siRNA, shRNAs, etc. In some embodiments, the insert is a human sequence. In some embodiments, the insert is chimeric. In some embodiments, the insert is a multi-gene/multi-module therapeutic cassette. A multi-gene/multi-module therapeutic cassette referst to an insert or cassette having one or more than one receptor (e.g., synthetic receptors), other exogenous protein coding sequences, non-coding RNAs, transcriptional regulatory elements, and/or insulator sequences, etc.

In some embodiments, the nucleic acid sequence is inserted into the genome of the T cell via non-viral delivery. In non-viral delivery methods, the nucleic acid can be naked DNA, or in a non-viral plasmid or vector. Non-viral delivery techniques can be site-specific integration techniques, as described herein or known to those of ordinary skill in the art. Examples of site-specific techniques for integration into the safe harbor loci include, without limitation, homology-dependent engineering using nucleases and homology independent targeted insertion using Cas9 or other CRISPR endonucleases.

In some embodiments, the insert is integrated at a safe harbor site by introducing into the engineered cell, (a) a targeted nuclease that cleaves a target region in the safe harbor site to create the insertion site; and (b) the nucleic acid sequence (insert), wherein the insert is incorporated at the insertion site by, e.g., HDR. Examples of non-viral delivery techniques that can be used in the methods of the present disclosure are provided in U.S. application Ser. Nos. 16/568,116 and 16/622,843, the relevant disclosures of which are herein incorporated by reference in their entirety.

CRISPR-Cas Editing

One effective example of gene editing is the CRISPR-Cas approach (e.g. CRISPR-Cas9). This approach incorporates the use of a guide polynucleotide (e.g. guide ribonucleic acid or gRNA) and a cas endonuclease (e.g. Cas9 endonuclease).

As used herein, a polypeptide referred to as a “Cas endonuclease” or having “Cas endonuclease activity” refers to a CRISPR-related (Cas) polypeptide encoded by a Cas gene, wherein a Cas polypeptide is a target DNA sequence that can be cleaved when operably linked to one or more guide polynucleotides (see, e.g., U.S. Pat. No. 8,697,359). Also included in this definition are variants of Cas endonuclease that retain guide polynucleotide-dependent endonuclease activity. The Cas endonuclease used in the donor DNA insertion method detailed herein is an endonuclease that introduces double-strand breaks into DNA at the target site (e.g., within the target locus or at the safe harbor site).

As used herein, the term “guide polynucleotide” relates to a polynucleotide sequence capable of complexing with a Cas endonuclease and allowing the Cas endonuclease to recognize and cleave a DNA target site. The guide polynucleotide can be a single molecule or a double molecule. The guide polynucleotide sequence can be an RNA sequence, a DNA sequence, or a combination thereof (RNA-DNA combination sequence). A guide polynucleotide comprising only ribonucleic acid is also referred to as “guide RNA”. In some embodiments, a polynucleotide donor construct is inserted at a safe harbor locus using a guide RNA (gRNA) in combination with a cas endonuclease (e.g. Cas9 endonuclease).

The guide polynucleotide includes a first nucleotide sequence domain (also referred to as a variable targeting domain or VT domain) that is complementary to a nucleotide sequence in the target DNA, and a second nucleotide that interacts with a Cas endonuclease polypeptide. It can be a double molecule (also referred to as a double-stranded guide polynucleotide) comprising a sequence domain (referred to as a Cas endonuclease recognition domain or CER domain). The CER domain of this double molecule guide polynucleotide comprises two separate molecules that hybridize along the complementary region. The two separate molecules can be RNA sequences, DNA sequences and/or RNA-DNA combination sequences.

Genome editing using CRISPR-Cas approaches relies on the repair of site-specific DNA double-strand breaks (DSBs) induced by the RNA-guided Cas endonuclease (e.g. Cas 9 endonuclease). Homology-directed repair (HDR) of these DSBs enables precise editing of the genome by introducing defined genomic changes, including base substitutions, sequence insertions, and deletions. Conventional HDR-based CRISPR/Cas9 genome-editing involves transfecting cells with Cas9, gRNA and donor DNA containing homologous arms matching the genomic locus of interest.

HITI (homology independent targeted insertion) uses a non-homologous end joining (NHEJ)-based homology-independent strategy and the method can be more efficient than HDR. Guide RNAs (gRNAs) target the insertion site. For HITI, donor plasmids lack homology arms and DSB repair does not occur through the HDR pathway. The donor polynucleotide construct can be engineered to include Cas9 cleavage site(s) flanking the gene or sequence to be inserted. This results in Cas9 cleavage at both the donor plasmid and the genomic target sequence. Both target and donor have blunt ends and the linearized donor DNA plasmid is used by the NHEJ pathway resulting integration into the genomic DSB site. (See, for example, Suzuki, K., et al. (2016). In vivo genome editing via CRISPR/Cas9 mediated homology-independent targeted integration. Nature, 540(7631), 144-149, the relevant disclosures of which are herein incorporated in their entirety).

Methods for conducing gene editing using CRISPR-Cas approaches are known to those of ordinary skill in the art. (See, for example, U.S. aaplication Ser. Nos. 16/312,676, 15/303,722, and 15/628,533, the disclosures of which are herein incorporated by reference in their entirety). Additionally, uses of endonucleases for inserting transgenes into safe harbor loci are described, for example, in U.S. application Ser. No. 13/036,343, the disclosures of which are herein incorporated by reference in their entirety.

The guide RNAs and/or mRNA (or DNA) encoding an endonuclease can be chemically linked to one or more moieties or conjugates that enhance the activity, cellular distribution, or cellular uptake of the oligonucleotide. Non-limiting examples of such moieties include lipid moieties such as a cholesterol moiety, cholic acid, a thioether, a thiocholesterol, an aliphatic chain (e.g., dodecandiol or undecyl residues), a phospholipid, e.g., di-hexadecyl-rac-glycerol or triethylammonium 1,2-di-O-hexadecyl-rac-glycero-3-H-phosphonate, a polyamine or a polyethylene glycol chain, adamantane acetic acid, a palmityl moiety and an octadecylamine or hexylamino-carbonyl-t oxycholesterol moiety. See for example US Patent Publication No. 20180127786, the disclosure of which is herein incorporated by reference in its entirety.

Therapeutic Applications

For therapeutic applications, the engineered cells, populations thereof, or compositions thereof are administered to a subject, generally a mammal, generally a human, in an effective amount.

The engineered cells may be administered to a subject by infusion (e.g., continuous infusion over a period of time) or other modes of administration known to those of ordinary skill in the art.

The engineered cells provided herein not only find use in gene therapy but also in non-pharmaceutical uses such as, e.g., production of animal models and production of recombinant cell lines expressing a protein of interest.

The engineered cells of the present disclosure can be any cell, generally a mammalian cell, generally a human cell that has been modified by integrating a transgene at a safe harbor locus described herein. In some embodiments, the engineered cells are immune cells. In some embodiments, the engineered cells are lymphocytes. In some embodiments, the engineered cells are T cells or T cell precursors.

The engineered cells, compositions and methods of the present disclosure are useful for therapeutic applications such as CAR T cell therapy and TCR T cell therapy. In some embodiments, the insertion of a sequence encoding a transgene within a safe harbor locus maintains the TCR expression relative to instances when there is no insertion and enables transgene expression while maintaining TCR function.

In some embodiments, the present disclosure provides methods of treating a subject in need of treatment by administering to the subject a composition comprising any of the engineered cells described herein. As used, the terms “treat,” “treatment,” and the like refer generally to obtaining a desired pharmacological and/or physiological effect. That effect is preventive in terms of complete or partial prevention of the disease and/or therapeutic in terms of partial or complete cure of the disease and/or adverse effects resulting from the disease. The term “treatment”, as used herein, encompasses any treatment of a disease in a subject (e.g., mammal, e.g., human). Treatment may also refer to the administration of the engineered cells provided herein to a subject that is susceptible to the disease but has not yet been diagnosed as suffering from it, including preventing the disease from occurring; inhibiting disease progression; or reducing the disease (i.e., causing a regression of the disease). Further, treatment may stabilize or reduce undesirable clinical symptoms in subjects (e.g., patients). The cells provided herein populations thereof, or compositions thereof may be administered before, during or after the occurrence of the disease or injury.

In certain embodiments, the subject has a disease, condition, and/or injury that can be treated and/or ameliorated by cell therapy. In some embodiments, the subject in need of cell therapy is a subject having an injury, disease, or condition, thereby causing cell therapy (e.g., therapy in which cellular material is administered to the subject). However, it is contemplated that it is possible to treat, ameliorate and/or reduce the severity of at least one symptom associated with the injury, disease or condition. In certain embodiments, a subject in need of cell therapy includes, but is not limited to, a bone marrow transplant or stem cell transplant candidate, a subject who has received chemotherapy or radiation therapy, a hyperproliferative disease or cancer (e.g., a hematopoietic system), a subject having or at risk of developing a hyperproliferative disease or cancer), a subject having or at risk of developing a tumor (e.g., solid tumor), viral infection or virus. It is also intended to encompass subjects suffering from or at risk of suffering from a disease associated with an infection.

In some embodiments, the present disclosure provides a composition of the present disclosure along with instructions for use. The instructions for use can be present in the kits as a package insert, in the labeling of the container of the kit or components thereof, or can be in digital form (e.g. on a CD-ROM, via a link on the internet). A kit can include one or more of a genome-targeting nucleic acid, a polynucleotide encoding a genome-targeting nucleic acid, a site-directed polypeptide, and/or a polynucleotide encoding a site-directed polypeptide. Additional components within the kits are also contemplated, for example, buffer (such as reconstituting buffer, stabilizing buffer, diluting buffer), and/or one or more control vectors.

Pharmaceutical Compositions

The engineered recombinant cells provided herein can be administered as part of a pharmaceutical compositions. These compositions can comprise, in addition to one or more of the recombinant cells, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes. The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

Various modes of administering the additional therapeutic agents are contemplated herein. In some embodiments, the additional therapeutic agent is administered by any suitable mode of administration. Generally, modes of administration include, without limitation, intravitreal, subretinal, suprachoroidal, intraarterial, intradermal, intramuscular, intraperitoneal, intravenous, nasal, parenteral, topical, pulmonary, and subcutaneous routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

Whether it is a polypeptide, cell, or nucleic acid, or other pharmaceutically useful compound according to the present disclosure that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3_{rd} Ed. (Plenum Press) Vols A and B (1992).

### Example 1: Non-Viral Editing of Immune Cells with Large DNA Inserts

Viral delivery of gene circuits or systems is difficult due to i) size limitations of the expression cassette, ii) transgene layout limitations due to conflicts with viral vector biology, and iii) poor and unpredictable performance of transgenes at random integration sites. The following examples demonstrate delivery of large cassettes that would not be possible with available viral vectors to defined insertion sites, where they have predictable, high performance.

Construct Generation

To generate plasmid constructs for knock-in, synthetic DNA was ordered from Twist, IDT and GENEWIZ and assembled via Gibson Assembly and Golden Gate Assembly. Plasmids contained homology arms homologous to sequences flanking the CRISPR target sites in the genome of 1.2 kb in length.

T Cell Engineering

T-cells were enriched from peripheral blood mononuclear cells (PBMCs) obtained from normal donor Leukopaks (STEMCELL Technologies) using Lymphoprep (STEMCELL Technologies) and the EasySep Human T-Cell Isolation Kit (STEMCELL Technologies). T-cells were subsequently activated with CD3/CD28 Dynabeads at 1:1 bead to cell ratio (ThermoFisher, 40203D) in TexMACS medium (Miltenyi 130-197-196) supplemented with 3% human AB serum (Gemini Bio) and 12.5 ng/ml human IL-7 and IL-15 (Miltenyi premium grade) and cultured at 37° C., 5% CO2 for 48 hours before electroporation.

CRISPR RNP were prepared by combining 120 μM sgRNA (Synthego) targeting DNA sequence GTCAGGGTTCTGGATATCTG (TRAC, SEQ ID NO: 79), 62.5 μM sNLS-SpCas9-sNLS (Aldevron) and P3 buffer (Lonza) at a volume ratio of 5:1:3:6, and incubated for 15 minutes at room temperature. An optimized amount of plasmid DNA, determined by dose titration experiments (ranging from 0.5-3 micrograms) was mixed with 3.5 μl of RNP. T-cells were counted, debeaded, centrifuged at 90×G for 10 minutes and resuspended at 10\hat{ }6 cells/14.5 μl of P3 with supplement added (Lonza). 14.5 μl of T-cell suspension was added to the DNA/RNP mixture, transferred to Lonza 16-well cuvette strip, and pulsed in a Lonza 4D Nucleofector System with code EH-115. Cells were allowed to rest for 15 minutes at room temperature before transfer to 96-well plates (Sarstedt) in TexMACS medium supplemented with 12.5 ng/ml human IL-7 and IL-15 (Miltenyi premium grade).

Transgene expression was detected by staining with anti-Myc antibody (Cell Signaling Technology clone 9B11) and anti-Flag antibody (RnD systems, clone 1042E) and analyzed on an Attune NxT Flow Cytometer. Other antibodies used were live/dead Fixable Near-IR (Thermo Fisher), TCRalpha/beta antibody (BioLegend clone IP26), CD4 antibody (BioLegend clone RPA-T4), CD8 antibody (BioLegend clone SK1).

Priming Receptor Induction

To assess functional activity of transgene (ie. synthetic circuit), edited T cells were co-cultured with target cell line expressing priming antigen at 1:1 E:T ratio, and incubated for 24 hrs. T cells were harvested and stained with anti-Myc and anti-Flag antibodies to assess for Prime Receptor and CAR expression, respectively.

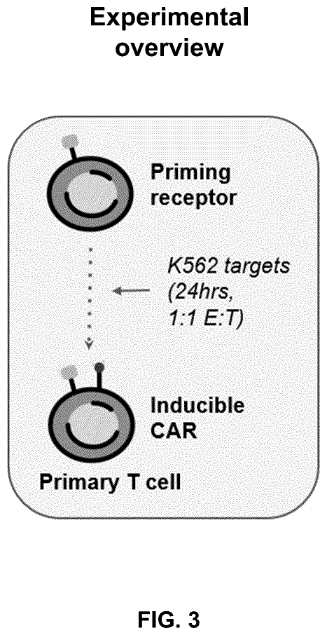

FIG. 3 provides an overview for an experimental protocol used to show the fidelity of a synthetic circuit introduced to the TRAC locus of T cells using the tailored transgene cassette as shown in FIG. 1 and a non-viral gene editing technology. The T cells include a FLAG tag that is co-expressed with the CAR and a myc-tag that is co-expressed with the priming receptor (primeR). The engineered immune cells expressing the transgene insert in the TRAC locus were prepared, and initially expressed the priming receptor. The engineered immune cells were then incubated for 24 hours with K562 target cells expressing the priming antigen at 1:1 E:T ratio. These target cells express the cognate ligand for the priming receptor. T cells were harvested and stained with anti-Myc and anti-Flag antibodies to assess priming receptor and CAR expression, respectively. CAR induction on edited T cells is expected in the presence of priming antigen only. Thus, after sufficient incubation with the target cells, the engineered immune cells express the CAR.

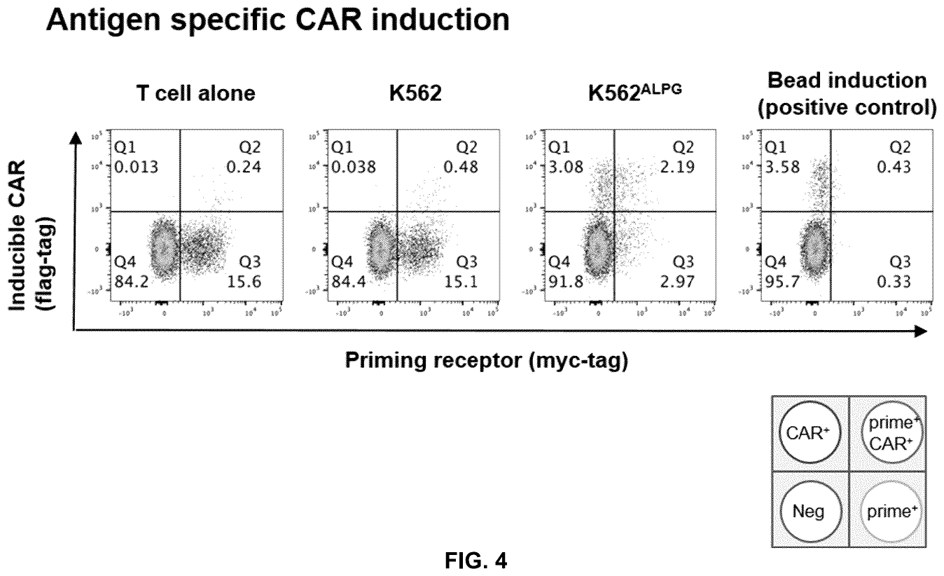

FIG. 4 provides the experimental results. Analysis was readout on an Attune NxT Flow Cytometer. As shown, when the T cells were incubated alone or with the target cell K562 in the absence of the priming receptor ligand, there was very little CAR expression, but high levels of priming receptor expression. This indicates that the priming receptor is specifically expressed and activated as intended. It also indicates that, in the absence of the priming receptor ligand, very few cells express the CAR. Thus, the circuit receptor is not leaky. When the T cells were incubated with the target cells that had the priming receptor cognate protein (K562^{ALPG}), many T cells show expression of the CAR. This indicates that activation of the priming receptor induces expression of the CAR. There is also a corresponding decrease in the expression of the priming receptor as due to cleavage by gamma secretase, which also releases the transcription factor. Thus, CAR induction was only observed in the presence of priming antigen and correlated with loss of priming receptor expression. The obtained results demonstrate that circuit's fidelity is maintained following delivery of the transgene using CRISPR technology.

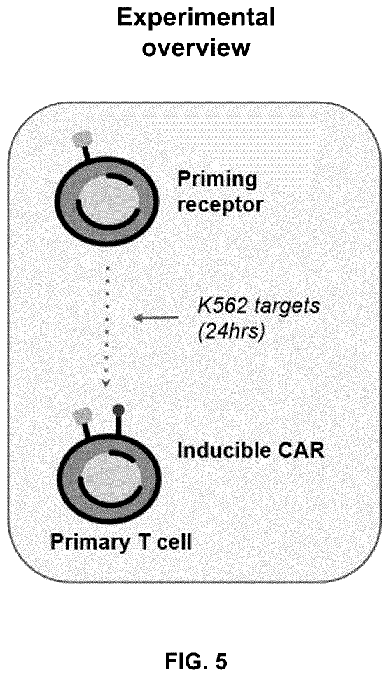

FIG. 5 provides an overview for an experimental protocol used to show the fidelity of a synthetic circuit introduced to T cells using the tailored transgene cassette as shown in FIG. 1 and a non-viral gene editing technology. The T cells include a FLAG tag that is co-expressed with the CAR and a myc-tag that is co-expressed with the priming receptor (primeR). The engineered immune cells were prepared, and initially express the priming receptor. The engineered immune cells were then incubated for 24 hours with target cells expressing the priming antigen at 1:1 E:T ratio. These target cells express the cognate ligand for the priming receptor. T cells were harvested and stained with anti-Myc and anti-Flag antibodies to assess priming receptor and CAR expression, respectively. CAR induction on edited T cells is expected in the presence of priming antigen only. Thus, after sufficient incubation with the target cells, the engineered immune cells express the CAR.

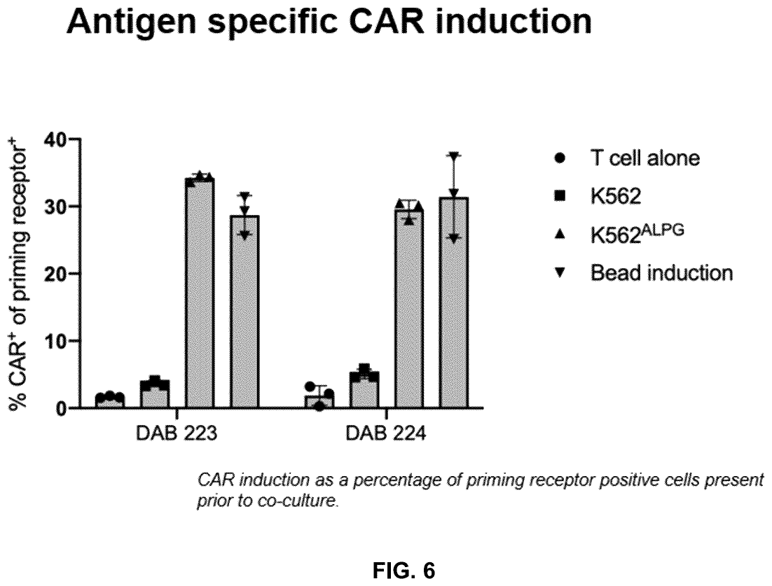

FIG. 6 shows further quantitative data from two donors that show a very small percentage of T cells express the CAR in the absence of the priming receptor activation. A synthetic circuit was delivered to T cells derived from 2 healthy donors using CRISPR technology. The edited T cells were co-cultured with a K562 cell line expressing the cognate priming at 1:1 E:T ratio, a negative control K562 parental cell line, or Myc beads as positive control, and incubated for 24 hrs. T cells were harvested and stained with anti-Myc and anti-Flag antibodies to assess for Prime Receptor and CAR expression, respectively. Analysis was readout on an Attune NxT Flow Cytometer. Over 30% of priming receptor positive T cells, expressed CAR in the presence of target cognate antigen or myc beads control. Minimal CAR expression was observed at the basal level or when cultured with the K562 parental cell line. Taken together, the results demonstrate that knock-in of the synthetic circuit to TRAC locus does not impair its function. This also indicates that the DNA cassette encoding the CAR is not leaky.

### Example 2: Evaluation of Large Circuit Expression and Function Following Non-Viral Insertion

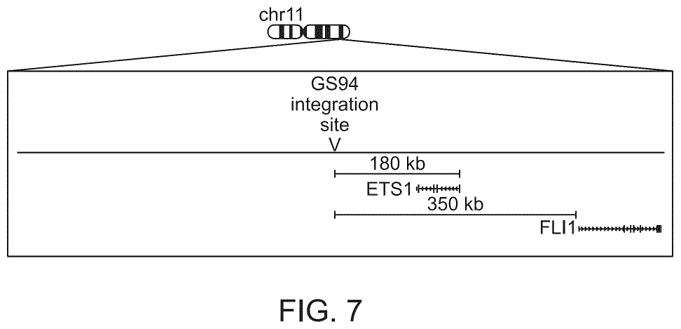

GS94 is a candidate integration site located on chromosome 11's distal q arm. It is within 180-350 kb of the promoters for ETS1 and FLI1 (FIG. 7), however it is considered low-risk for integration vector gene therapy. The circuit expression and function potential of the GS94 gene was evaluated.

Construct Generation

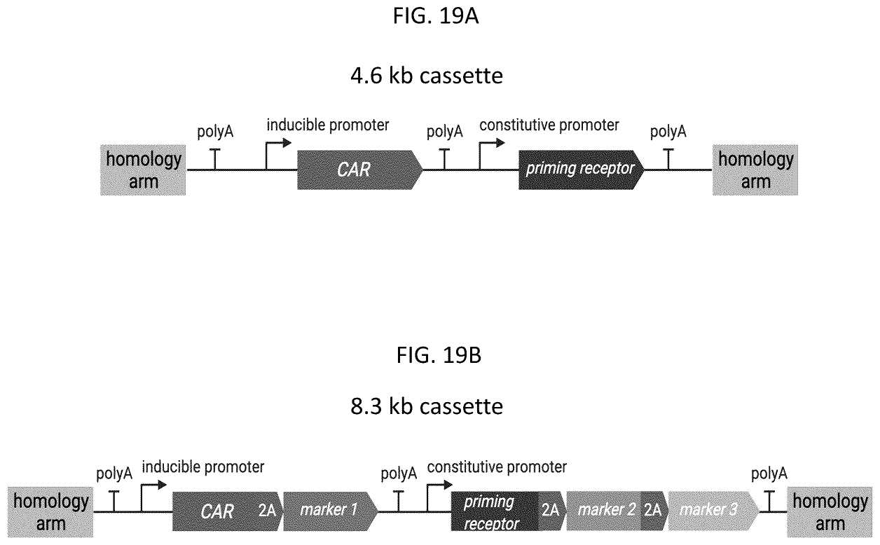

To generate plasmid constructs for knock-in, synthetic DNA was ordered from Twist, IDT and GENEWIZ and assembled via Gibson Assembly and Golden Gate Assembly. Plasmids contained homology arms homologous to sequences flanking the CRISPR target sites in the genome of length 1.2 kb or 450 bp. The circuit cassette was 4558 bp in length. A diagram of the cassette is shown in FIG. 19A.

T cells underwent circuit cassette integration with PrimeR at GS79 (TRAC) integration site, GS94 integration site, and GS102 integration site. sgRNA sequences for insertion are shown in Table 1. The cells were cocultured with K562 cells for 48 hours and then the PrimeR induced CAR MFI was compared to the PrimeR MFI.

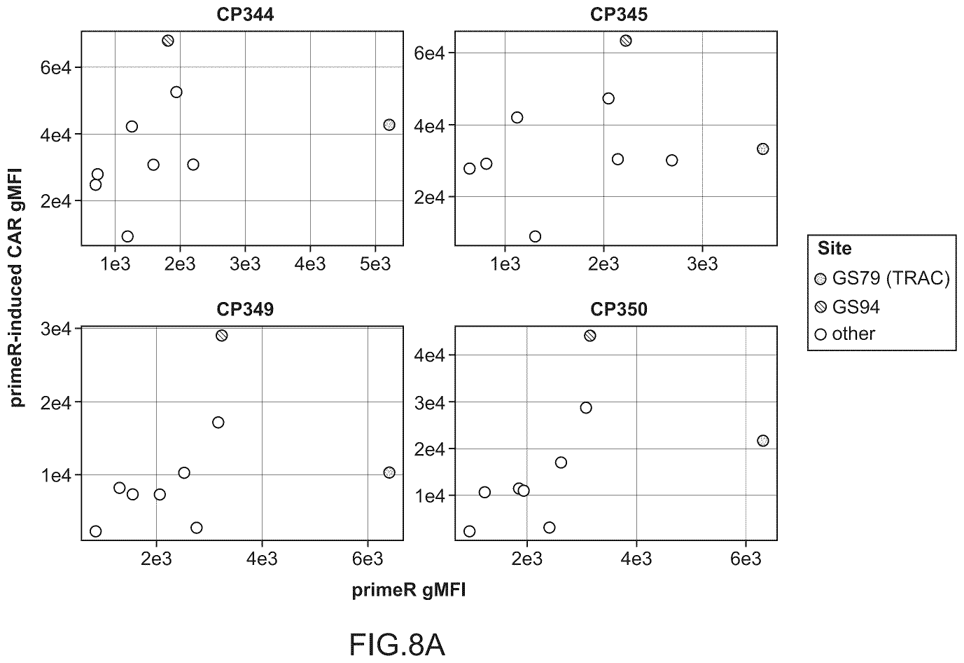

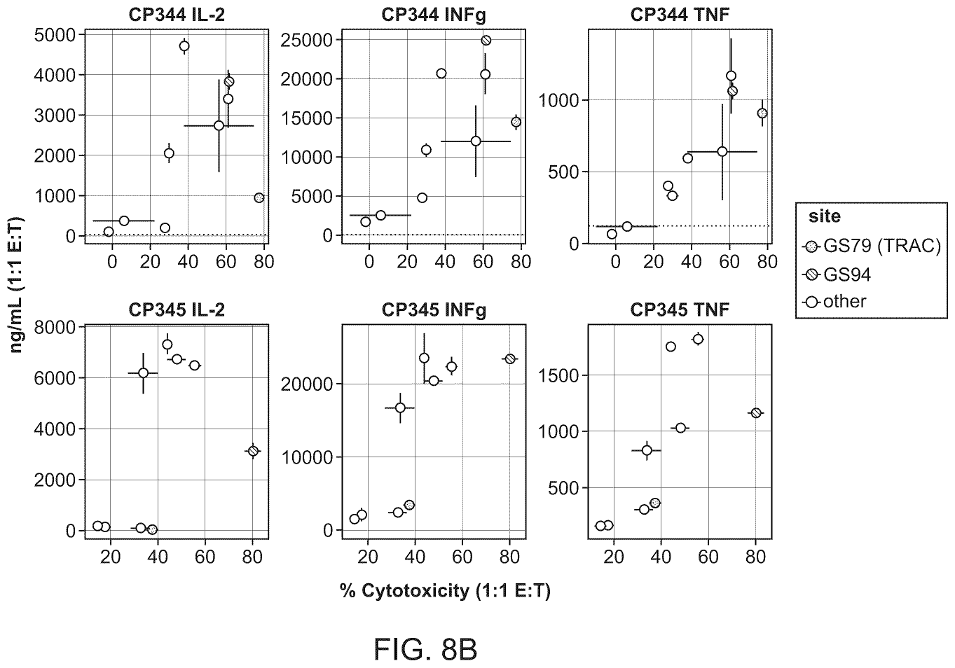

CD3-CD28 Dynabead-activated T cells were electroporated with sgRNA/Cas9 RNPs targeting GS94, as well as HDRTs with homology arms directing HDR-mediated integration into the indicated sites. The T cells were cocultured with K562 cells expressing the primeR target (K562 single expression cells) at day 7 post-electroporation. Cells were then stained with anti-FLAG antibody 48 h post initiation of coculture and analyzed on an Attune NxT flow cytometer. The results revealed that GS94 yields superior CAR induction with high prime R expression following the 48-hour coculture with the K562 cells. See FIG. 8A. GS94 resulted in prime antigen-dependent CAR expression that was approximately two-fold higher than the expression in several other candidate integration sites as well as the TRAC integration site. Additionally, on average, the prime receptor surface expression level was no less than 50% of expression level when using the TRAC integration site.

### Cytotoxicity and Cytokine Secretion

To evaluate the effect of the candidate integration site on cytotoxicity and cytokine secretion, T cells that had undergone circuit cassette integration with PrimeR at GS79 (TRAC) integration site and GS94 integration site were cocultured with K562 cells expressing the primeR target (K562 single expression cells) for 48 hours. The cells were treated at a 1:1 effector:target cell ratio (1:1 E:T)). Briefly, T cells generated as described above were cocultured with K562 cells dual expressing the primeR target and the CAR target (K562 dual expression cells) at day 7 post-electroporation. 48h post initiation of coculture, supernatants were collected and analyzed via Luminex for cytokine levels. The cytokines measure were IL-2, INFg, and TNF. Cytotoxicity was analyzed by measuring luciferase activity of remaining target cells after 48h. Each of the data points in FIG. 8B represent two replicates and the lines represent the range of cytotoxicity for the replicates. As shown in FIG. 8B, the GS94 integration sites resulted in superior cytotoxic ability and cytokine secretion following the 48-hour coculture with K562 dual expression cells.

### Prime-Independent Cytotoxicity

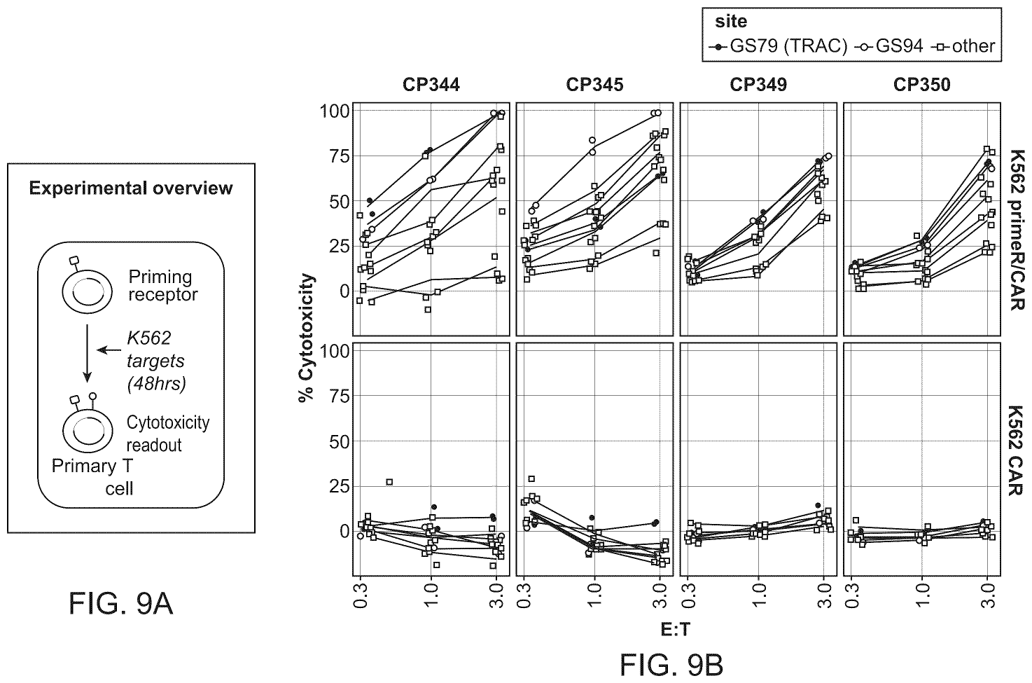

To compare the effect of the candidate integration site on cytotoxicity versus prime-independent cytotoxicity, T cells that had undergone circuit cassette integration with PrimeR at GS79 (TRAC) integration site and GS94 integration site were cocultured with K562 dual expression cells (“K562 primeR/CAR) or K562 cells only expressing the CAR target (K562 CAR) at day 7 post-electroporation for 48 hours. 0.3, 1.0, and 3.0 E:T cell ratios were tested. See FIG. 9A and FIG. 9B. At 48h post initiation of coculture, cytotoxicity was analyzed by measuring luciferase activity of remaining target cells As shown in FIG. 9B, the GS94 integration site resulted in equivalent cytotoxic potential to the TRAC integration site and there was no prime-independent cytotoxicity.

### Prime-Independent Cytokine Section

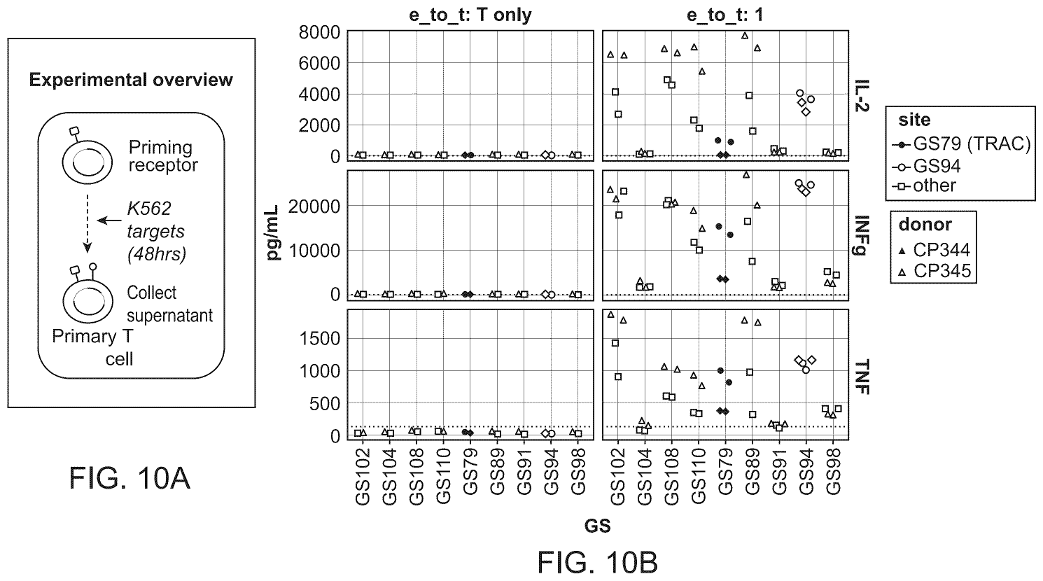

To compare the effect of the candidate integration site on cytotoxicity versus prime-independent cytotoxicity, T cells that had undergone circuit cassette integration with PrimeR at GS79 (TRAC) integration site and GS94 integration site, generated as described above, were cocultured with K562 dual expression cells. A group that had target cells only (E:T=0; Targets only) was compared to a group with an E:T cell ratio of 1. Following the 48 h coculture with the K562 dual expression cells, supernatant was collected and analyzed via Luminex for cytokine levels to measure secretion of IL-2, INFg and TNF cytokines. See FIG. 10A and FIG. 10B. As shown in FIG. 10B, the GS94 integration site resulted in equivalent cytokine secretion to the TRAC integration site and there was no prime-independent secretion of IL-2, INFg or TNF.

### Prime-Independent CAR Expression

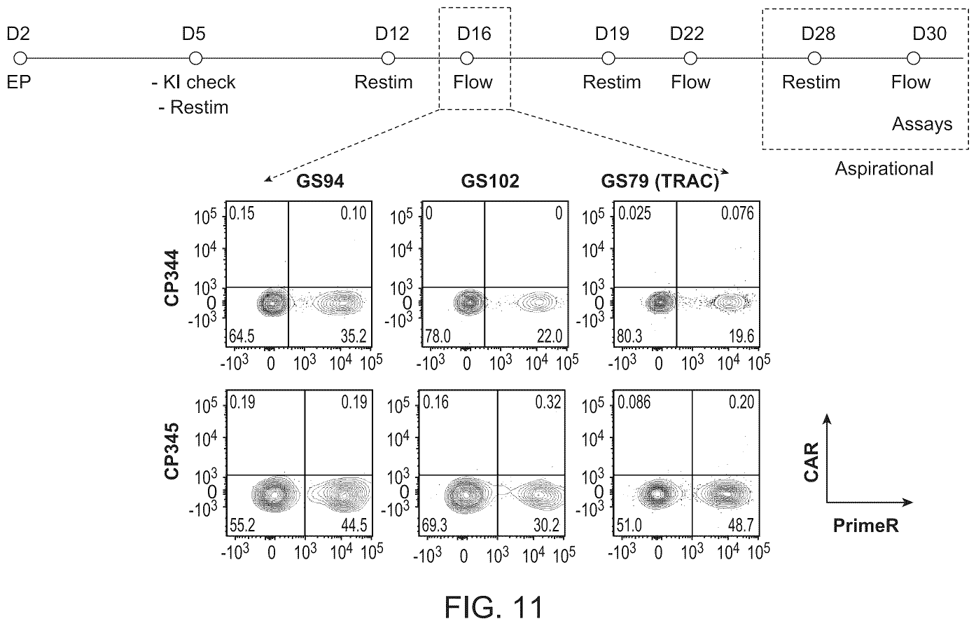

To evaluate the effect of the candidate integration site on prime-independent CAR expression, T cells that had undergone circuit cassette integration with PrimeR at GS79 (TRAC) integration site, and GS94 integration site, and GS102 integration site were cultured in vitro for 32 days. The cells were treated with repetitive CD3/CD28 stimulation at days 5, 12, 19 and 28 of the experiment. On Day 16, the cells were evaluated for CAR expression using a flow cytometry assay. As shown in FIG. 11, T cell activation through TCR did not result in primeR-independent CAR expression from circuit cassette integration at the candidate integration sites.

T cells generated as described above were cultured in 96-well plates, with T cell growth medium being exchanged every 2 days. At days 5, 12, 19 and 28, T cells were stimulated with 1:1 CD3/CD28 Dynabeads. Cells were analyzed for PrimeR expression by myc epitope tag staining, and for CAR expression by FLAG epitope tag staining at the indicated time points. Flow analysis was performed on an Attune NxT flow cytometer.

### Example 3: Evaluating Stability of Prime Receptor Expression from a Large Insert Over Several Weeks

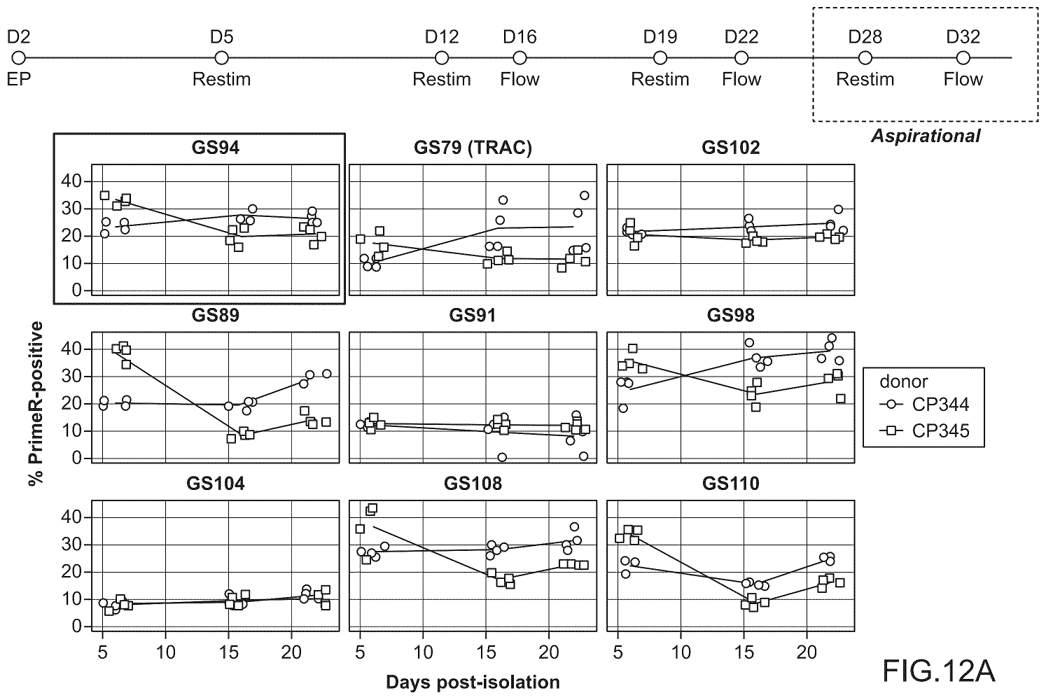

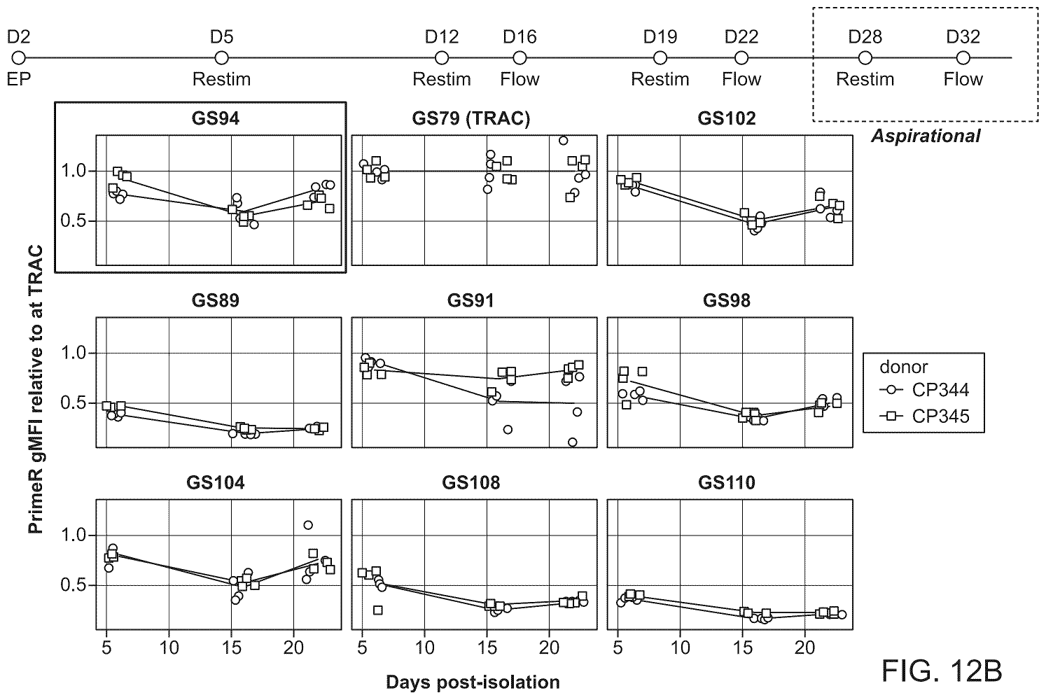

To evaluate the effect of the candidate integration sites on stable (sustained) expression of PrimeR, T cells that had undergone circuit cassette integration with PrimeR at the integration sites indicated in FIG. 12A were cultured in vitro for 32 days. Briefly, T cells generated as described above were cultured in 96-well plates, with T cell growth medium being exchanged every 2 days. At days 5, 12, 19 and 28, T cells were stimulated with 1:1 CD3/CD28 Dynabeads repetitive stimulation. Flow cytometry assays were run on days 16 and 32 using an Attune NxT flow cytometer. The cells were analyzed for PrimeR expression by myc epitope tag staining. As shown in FIGS. 12A and 12B, the GS94 integration site resulted in stable PrimeR expression over at least a 4-week period.

### Example 4: Evaluation of On-Target Editing Efficiency Using Non-Viral Insertion

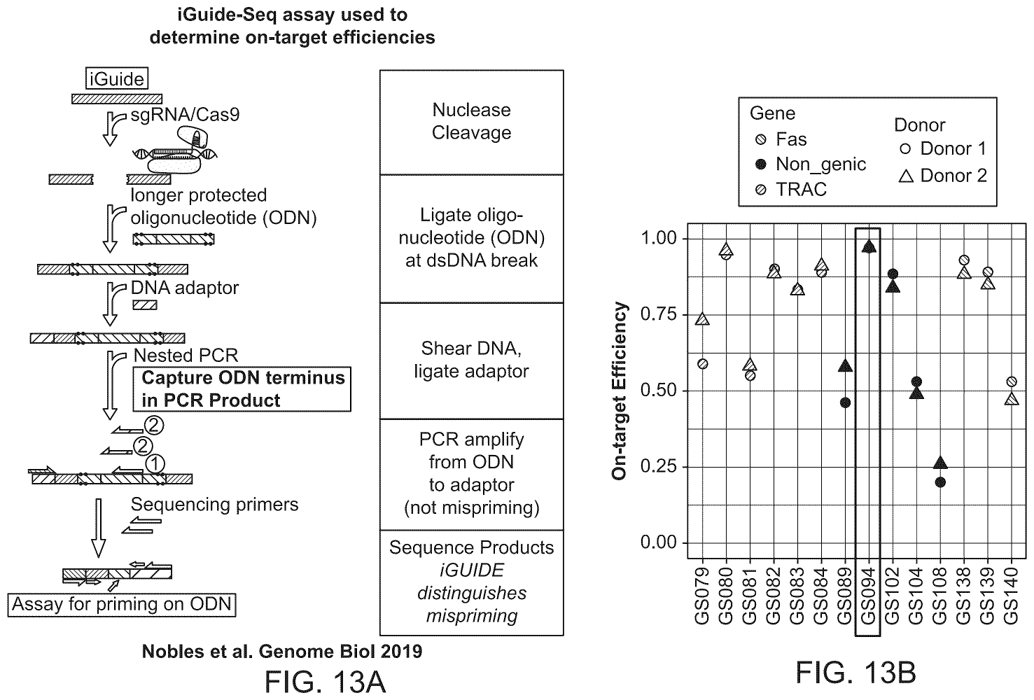

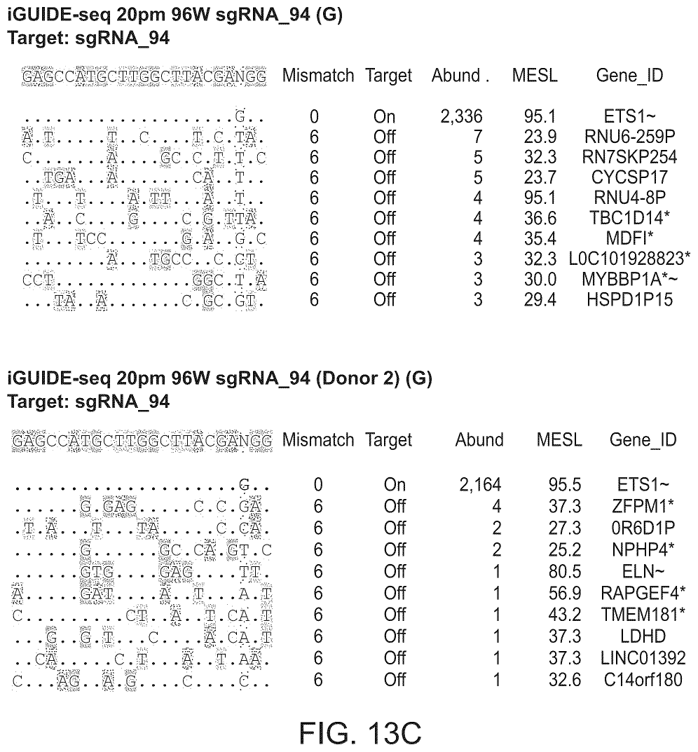

To evaluate the on-target editing efficiency of candidate knock-in sites, iGUIDE-Seq assay was used. The methods used for conducting the iGUIDE-Seq assay are illustrated in FIG. 13A and provided in Nobles et al., Genome Biology (2019), which is hereby incorporated by reference in its entitrety. As shown in FIG. 13B, the GS94 integration site had the highest on-target editing efficiency of the evaluated candidate integration sites. As shown in FIG. 13C, GS94 resulted in no putative off-target editing as observed with two donors.

### Example 5: Evaluation of Various GSH Non-Viral Knock-Ins with Large Inserts

### Methods

Elevation prediction: Computational predictions of potential off-target sites from (gs94) were performed using Elevation-search (algorithm described in Listgarten et al. 2018. Prediction of off-target activities for the end-to-end design of CRISPR guide RNAs. Nat Biomed Engr 2, 37-48; software obtained from https://github.com/Microsoft/Elevation). All sites identified by Elevation-search were subjected to analysis using rhAmp-seq.

rhAmpSeq: 49 candidate off-target sites for GS94 identified by iGUIDE or the Elevation prediction algorithm and the GS94 target site were characterized by rhAmpSeq (Integrated DNA Technologies, Inc.). This targeted amplification enables NGS-based quantification of the editing occurring at numerous sites simultaneously. Genomic DNA from T cells from at least 2 donors that had been treated singly with each of the following 7 guides: GS84, GS94, GS95, GS96, GS102, GS108, and GS138 (Table 1) was isolated with the GenFind V3 DNA purification system (Beckman Coulter). Two separate rhAmpSeq amplification pools were used to cover the 50 loci, the procedure was performed as recommended by Integrated DNA Technologies for each of the samples. The rhAmpSeq libraries were sequenced on a MiniSeq with a Mid Output Kit (300-cycles) (Illumina). The CRISPResso2 algorithm (https://github.com/pinellolab/CRISPResso2) was used to determine the percentage of insertions and deletions at each of the amplified loci. Statistical significance (FDR-adjusted p-value ≤0.001) using a chi-squared test was only observed at the GS94 site.

RNA-seq: To evaluate changes induced by GS94 integration at the transcriptional level, a primeR/CAR circuit was integrated at the GS79 (TRAC), GS94, and GS102 integration sites. On day 6 post-integration, 1e6 edited cells were sorted using a BD FACSAria based on transgene expression. RNA was isolated from sorted T cells with the RNeasy kit (Qiagen). Purified RNA was converted into an NGS library using the TruSeq RNA Library Prep Kit v2 (Illumina). Libraries were sequenced on either the NovaSeq 6000 or NextSeq 550 instruments (Illumina). The STAR 2.7.3a aligner (Dobin A. et al. Bioinformatics. 2013. 29:15-21) was used to align the RNA-seq data against the reference human GRCh38 transcriptome and to obtain gene-level read counts. edgeR (Robinson M D et al. Bioinformatics. 2010. 26: 139-140) was used to compute differential expression, combining data across both donors. The only genes within 300 Kb of the GS94 site, ETS1 and FLI1, were not differentially expressed in cells with integration at the GS94 integration site compared to cells with integration at any of the other two loci. At an FDR-adjusted p-value cutoff of 0.01, the number of differentially expressed genes was minimal (<100 genes genome-wide).

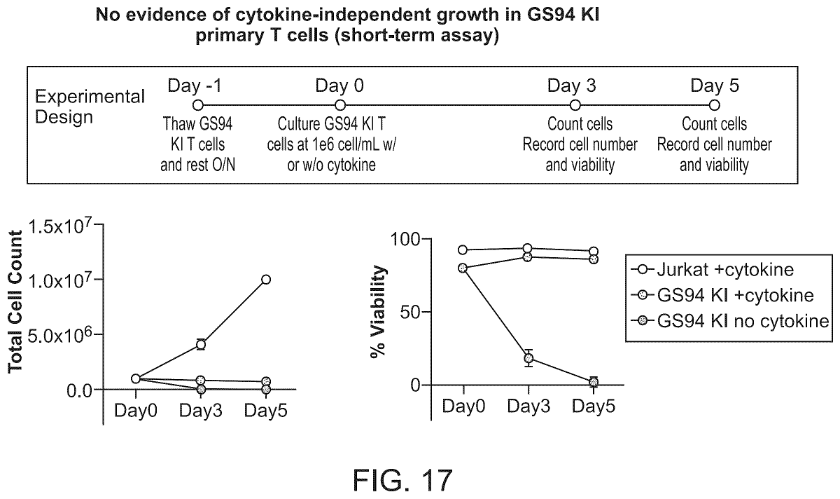

Cytokine-independent growth assay: To evaluate the safety of the primary T cells with GS94 locus KI, cytokine-independent growth assay was performed to evaluate the potential for oncogenic transformation. Briefly, primary human T cells that had undergone primeR/CAR circuit cassette integration at GS94 locus were thawed and recovered overnight. 1×10⁶ cells were then seeded in one well of a 24 well-GRex plate, culturing for 5 days in the medium with or without cytokines. Cell number and viability were recorded at days 0, 3 and 5. As a positive control, 1×10⁶ Jurkat cells were cultured in the medium without cytokines in parallel. As shown in FIG. 17, while GS94 KI T cells maintained good viability and total cell count when cultured with cytokine, the viability of GS94 KI T cells drastically decreased over the course of 5 days when cultured without cytokine and there was no viable cell left on day 5. The positive control Jurkat cells maintained good viability and expansion without cytokine throughout the assay. Taken together, this data shows that GS94 edited primary human T cells still depend on exogenous cytokine for growth, survival and expansion, therefore, there is no concern for cellular transformation.

### Results

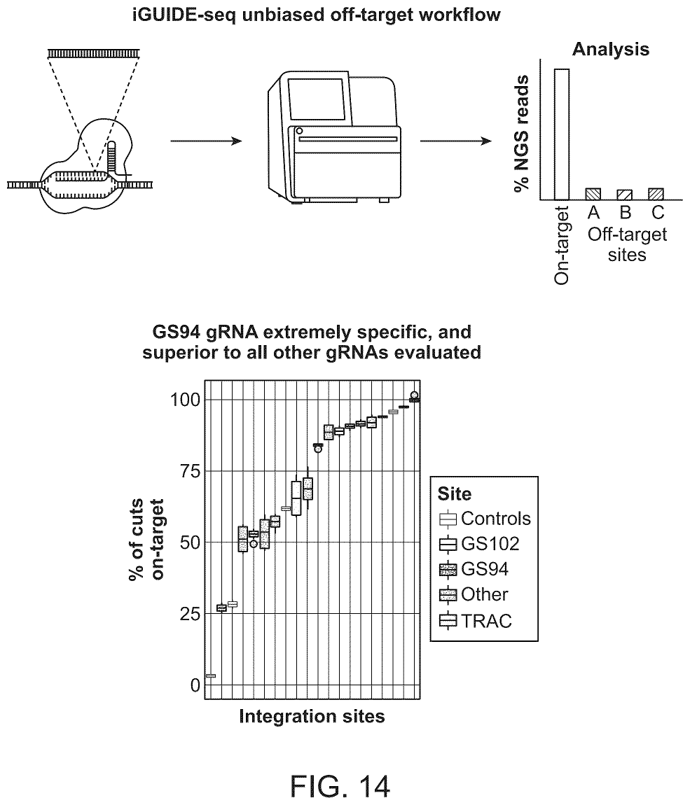

The specificity of CRISPR reagents (e.g. SpCas9 complexed with sgRNA) targeting candidate loci including GS94 was evaluated by iGUIDE-seq (FIG. 14). GS94-targeting CRISPR RNP showed the highest percentage of iGUIDE-seq oligo cassette trapping events of all candidates evaluated, and the control sgRNA sequences from the iGUIDE-seq paper showed similar specificities to what was reported in the original publication, suggesting that the assay performed as expected.

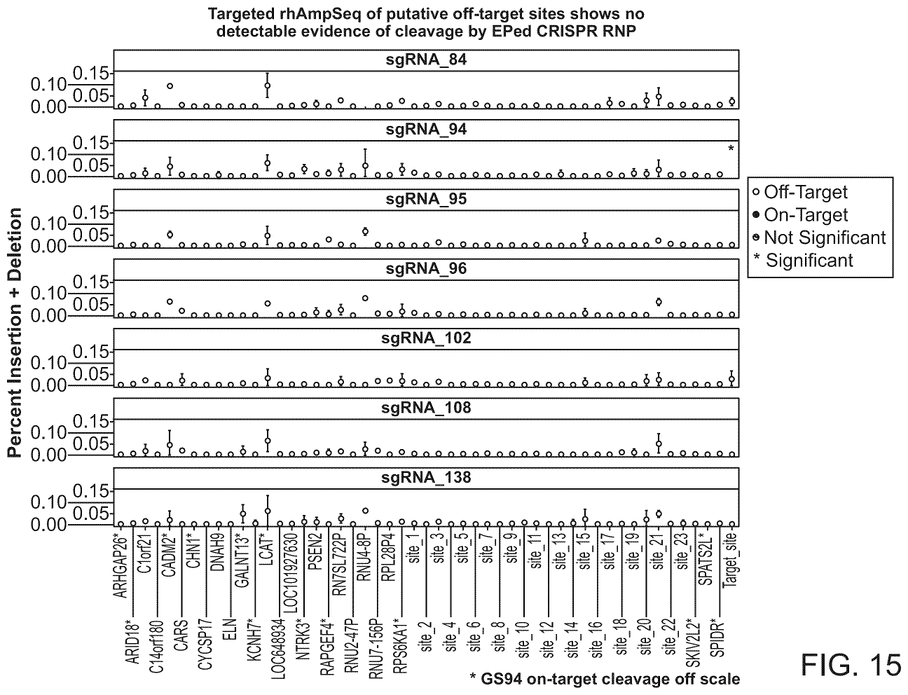

Putative off-target sites were taken from the iGUIDE-seq output, which already suggested that the putative sites were spurious. Additional target sites were predicted by a computational approach (Elevation software package). rhAmp-seq was used to prepare high-throughput sequencing libraries for each of the putative off-target sites, and the method was applied to DNA samples from T cells electroporated with CRISPR RNPs targeting the candidate target sites. The resulting NGS data were processed with CRISPResso2 software, and the frequency of insertions and deletions (indels) was taken as indication of CRISPR cleavage activity, as is common in the field. T cells electroporated with GS94-targeting CRISPR RNP showed no greater frequency of indels at the set of putative off-target sites than T cells treated with CRISPR RNP targeting other sites, consistent with the GS94-targeting CRISPR RNP having no consequential or detectable off-target activity, and therefore being the most specific out of the set evaluated (FIG. 15).

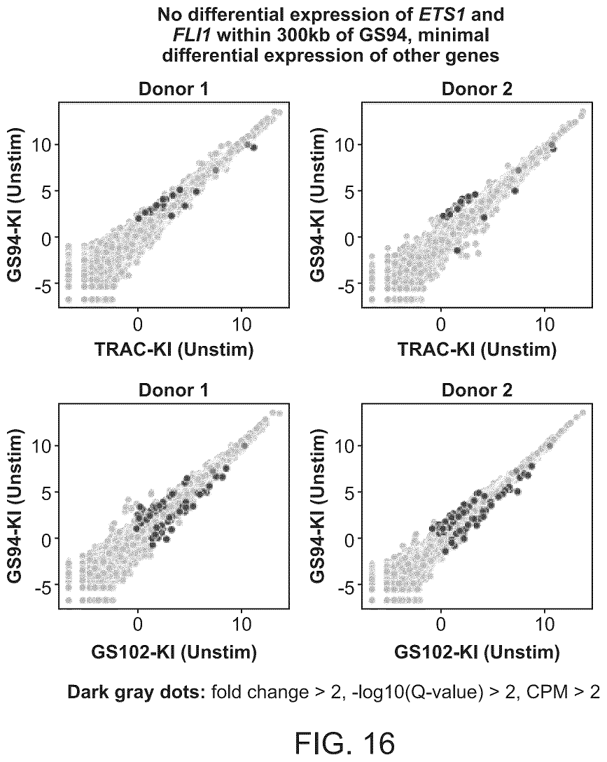

Potential effects of transgene integration at the GS94 site on the regulation of the T cell transcriptome were evaluated by knocking in a large cassette to the site, growing T cells for several days, sorting cells expressing the transgene within the cassette, and then collecting RNA from the cells. RNA-seq libraries were prepared and sequenced, and analysis of the resulting Illumina sequencing data revealed no biologically or statistically significant differences in expression of any genes within 300 kb of the GS94 site in cells with integrations at GS94 compared to cells with integration at TRAC or the GS102 sites (FIG. 16). Furthermore, other gene expression differences that reached statistical significance were minimal in number and in effect size, consistent with them being noise in the comparison.

To assess whether transgene integration at GS94 could confer a transformed phenotype, cells with integrations at the GS94 site were cultured with and without cytokines in vitro. Cells remained alive and viable with cytokine addition, but died without cytokine supplementation and lost their viability (FIG. 17). The positive control Jurkat cells remained viable and proliferated. Overall, this indicates that integration of a transgene at GS94 does not confer capacity for cytokine-independent growth, which is a hallmark of T cell transformation.

### Example 6: Evaluation of Non-Viral Insertion of a Large 8.3 kb Expression Cassette in GS94

Next, an 8.3 kb insert was inserted into a T cell at the GS94 safe harbor loci using materials/methods as previously described in Example 1. The increased length in the insert was due to additional protein coding sequences in the cassette. A diagram of the cassette is provided in FIG. 19B

Construct Generation

To generate plasmid constructs for knock-in, synthetic DNA was ordered from Twist, IDT and GENEWIZ and assembled via Gibson Assembly and Golden Gate Assembly. Plasmids contained homology arms homologous to sequences flanking the CRISPR target sites in the genome of 1.2 kb or 450 bp in length.

T Cell Engineering

T-cells were enriched from peripheral blood mononuclear cells (PBMCs) obtained from normal donor Leukopaks (STEMCELL Technologies) using Lymphoprep (STEMCELL Technologies) and the EasySep Human T-Cell Isolation Kit (STEMCELL Technologies). T-cells were subsequently activated with CD3/CD28 Dynabeads at 1:1 bead to cell ratio (ThermoFisher, 40203D) in TexMACS medium (Miltenyi 130-197-196) supplemented with 3% human AB serum (Gemini Bio) and 12.5 ng/ml human IL-7 and IL-15 (Miltenyi premium grade) and cultured at 37° C., 5% CO2 for 48 hours before electroporation.

CRISPR RNP were prepared by combining 120 μM sgRNA (Synthego) targeting DNA sequence GAGCCATGCTTGGCTTACGA (GS94, SEQ ID NO: 94), 62.5 μM sNLS-SpCas9-sNLS (Aldevron) and P3 buffer (Lonza) at a volume ratio of 5:1:3:6, and incubated for 15 minutes at room temperature. An optimized amount of plasmid DNA, determined by dose titration experiments (ranging from 0.5-3 micrograms) was mixed with 3.5 μl of RNP. T-cells were counted, debeaded, centrifuged at 90×G for 10 minutes and resuspended at 10\hat{ }6 cells/14.5 μl of P3 with supplement added (Lonza). 14.5 μl of T-cell suspension was added to the DNA/RNP mixture, transferred to Lonza 384-well nucleocuvette plate, and pulsed in a Lonza HT Nucleofector System with code EH-115. Cells were allowed to rest for 15 minutes at room temperature before transfer to 96-well plates (Sarstedt) in TexMACS medium supplemented with 12.5 ng/ml human IL-7 and IL-15 (Miltenyi premium grade).

Transgene expression was detected by staining with anti-Myc antibody (Cell Signaling Technology clone 9B11) and anti-Flag antibody (RnD systems, clone 1042E) and analyzed on an Attune NxT Flow Cytometer. Other antibodies used were live/dead Fixable Near-IR (Thermo Fisher), TCRalpha/beta antibody (BioLegend clone IP26), CD4 antibody (BioLegend clone RPA-T4), CD8 antibody (BioLegend clone SK1).

Priming Receptor Induction

To assess functional activity of transgene (ie. synthetic circuit), edited T cells were co-cultured with target cell line expressing priming antigen at 1:1 E:T ratio, and incubated for 24 hrs. T cells were harvested and stained with anti-Myc and anti-Flag antibodies to assess for Prime Receptor and CAR expression, respectively.

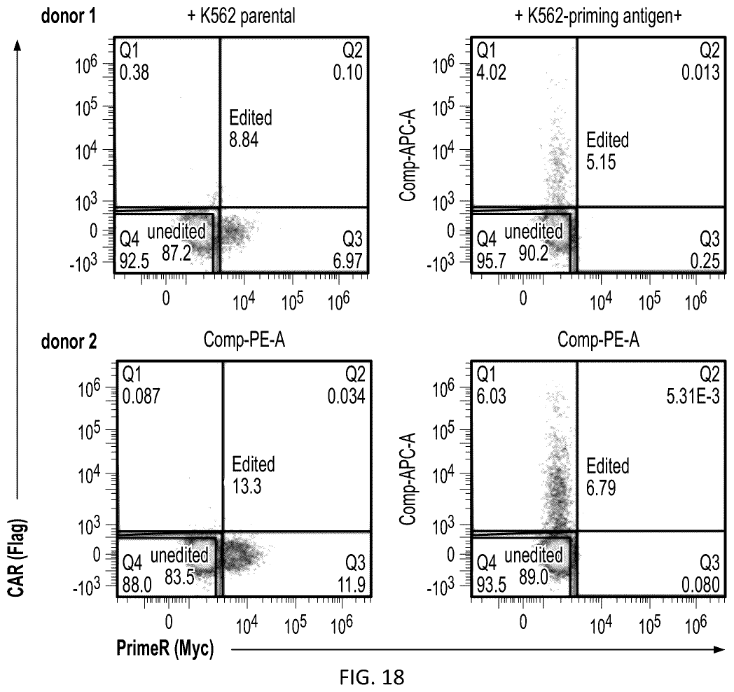

To assess whether the 8.3 kb transgene integration at GS94 resulted in functional knock-in, cells were cultured with parental K562 cells and K562 cells expressing the cognate priming antigen at a 1:1 E:T cell ratio. Cells were assayed by flow cytometry after 48 hours as previously described. K562 cells with priming antigen induced CAR expression, while control parental K562 cells did not (FIG. 18). Overall, this indicates that the PrimeR induced CAR expression after insertion of a 8.3 kb transgene circuit.

### Example 7: In Vivo Use of T Cells Comprising a CAR Expressing Cassette

In vivo efficacy of T cells with a transgene cassette expressing a CAR recognizing a tumor antigen, or a CAR recognizing a tumor antigen under control of a priming receptor recognizing an antigen in the anatomical vicinity of the tumor, is assessed against human tumor cells such as K562 engineered to express the CAR antigen or to express antigens recognized by both the priming receptor and the CAR. Tumor cells (e.g. 1e6) are subcutaneously injected into the flank of NSG mice (Jackson Laboratories). Tumor growth is assessed by dimensional measurement by calipers every 2-4 days. When the tumor volume reaches ˜100 cubic mm, mice are intravenously injected with 5e6 T cells with a CAR or primeR-CAR circuit cassette integrated at a specific site by CRISPR-mediated insertion, or with T cells engineered with CRISPR RNP alone, or with PBS alone as a sham injection. Tumor growth is monitored and mice are euthanized when tumor volume reaches 2000 cubic mm. Peripheral blood is bled from mice through a retro-orbital procedure, and flow cytometry and/or ddPCR is used to observe engineered T cell expansion over time. At time of sacrifice, spleen, blood, tumor and/or other tissue is analyzed via flow cytometry, ddPCR, and/or immunohistochemistry for the presence of engineered T cells. The results demonstrate that T cells engineered with cassette integration at one of the defined genomic loci lead to tumor regression and clearance in injected mice as compared to T cells without cassette integration, and that engineered T cells are detectable in the peripheral blood and tissues of injected mice.

TABLE 1
sgRNA sequences

| | | | | | Median (% |
|---|---|---|---|---|---|
| | | | | | Modified), |
| | | | | | summarized |
| | | | sgRNA | | from 2 |
| sgRNA | | sgRNA start | Target | Integration | donors, 2 |
| ID | sgRNA Sequence | coor GRCH38 | Loci | Site | primersets |
| sgRNA_ | GCACCTGAATACCACGCCT | chr16: 88811818 | APRT | APRT | 79.28 |
| 1 | G (SEQ ID NO: 1) | | | | |
| sgRNA_ | CGCCTGCGATGTAGTCGAT | chr16: 88811551 | APRT | APRT | 78.60 |
| 2 | G (SEQ ID NO: 2) | | | | |
| sgRNA_ | CAGGACGGGCGAGATGTCC | chr16: 88811640 | APRT | APRT | 85.25 |
| 3 | C (SEQ ID NO: 3) | | | | |
| sgRNA_ | CTGAATCTTTGGAGTACCT | chr15: 44715425 | B2M | B2M | 78.51 |
| 4 | G (SEQ ID NO: 4) | | | | |
| sgRNA_ | GGCCACGGAGCGAGACAT | chr15: 44711550 | B2M | B2M | 94.75 |
| 5 | CT (SEQ ID NO: 5) | | | | |
| sgRNA_ | AAGTCAACTTCAATGTCGG | chr15: 44715515 | B2M | B2M | 70.97 |
| 6 | A (SEQ ID NO: 6) | | | | |
| sgRNA_ | GCTTGGAGGCCTGATCAGC | chr19: 36141111 | CAPNS1 | CAPNS1 | 89.34 |
| 7 | G (SEQ ID NO: 7) | | | | |
| sgRNA_ | CTTATCTCTTCGCAGCGAG | chr19: 36142301 | CAPNS1 | CAPNS1 | 91.09 |
| 8 | G (SEQ ID NO: 8) | | | | |
| sgRNA_ | CACACATTACTCCAACATT | chr19: 36142676 | CAPNS1 | CAPNS1 | 71.98 |
| 9 | G (SEQ ID NO: 9) | | | | |
| sgRNA_ | TTCCGCAAAATAGAGCCCC | chr3: 105746019 | CBLB | CBLB | 91.55 |
| 10 | A (SEQ ID NO: 10) | | | | |
| sgRNA_ | TGCACAGAACTATCGTACC | chr3: 105751622 | CBLB | CBLB | 91.43 |
| 11 | A (SEQ ID NO: 11) | | | | |
| sgRNA_ | GCAATAAGACTCTTTAAAG | chr3: 105853470 | CBLB | CBLB | 76.18 |
| 12 | A (SEQ ID NO: 12) | | | | |
| sgRNA_ | CAAAGAGATTACGAATGCC | chr1: 116754658 | CD2 | CD2 | 89.80 |
| 13 | T (SEQ ID NO: 13) | | | | |
| sgRNA_ | CAAGGCACCCCAGGTTTCC | chr1: 116754663 | CD2 | CD2 | 92.70 |
| 14 | A (SEQ ID NO: 14) | | | | |
| sgRNA_ | TTACGAATGCCTTGGAAAC | chr1: 116754666 | CD2 | CD2 | 92.82 |
| 15 | C (SEQ ID NO: 15) | | | | |
| sgRNA_ | CAGAGACGCATCTGACCCT | chr11: 118315540 | CD3E | CD3E | 90.96 |
| 16 | C (SEQ ID NO: 16) | | | | |
| sgRNA_ | CATGCAGTTCTCACACACT | chr11: 118313715 | CD3E | CD3E | 87.47 |
| 17 | G (SEQ ID NO: 17) | | | | |
| sgRNA_ | GTGTGAGAACTGCATGGAG | chr11: 118313715 | CD3E | CD3E | 86.65 |
| 18 | A (SEQ ID NO: 18) | | | | |
| sgRNA_ | TCTCATTTCAGGAAACCAC | chr11: 118349748 | CD3G | CD3G | 87.24 |
| 19 | T (SEQ ID NO: 19) | | | | |
| sgRNA_ | AGTCATACACCTTAACCAA | chr11: 118349754 | CD3G | CD3G | 87.99 |
| 20 | G (SEQ ID NO: 20) | | | | |
| sgRNA_ | TTCAAGGAAACCAGTTGAG | chr11: 118352458 | CD3G | CD3G | 86.55 |
| 21 | G (SEQ ID NO: 21) | | | | |
| sgRNA_ | GAGCCTTGCCTGGAAATCT | chr11: 61118177 | CD5 | CD5 | 84.03 |
| 22 | G (SEQ ID NO: 22) | | | | |
| sgRNA_ | AAGCGTCAAAAGTCTGCCA | chr11: 61118324 | CD5 | CD5 | 89.19 |
| 23 | G (SEQ ID NO: 23) | | | | |
| sgRNA_ | CGTTCCAACTCGAAGTGCC | chr11: 61118121 | CD5 | CD5 | 83.11 |
| 24 | A (SEQ ID NO: 24) | | | | |
| sgRNA_ | GAGCGACTGGGACACGGT | chr9: 136866246 | EDF1 | EDF1 | 88.84 |
| 25 | GA (SEQ ID NO: 25) | | | | |
| sgRNA_ | GCTGCGCAAGAAGGGCCCT | chr9: 136866211 | EDF1 | EDF | 91.04 |
| 26 | A (SEQ ID NO: 26) | | | | |
| sgRNA_ | TTGTTCTGGCCAGCAGCCC | chr9: 136863433 | EDF1 | EDF1 | 85.98 |
| 27 | C (SEQ ID NO: 27) | | | | |
| sgRNA_ | CTTCCAGAGCCACATCATC | chr19: 48965791 | FTL | FTL | 93.10 |
| 28 | G (SEQ ID NO: 28) | | | | |
| sgRNA_ | GGGACTCACCAGAGAGAG | chr19: 48965601 | FTL | FTL | 88.86 |
| 29 | GT (SEQ ID NO: 29) | | | | |
| sgRNA_ | CGGTCGAAATAGAAGCCCT | chr19: 48965770 | FTL | FTL | 93.14 |
| 30 | A (SEQ ID NO: 30) | | | | |
| sgRNA_ | AAAAGGATATTGTGCAACT | chr10: 87933015 | PTEN | PTEN | 92.37 |
| 31 | G (SEQ ID NO: 31) | | | | |
| sgRNA_ | TGTGCATATTTATTACATC | chr10: 87933183 | PTEN | PTEN | 90.64 |
| 32 | G (SEQ ID NO: 32) | | | | |
| sgRNA_ | TTTGTGAAGATCTTGACCA | chr10: 87933087 | PTEN | PTEN | 85.36 |
| 33 | A (SEQ ID NO: 33) | | | | |
| sgRNA_ | TGTCATGCTGAACCGCATT | chr18: 12830972 | PTPN2 | PTPN2 | 87.94 |
| 34 | G (SEQ ID NO: 34) | | | | |
| sgRNA_ | CCACTCTATGAGGATAGTC | chr18: 12859219 | PTPN2 | PTPN2 | 92.45 |
| 35 | A (SEQ ID NO: 35) | | | | |
| sgRNA_ | TTGACATAGAAGAGGCACA | chr18: 12836828 | PTPN2 | PTPN2 | 93.96 |
| 36 | A (SEQ ID NO: 36) | | | | |
| sgRNA_ | GAGTACTACACTCAGCAGC | chr12: 6952098 | PTPN6 | PTPN6 | 89.61 |
| 37 | A (SEQ ID NO: 37) | | | | |
| sgRNA_ | TCACGCACAAGAAACGTCC | chr12: 6954872 | PTPN6 | PTPN6 | 82.74 |
| 38 | A (SEQ ID NO: 38) | | | | |
| sgRNA_ | AGGTCTCGGTGAAACCACC | chr12: 6951610 | PTPN6 | PTPN6 | 91.27 |
| 39 | T (SEQ ID NO: 39) | | | | |
| sgRNA_ | AGCATTATCCAAAGAGTCC | chr1: 198696873 | PTPRC | PTPRC | 88.88 |
| 40 | G (SEQ ID NO: 40) | | | | |
| sgRNA_ | ATATTAATTCTTACCAGTG | chr1: 198692370 | PTPRC | PTPRC | 88.95 |
| 41 | G (SEQ ID NO: 41) | | | | |
| sgRNA_ | AGCTTTAAATCAAGGTTCA | chr1: 198756176 | PTPRC | PTPRC | 96.89 |
| 42 | T (SEQ ID NO: 42) | | | | |
| sgRNA_ | ATCCCGAGCCCTAAGGTGC | chr11: 67436325 | PTPRCAP | PTPRCAP | 84.08 |
| 43 | A (SEQ ID NO: 43) | | | | |
| sgRNA_ | GGCAGCGCGGAGGACAGC | chr11: 67436285 | PTPRCAP | PTPRCAP | 97.74 |
| 44 | GT (SEQ ID NO: 44) | | | | |
| sgRNA_ | CTCAGGGGGCTACTACCAC | chr11: 67436170 | PTPRCAP | PTPRCAP | 91.50 |
| 45 | C (SEQ ID NO: 45) | | | | |
| sgRNA_ | GTCACCGACGAGACCAGA | chr5: 82277810 | RPS23 | RPS23 | 79.40 |
| 46 | AG (SEQ ID NO: 46) | | | | |
| sgRNA_ | GTCGTGGACTTCGTACTGC | chr5: 82277843 | RPS23 | RPS23 | 83.07 |
| 47 | T (SEQ ID NO: 47) | | | | |
| sgRNA_ | TAATTTTTAGGCAAGTGTC | chr5: 82277860 | RPS23 | RPS23 | 61.94 |
| 48 | G (SEQ ID NO: 48) | | | | |
| sgRNA_ | TTAGCTGTTAGACTTGAAT | chr14: 51993810 | RTRAF | RTRAF | 85.50 |
| 49 | A (SEQ ID NO: 49) | | | | |
| sgRNA_ | CGAGAGCCGTCAACTTGCG | chr14: 51989652 | RTRAF | RTRAF | 85.64 |
| 50 | T (SEQ ID NO: 50) | | | | |
| sgRNA_ | CGGCTTCAACTGCAAAGGT | chr14: 51989700 | RTRAF | RTRAF | 88.77 |
| 51 | G (SEQ ID NO: 51) | | | | |
| sgRNA_ | TATGAAAAAGCAGAGCGA | chr15: 43793025 | SERF2 | SERF2 | 89.61 |
| 52 | CT (SEQ ID NO: 52) | | | | |
| sgRNA_ | TCTGGCGGGCGAGCTCACG | chr15: 43792989 | SERF2 | SERF2 | 86.73 |
| 53 | C (SEQ ID NO: 53) | | | | |
| sgRNA_ | CTCACGCTGGTTACCGCCT | chr15: 43792977 | SERF2 | SERF2 | 80.57 |
| 54 | A (SEQ ID NO: 54) | | | | |
| sgRNA_ | AAAGATTACGAACTTCCCT | chr12: 46207559 | SLC38A1 | SLC38A1 | 92.24 |
| 55 | G (SEQ ID NO: 55) | | | | |
| sgRNA_ | GTTAAAAACAGACATGCCT | chr12: 46229232 | SLC38A1 | SLC38A1 | 91.51 |
| 56 | A (SEQ ID NO: 56) | | | | |
| sgRNA_ | ATGCCTAAGGAGGTTGTAC | chr12: 46229246 | SLC38A1 | SLC38A1 | 79.48 |
| 57 | C (SEQ ID NO: 57) | | | | |
| sgRNA_ | CTCCAGGTATCCCATCGAA | chr18: 47869418 | SMAD2 | SMAD2 | 79.53 |
| 58 | A (SEQ ID NO: 58) | | | | |
| sgRNA_ | CACCAAATACGATAGATCA | chr18: 47870532 | SMAD2 | SMAD2 | 86.61 |
| 59 | G (SEQ ID NO: 59) | | | | |
| sgRNA_ | TGGCGGCGTGAATGGCAAG | chr18: 47896729 | SMAD2 | SMAD2 | 82.91 |
| 60 | A (SEQ ID NO: 60) | | | | |
| sgRNA_ | TAGGATGGTAGCACACAAC | chr16: 11255478 | SOCS1 | SOCS1 | 92.25 |
| 61 | C (SEQ ID NO: 61) | | | | |
| sgRNA_ | CAGCAGCAGAGCCCCGAC | chr16: 11255432 | SOCS1 | SOCS1 | 83.79 |
| 62 | GG (SEQ ID NO: 62) | | | | |
| sgRNA_ | CGGCGTGCGAACGGAATGT | chr16: 11255296 | SOCS1 | SOCS1 | 84.24 |
| 63 | G (SEQ ID NO: 63) | | | | |
| sgRNA_ | TATAGACGCTGCCCGACGT | chr15: 40038895 | SRP14 | SRP14 | 95.12 |
| 64 | C (SEQ ID NO: 64) | | | | |
| sgRNA_ | TCCAAAGAAGGGTACTGTG | chr15: 40038368 | SRP14 | SRP14 | 92.14 |
| 65 | G (SEQ ID NO: 65) | | | | |
| sgRNA_ | ACAGTACCCTTCTTTGGAA | chr15: 40038358 | SRP14 | SRP14 | 65.82 |
| 66 | T (SEQ ID NO: 66) | | | | |
| sgRNA_ | GCGACGGGCGCATCTACGT | chr12: 120469572 | SRSF9 | SRSF9 | 83.68 |
| 67 | G (SEQ ID NO: 67) | | | | |
| sgRNA_ | CCCGACCTCCATAAGTCCT | chr12: 120465700 | SRSF9 | SRSF9 | 92.56 |
| 68 | G (SEQ ID NO: 68) | | | | |
| sgRNA_ | GGGGTCCTCGAAGCGCACG | chr12: 120469426 | SRSF9 | SRSF9 | 89.94 |
| 69 | A (SEQ ID NO: 69) | | | | |
| sgRNA_ | TGCTCTGTTTAGAAGATGA | chr5: 32591641 | SUB1 | SUB1 | 79.36 |
| 70 | C (SEQ ID NO: 70) | | | | |
| sgRNA_ | ATATTCTTTTCTAGTTAAAG | chr5: 32591566 | SUB1 | SUB1 | 70.93 |
| 71 | (SEQ ID NO: 71) | | | | |
| sgRNA_ | CCTGTAAAGAAACAAAAG | chr5: 32591614 | SUB1 | SUB1 | 93.66 |
| 72 | AC (SEQ ID NO: 72) | | | | |
| sgRNA_ | TGGAGAAAGACGTAACTTC | chr4: 105234315 | TET2 | TET2 | 83.53 |
| 73 | G (SEQ ID NO: 73) | | | | |
| sgRNA_ | TCTGCCCTGAGGTATGCGA | chr4: 105234747 | TET2 | TET2 | 90.97 |
| 74 | T (SEQ ID NO: 74) | | | | |
| sgRNA_ | ATTCCGCTTGGTGAAAACG | chr4: 105235656 | TET2 | TET2 | 89.62 |
| 75 | A (SEQ ID NO: 75) | | | | |
| sgRNA_ | CAGGCACAATAGAAACAA | chr3: 114295571 | TIGIT | TIGIT | 92.65 |
| 76 | CG (SEQ ID NO: 76) | | | | |
| sgRNA_ | CCATTTGTAATGCTGACTT | chr3: 114295700 | TIGIT | TIGIT | 60.75 |
| 77 | G (SEQ ID NO: 77) | | | | |
| sgRNA_ | CTGGGTCACTTGTGCCGTG | chr3: 114295634 | TIGIT | TIGIT | 87.99 |
| 78 | G (SEQ ID NO: 78) | | | | |
| sgRNA_ | GTCAGGGTTCTGGATATCT | chr14: 22547508 | TRAC | TRAC | 98.20 |
| 79 | G (SEQ ID NO: 79) | | | | |
| sgRNA_ | TGGATTTAGAGTCTCTCAG | chr14: 22547541 | TRAC | TRAC | 88.15 |
| 80 | C (SEQ ID NO: 80) | | | | |
| sgRNA_ | CTGCGGCTGTGGTCCAGCT | chr14: 22550661 | TRAC | TRAC | 94.77 |
| 81 | G (SEQ ID NO: 81) | | | | |
| sgRNA_ | ACAAAACTGTGCTAGACAT | chr14: 22547658 | TRAC | TRAC | 87.86 |
| 82 | G (SEQ ID NO: 82) | | | | |
| sgRNA_ | TTCTTCCCCAGCCCAGGTA | chr14: 22547778 | TRAC | TRAC | 89.85 |
| 83 | A (SEQ ID NO: 83) | | | | |
| sgRNA_ | CGTCATGAGCAGATTAAAC | chr14: 22550625 | TRAC | TRAC | 95.81 |
| 84 | C (SEQ ID NO: 84) | | | | |
| sgRNA_ | GAGAGCGCCTGCGACCCGA | chr19: 58544980 | TRIM28 | TRIM28 | 89.44 |
| 85 | G (SEQ ID NO: 85) | | | | |
| sgRNA_ | CCAGCGGGTGAAGTACACC | chr19: 58544869 | TRIM28 | TRIM28 | 94.79 |
| 86 | A (SEQ ID NO: 86) | | | | |
| sgRNA_ | GGAGCGCTTTTCGCCGCCA | chr19: 58544839 | TRIM28 | TRIM28 | 91.81 |
| 87 | G (SEQ ID NO: 87) | | | | |
| sgRNA_ | TGAGGCCTGGACCTTATGC | chr10: 33134193 | chr10: 33130000- | desert_1 | 69.44 |
| 88 | A (SEQ ID NO: 88) | | 33140000 | (GS88) | |
| sgRNA_ | CCTGGTGGAGTGAACCATG | chr10: 33132917 | chr10: 33130000- | desert_1 | 95.25 |
| 89 | A (SEQ ID NO: 89) | | 33140000 | (GS89) | |
| sgRNA_ | CAAGCACTTAGGTTCCCCT | chr10: 33134633 | chr10: 33130000- | desert_1 | 91.13 |
| 90 | G (SEQ ID NO: 90) | | 33140000 | (GS90) | |
| sgRNA_ | GGTCTCCCTACAATTCAGC | chr10: 72294568 | chr10: 72290000- | desert_2 | 92.02 |
| 91 | G (SEQ ID NO: 91) | | 72300000 | (GS91) | |
| sgRNA_ | CACAGCGCGTGACTGCAAT | chr10: 72298268 | chr10: 72290000- | desert_2 | 90.22 |
| 92 | G (SEQ ID NO: 92) | | 72300000 | (GS92) | |
| sgRNA_ | TCTGGGGCACCAATTCTAG | chr10: 72292786 | chr10: 72290000- | desert_2 | 86.35 |
| 93 | G (SEQ ID NO: 93) | | 72300000 | (GS93) | |
| sgRNA_ | GAGCCATGCTTGGCTTACG | chr11: 128342576 | chr11: 128340000- | desert_3 | 91.24 |
| 94 | A (SEQ ID NO: 94) | | 128350000 | (GS94) | |
| sgRNA_ | GTACAAGTACTTATCTCAT | chr11: 128343592 | chr11: 128340000- | desert_3 | 89.02 |
| 95 | G (SEQ ID NO: 95) | | 128350000 | (GS95) | |
| sgRNA_ | GAGATAACAACATAACAA | chr11: 128347170 | chr11: 128340000- | desert_3 | 96.47 |
| 96 | CA (SEQ ID NO: 96) | | 128350000 | (GS96) | |
| sgRNA_ | CATATTCCATAGTCTTTGG | chr11: 65425000 | chr11: 65425000- | desert_4 | 88.54 |
| 97 | G (SEQ ID NO: 97) | | 65427000 | (GS97) | |
| | | | (NEAT1) | | |
| sgRNA_ | CTGCCCCTTAGCAACTTAG | chr11: 65425507 | chr11: 65425000- | desert_4 | 92.76 |
| 98 | G (SEQ ID NO: 98) | | 65427000 | (GS98) | |
| | | | (NEAT1) | | |
| sgRNA_ | TGTTTAAAAATATGTTGAC | chr11: 65426264 | chr11: 65425000- | desert_4 | 90.76 |
| 99 | A (SEQ ID NO: 99) | | 65427000 | (GS99) | |
| | | | (NEAT1) | | |
| sgRNA_ | CCAGGAATGGAAACTCACG | chr15: 92830315 | chr15: 92830000- | desert_5 | 87.84 |
| 100 | C (SEQ ID NO: 100) | | 92840000 | (GS100) | |
| sgRNA_ | GAGGCCGCTGAATTAACCC | chr15: 92831850 | chr15: 92830000- | desert_5 | 85.32 |
| 101 | G (SEQ ID NO: 101) | | 92840000 | (GS101) | |
| sgRNA_ | ATACACGCACACTTGCAGA | chr15: 92831131 | chr15: 92830000- | desert_5 | 99.92 |
| 102 | A (SEQ ID NO: 102) | | 92840000 | (GS102) | |
| sgRNA_ | GAGCAGACAGAAACCCAG | chr16: 11225670 | chr16: 11220000- | desert_6 | 87.92 |
| 103 | GG (SEQ ID NO: 103) | | 11230000 | (GS103) | |
| sgRNA_ | TGAGTCTCCAAACAGAACA | chr16: 11226284 | chr16: 11220000- | desert_6 | 88.53 |
| 104 | G (SEQ ID NO: 104) | | 11230000 | (GS104) | |
| sgRNA_ | TAATATCACTGACTTCACG | chr16: 11225029 | chr16: 11220000- | desert_6 | 87.65 |
| 105 | G (SEQ ID NO: 105) | | 11230000 | (GS105) | |
| sgRNA_ | TACACACAATGTAAGCAGC | chr2: 87467461 | chr2: 87460000- | desert_7 | 71.79 |
| 106 | A (SEQ ID NO: 106) | | 87470000 | (GS106) | |
| sgRNA_ | GGGAGCTCAATTCGAAACC | chr2: 87468809 | chr2: 87460000- | desert_7 | 65.89 |
| 107 | A (SEQ ID NO: 107) | | 87470000 | (GS107) | |
| sgRNA_ | TTGGACAGGTGAGACAGTC | chr2: 87467001 | chr2: 87460000- | desert_7 | 72.64 |
| 108 | G (SEQ ID NO: 108) | | 87470000 | (GS108) | |
| sgRNA_ | AAGCTCACTCAGATAGTGT | chr3: 186511316 | chr3: 186510000- | desert_8 | 76.89 |
| 109 | G (SEQ ID NO: 109) | | 186520000 | (GS109) | |
| sgRNA_ | CAGGAGAACCACCTTACAC | chr3: 186515260 | chr3: 186510000- | desert_8 | 86.31 |
| 110 | G (SEQ ID NO: 110) | | 186520000 | (GS110) | |
| sgRNA_ | GGACAGACCCTGATTCACA | chr3: 186519655 | chr3: 186510000- | desert_8 | 85.47 |
| 111 | A (SEQ ID NO: 111) | | 186520000 | (GS111) | |
| sgRNA_ | ACATGGCAGTCTATGAACA | chr3: 59451154 | chr3: 59450000- | desert_9 | 87.77 |
| 112 | G (SEQ ID NO: 112) | | 59460000 | (GS112) | |
| sgRNA_ | CCTATAGAGAGTACTACTT | chr3: 59456416 | chr3: 59450000- | desert_9 | 79.33 |
| 113 | G (SEQ ID NO: 113) | | 59460000 | (GS113) | |
| sgRNA_ | CCAACCGGGTCTTCATTAC | chr3: 59457029 | chr3: 59450000- | desert_9 | 92.21 |
| 114 | G (SEQ ID NO: 114) | | 59460000 | (GS114) | |
| sgRNA_ | TCAAGCGTAGAGTTCCGAG | chr8: 127993006 | chr8: 127980000- | desert_10 | 93.07 |
| 115 | T (SEQ ID NO: 115) | | 128000000 | (GS115) | |
| sgRNA_ | TCATGCAATTATGGACCCA | chr8: 127994663 | chr8: 127980000- | desert_10 | 89.40 |
| 116 | G (SEQ ID NO: 116) | | 128000000 | (GS116) | |
| sgRNA_ | CGGGAAAGTGACTGGCCAT | chr8: 127996766 | chr8: 127980000- | desert_10 | 87.45 |
| 117 | G (SEQ ID NO: 117) | | 128000000 | (GS117) | |
| sgRNA_ | TGAGATTGAAATCAAATCG | chr9: 7974159 | chr9: 7970000- | desert_11 | 84.84 |
| 118 | G (SEQ ID NO: 118) | | 7980000 | (GS118) | |
| sgRNA_ | TATGCAATATTCATCACGC | chr9: 7977914 | chr9: 7970000- | desert_11 | 85.44 |
| 119 | G (SEQ ID NO: 119) | | 7980000 | (GS119) | |
| sgRNA_ | AATGTGTTAAATCAAATGC | chr9: 7976895 | chr9: 7970000- | desert_11 | 83.48 |
| 120 | A (SEQ ID NO: 120) | | 7980000 | (GS120) | |

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A primary immune cell comprising at least one DNA template non-virally inserted into a target region of the genome of the cell, wherein the size of the DNA template is greater than or equal to about 5 kilobase pairs (kb).

2. The cell of claim 1, wherein the primary immune cell does not comprise a viral vector for introducing the DNA template into the primary immune cell.

3. The cell of claim 1, wherein the size of the DNA template is greater than or equal to about 8.0 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10.0 kb or any size of DNA template in between these sizes.

4. (canceled)

5. (canceled)

6. (canceled)

7. (canceled)

8. The cell of claim 1, wherein the target region of the genome of the cell is a T Cell Receptor Alpha Constant (TRAC) locus or a genomic safe harbor (GSH).

9. The cell of claim 1, wherein the DNA template comprises a heterologous sequence.

10. (canceled)

11. The cell of claim 1, wherein the DNA template comprises a priming receptor comprising a transcription factor, a chimeric antigen receptor (CAR), or a chimeric antigen receptor (CAR) and a priming receptor comprising a transcription factor.

12.-22. (canceled)

23. The cell of claim 11, wherein the priming receptor comprises, in an N terminus to C terminus direction:
a. an extracellular antigen-binding domain having a binding affinity for an antigen;
b. a transmembrane domain comprising one or more ligand-inducible proteolytic cleavage sites; and
c. an intracellular domain comprising a human or humanized transcriptional effector, wherein binding of an antigen to the extracellular antigen-binding domain results in cleavage at the ligand-inducible proteolytic cleavage site thereby releasing the intracellular domain.

24. (canceled)

25. (canceled)

26. The cell of claim 11, wherein the CAR comprises,
from N-terminus to C-terminus,
a. an extracellular antigen-binding domain having a binding affinity for an antigen;
b. a transmembrane domain;
c. an intracellular co-stimulatory domain; and
d. an intracellular activation domain.

27. (canceled)

28. (canceled)

29. The cell of claim 1, wherein the immune cell is a primary human immune cell, optionally wherein the primary immune cell is a natural killer (NK) cell, a T cell, a CD8+ T cell, a CD4+ T cell, a primary T cell, or a T cell progenitor.

30.-33. (canceled)

34. The cell of claim 1, wherein the primary immune cell is virus-free.

35. A population of cells comprising a plurality of the primary immune cell of claim 1.

36. A primary immune cell comprising at least one DNA template inserted into a target region of the genome of the primary immune cell, wherein the size of the DNA template is greater than or equal to 5 kilobase pairs, and wherein the primary immune cell does not comprise a viral vector for introducing the DNA template into the primary immune cell, optionally wherein the at least one DNA template comprises a chimeric antigen receptor (CAR) and a priming receptor comprising a transcription factor.

37.-39. (canceled)

40. A method of treating a disease in a subject comprising administering the primary immune cell of claim 1 to the subject, optionally wherein the disease is cancer.

41. (canceled)

42. A non-viral vector comprising a DNA template, wherein the DNA template is greater than or equal to 5 kilobase nucleotides in size, and wherein the 5′ and 3′ ends of the DNA template comprise nucleotide sequences that are homologous to genomic sequences flanking an insertion site in a genome of a primary cell.

43. The non-viral vector of claim 42, wherein the size of the DNA template is greater than or equal to about 8.0 kb 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10.0 kb or any size of DNA template in between these sizes.

44.-64. (canceled)

65. A method of editing a primary immune cell, comprising:
a. providing a ribonucleoprotein complex (RNP)-DNA template complex, wherein
the RNP comprises a nuclease domain and a guide RNA, wherein the size of the DNA template is greater than or equal to 5 kilobase nucleotides in size, and wherein the 5′ and 3′ ends of the DNA template comprise nucleotide sequences that are homologous to genomic sequences flanking an insertion site in the genome of the primary immune cell;
b. non-virally introducing the RNP-DNA template complex into the primary immune cell, wherein the guide RNA specifically hybridizes to a target region of the genome of the primary immune cell, and wherein the nuclease domain cleaves the target region to create the insertion site in the genome of the primary immune cell; and
c. editing the primary immune cell via insertion of the DNA template into the insertion site in the genome of the primary immune cell.

66. The method of claim 65, wherein non-virally introducing comprises electroporation.

67. The method of claim 65, wherein the nuclease domain comprises a CRISPR-associated endonuclease (Cas), optionally a Cas9 nuclease.

68. The method of claim 65, wherein the size of the DNA template is greater than or equal to about 8.0 kb 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10.0 kb or any size of DNA template in between these sizes.

69.-100. (canceled)


SEQUENCE LISTING (146 sequences)

SEQ ID NO: 1 - DNA, 20 bp, synthetic construct
  misc_feature 1..20: note = Description of Artificial Sequence: Synthetic oligonucleotide
  source 1..20: mol_type = other DNA
gcacctgaataccacgcctg

SEQ ID NO: 2 - DNA, 20 bp, synthetic construct
  misc_feature 1..20: note = Description of Artificial Sequence: Synthetic oligonucleotide
  source 1..20: mol_type = other DNA
cgcctgcgatgtagtcgatg

SEQ ID NO: 3 - DNA, 20 bp, synthetic construct
  misc_feature 1..20: note = Description of Artificial Sequence: Synthetic oligonucleotide
  source 1..20: mol_type = other DNA
caggacgggcgagatgtccc

SEQ ID NO: 4 - DNA, 20 bp, synthetic construct
  misc_feature 1..20: note = Description of Artificial Sequence: Synthetic oligonucleotide
  source 1..20: mol_type = other DNA
ctgaatctttggagtacctg

SEQ ID NO: 5 - DNA, 20 bp, synthetic construct
  misc_feature 1..20: note = Description of Artificial Sequence: Synthetic oligonucleotide
  source 1..20: mol_type = other DNA
ggccacggagcgagacatct

SEQ ID NO: 6 - DNA, 20 bp, synthetic construct
  misc_feature 1..20: note = Description of Artificial Sequence: Synthetic oligonucleotide
  source 1..20: mol_type = other DNA
aagtcaacttcaatgtcgga

SEQ ID NO: 7 - DNA, 20 bp, synthetic construct
  misc_feature 1..20: note = Description of Artificial Sequence: Synthetic oligonucleotide
  source 1..20: mol_type = other DNA
gcttggaggcctgatcagcg

SEQ ID NO: 8 - DNA, 20 bp, synthetic construct
  misc_feature 1..20: note = Description of Artificial Sequence: Synthetic oligonucleotide
  source 1..20: mol_type = other DNA
cttatctcttcgcagcgagg

SEQ ID NO: 9 - DNA, 20 bp, synthetic construct
  misc_feature 1..20: note = Description of Artificial Sequence: Synthetic oligonucleotide
  source 1..20: mol_type = other DNA
cacacattactccaacattg

SEQ ID NO: 10 - DNA, 20 bp, synthetic construct
  misc_feature 1..20: note = Description of Artificial Sequence: Synthetic oligonucleotide
  source 1..20: mol_type = other DNA
ttccgcaaaatagagcccca

SEQ ID NO: 11 - DNA, 20 bp, synthetic construct
  misc_feature 1..20: note = Description of Artificial Sequence: Synthetic oligonucleotide
  source 1..20: mol_type = other DNA
tgcacagaactatcgtacca

SEQ ID NO: 12 - DNA, 20 bp, synthetic construct
  misc_feature 1..20: note = Description of Artificial Sequence: Synthetic oligonucleotide
  source 1..20: mol_type = other DNA
gcaataagactctttaaaga

SEQ ID NO: 13 - DNA, 20 bp, synthetic construct
  misc_feature 1..20: note = Description of Artificial Sequence: Synthetic oligonucleotide
  source 1..20: mol_type = other DNA
caaagagattacgaatgcct

SEQ ID NO: 14 - DNA, 20 bp, synthetic construct
  misc_feature 1..20: note = Description of Artificial Sequence: Synthetic oligonucleotide
  source 1..20: mol_type = other DNA
caaggcaccccaggtttcca

SEQ ID NO: 15 - DNA, 20 bp, synthetic construct
  misc_feature 1..20: note = Description of Artificial Sequence: Synthetic oligonucleotide
  source 1..20: mol_type = other DNA
ttacgaatgccttggaaacc

SEQ ID NO: 16 - DNA, 20 bp, synthetic construct
  misc_feature 1..20: note = Description of Artificial Sequence: Synthetic oligonucleotide
  source 1..20: mol_type = other DNA
cagagacgcatctgaccctc

SEQ ID NO: 17 - DNA, 20 bp, synthetic construct
  misc_feature 1..20: note = Description of Artificial Sequence: Synthetic oligonucleotide
  source 1..20: mol_type = other DNA
catgcagttctcacacactg

SEQ ID NO: 18 - DNA, 20 bp, synthetic construct
  misc_feature 1..20: note = Description of Artificial Sequence: Synthetic oligonucleotide
  source 1..20: mol_type = other DNA
gtgtgagaactgcatggaga

SEQ ID NO: 19 - DNA, 20 bp, synthetic construct
  misc_feature 1..20: note = Description of Artificial Sequence: Synthetic oligonucleotide
  source 1..20: mol_type = other DNA
tctcatttcaggaaaccact

SEQ ID NO: 20 - DNA, 20 bp, synthetic construct
  misc_feature 1..20: note = Description of Artificial Sequence: Synthetic oligonucleotide
  source 1..20: mol_type = other DNA
agtcatacaccttaaccaag

SEQ ID NO: 21 - DNA, 20 bp, synthetic construct
  misc_feature 1..20: note = Description of Artificial Sequence: Synthetic oligonucleotide
  source 1..20: mol_type = other DNA
ttcaaggaaaccagttgagg

SEQ ID NO: 22 - DNA, 20 bp, synthetic construct
  misc_feature 1..20: note = Description of Artificial Sequence: Synthetic oligonucleotide
  source 1..20: mol_type = other DNA
gagccttgcctggaaatctg

SEQ ID NO: 23 - DNA, 20 bp, synthetic construct
  misc_feature 1..20: note = Description of Artificial Sequence: Synthetic oligonucleotide
  source 1..20: mol_type = other DNA
aagcgtcaaaagtctgccag

SEQ ID NO: 24 - DNA, 20 bp, synthetic construct
  misc_feature 1..20: note = Description of Artificial Sequence: Synthetic oligonucleotide
  source 1..20: mol_type = other DNA
cgttccaactcgaagtgcca

SEQ ID NO: 25 - DNA, 20 bp, synthetic construct
  misc_feature 1..20: note = Description of Artificial Sequence: Synthetic oligonucleotide
  source 1..20: mol_type = other DNA
gagcgactgggacacggtga

SEQ ID NO: 26 - DNA, 20 bp, synthetic construct
  misc_feature 1..20: note = Description of Artificial Sequence: Synthetic oligonucleotide
  source 1..20: mol_type = other DNA
gctgcgcaagaagggcccta

SEQ ID NO: 27 - DNA, 20 bp, synthetic construct
  misc_feature 1..20: note = Description of Artificial Sequence: Synthetic oligonucleotide
  source 1..20: mol_type = other DNA
ttgttctggccagcagcccc

SEQ ID NO: 28 - DNA, 20 bp, synthetic construct
  misc_feature 1..20: note = Description of Artificial Sequence: Synthetic oligonucleotide
  source 1..20: mol_type = other DNA
cttccagagccacatcatcg

SEQ ID NO: 29 - DNA, 20 bp, synthetic construct
  misc_feature 1..20: note = Description of Artificial Sequence: Synthetic oligonucleotide
  source 1..20: mol_type = other DNA
gggactcaccagagagaggt

SEQ ID NO: 30 - DNA, 20 bp, synthetic construct
  misc_feature 1..20: note = Description of Artificial Sequence: Synthetic oligonucleotide
  source 1..20: mol_type = other DNA
cggtcgaaatagaagcccta

SEQ ID NO: 31 - DNA, 20 bp, synthetic construct
  misc_feature 1..20: note = Description of Artificial Sequence: Synthetic oligonucleotide
  source 1..20: mol_type = other DNA
aaaaggatattgtgcaactg

SEQ ID NO: 32 - DNA, 20 bp, synthetic construct
  misc_feature 1..20: note = Description of Artificial Sequence: Synthetic oligonucleotide
  source 1..20: mol_type = other DNA
tgtgcatatttattacatcg

SEQ ID NO: 33 - DNA, 20 bp, synthetic construct
  misc_feature 1..20: note = Description of Artificial Sequence: Synthetic oligonucleotide
  source 1..20: mol_type = other DNA
tttgtgaagatcttgaccaa

SEQ ID NO: 34 - DNA, 20 bp, synthetic construct
  misc_feature 1..20: note = Description of Artificial Sequence: Synthetic oligonucleotide
  source 1..20: mol_type = other DNA
tgtcatgctgaaccgcattg

SEQ ID NO: 35 - DNA, 20 bp, synthetic construct
  misc_feature 1..20: note = Description of Artificial Sequence: Synthetic oligonucleotide
  source 1..20: mol_type = other DNA
ccactctatgaggatagtca

SEQ ID NO: 36 - DNA, 20 bp, synthetic construct
  misc_feature 1..20: note = Description of Artificial Sequence: Synthetic oligonucleotide
  source 1..20: mol_type = other DNA
ttgacatagaagaggcacaa

SEQ ID NO: 37 - DNA, 20 bp, synthetic construct
  misc_feature 1..20: note = Description of Artificial Sequence: Synthetic oligonucleotide
  source 1..20: mol_type = other DNA
gagtactacactcagcagca

SEQ ID NO: 38 - DNA, 20 bp, synthetic construct
  misc_feature 1..20: note = Description of Artificial Sequence: Synthetic oligonucleotide
  source 1..20: mol_type = other DNA
tcacgcacaagaaacgtcca

SEQ ID NO: 39 - DNA, 20 bp, synthetic construct
  misc_feature 1..20: note = Description of Artificial Sequence: Synthetic oligonucleotide
  source 1..20: mol_type = other DNA
aggtctcggtgaaaccacct

SEQ ID NO: 40 - DNA, 20 bp, synthetic construct
  misc_feature 1..20: note = Description of Artificial Sequence: Synthetic oligonucleotide
  source 1..20: mol_type = other DNA
agcattatccaaagagtccg

SEQ ID NO: 41 - DNA, 20 bp, synthetic construct
  misc_feature 1..20: note = Description of Artificial Sequence: Synthetic oligonucleotide
  source 1..20: mol_type = other DNA
atattaattcttaccagtgg

SEQ ID NO: 42 - DNA, 20 bp, synthetic construct
  misc_feature 1..20: note = Description of Artificial Sequence: Synthetic oligonucleotide
  source 1..20: mol_type = other DNA
agctttaaatcaaggttcat

SEQ ID NO: 43 - DNA, 20 bp, synthetic construct
  misc_feature 1..20: note = Description of Artificial Sequence: Synthetic oligonucleotide
  source 1..20: mol_type = other DNA
atcccgagccctaaggtgca

SEQ ID NO: 44 - DNA, 20 bp, synthetic construct
  misc_feature 1..20: note = Description of Artificial Sequence: Synthetic oligonucleotide
  source 1..20: mol_type = other DNA
ggcagcgcggaggacagcgt

SEQ ID NO: 45 - DNA, 20 bp, synthetic construct
  misc_feature 1..20: note = Description of Artificial Sequence: Synthetic oligonucleotide
  source 1..20: mol_type = other DNA
ctcagggggctactaccacc

SEQ ID NO: 46 - DNA, 20 bp, synthetic construct
  misc_feature 1..20: note = Description of Artificial Sequence: Synthetic oligonucleotide
  source 1..20: mol_type = other DNA
gtcaccgacgagaccagaag

SEQ ID NO: 47 - DNA, 20 bp, synthetic construct
  misc_feature 1..20: note = Description of Artificial Sequence: Synthetic oligonucleotide
  source 1..20: mol_type = other DNA
gtcgtggacttcgtactgct

SEQ ID NO: 48 - DNA, 20 bp, synthetic construct
  misc_feature 1..20: note = Description of Artificial Sequence: Synthetic oligonucleotide
  source 1..20: mol_type = other DNA
taatttttaggcaagtgtcg

SEQ ID NO: 49 - DNA, 20 bp, synthetic construct
  misc_feature 1..20: note = Description of Artificial Sequence: Synthetic oligonucleotide
  source 1..20: mol_type = other DNA
ttagctgttagacttgaata

SEQ ID NO: 50 - DNA, 20 bp, synthetic construct
  misc_feature 1..20: note = Description of Artificial Sequence: Synthetic oligonucleotide
  source 1..20: mol_type = other DNA
cgagagccgtcaacttgcgt

SEQ ID NO: 51 - DNA, 20 bp, synthetic construct
  misc_feature 1..20: note = Description of Artificial Sequence: Synthetic oligonucleotide
  source 1..20: mol_type = other DNA
cggcttcaactgcaaaggtg

SEQ ID NO: 52 - DNA, 20 bp, synthetic construct
  misc_feature 1..20: note = Description of Artificial Sequence: Synthetic oligonucleotide
  source 1..20: mol_type = other DNA
tatgaaaaagcagagcgact

SEQ ID NO: 53 - DNA, 20 bp, synthetic construct
  misc_feature 1..20: note = Description of Artificial Sequence: Synthetic oligonucleotide
  source 1..20: mol_type = other DNA
tctggcgggcgagctcacgc

SEQ ID NO: 54 - DNA, 20 bp, synthetic construct
  misc_feature 1..20: note = Description of Artificial Sequence: Synthetic oligonucleotide
  source 1..20: mol_type = other DNA
ctcacgctggttaccgccta

SEQ ID NO: 55 - DNA, 20 bp, synthetic construct
  misc_feature 1..20: note = Description of Artificial Sequence: Synthetic oligonucleotide
  source 1..20: mol_type = other DNA
aaagattacgaacttccctg

SEQ ID NO: 56 - DNA, 20 bp, synthetic construct
  misc_feature 1..20: note = Description of Artificial Sequence: Synthetic oligonucleotide
  source 1..20: mol_type = other DNA
gttaaaaacagacatgccta

SEQ ID NO: 57 - DNA, 20 bp, synthetic construct
  misc_feature 1..20: note = Description of Artificial Sequence: Synthetic oligonucleotide
  source 1..20: mol_type = other DNA
atgcctaaggaggttgtacc

SEQ ID NO: 58 - DNA, 20 bp, synthetic construct
  misc_feature 1..20: note = Description of Artificial Sequence: Synthetic oligonucleotide
  source 1..20: mol_type = other DNA
ctccaggtatcccatcgaaa

SEQ ID NO: 59 - DNA, 20 bp, synthetic construct
  misc_feature 1..20: note = Description of Artificial Sequence: Synthetic oligonucleotide
  source 1..20: mol_type = other DNA
caccaaatacgatagatcag

SEQ ID NO: 60 - DNA, 20 bp, synthetic construct
  misc_feature 1..20: note = Description of Artificial Sequence: Synthetic oligonucleotide
  source 1..20: mol_type = other DNA
tggcggcgtgaatggcaaga

SEQ ID NO: 61 - DNA, 20 bp, synthetic construct
  misc_feature 1..20: note = Description of Artificial Sequence: Synthetic oligonucleotide
  source 1..20: mol_type = other DNA
taggatggtagcacacaacc

SEQ ID NO: 62 - DNA, 20 bp, synthetic construct
  misc_feature 1..20: note = Description of Artificial Sequence: Synthetic oligonucleotide
  source 1..20: mol_type = other DNA
cagcagcagagccccgacgg

SEQ ID NO: 63 - DNA, 20 bp, synthetic construct
  misc_feature 1..20: note = Description of Artificial Sequence: Synthetic oligonucleotide
  source 1..20: mol_type = other DNA
cggcgtgcgaacggaatgtg

SEQ ID NO: 64 - DNA, 20 bp, synthetic construct
  misc_feature 1..20: note = Description of Artificial Sequence: Synthetic oligonucleotide
  source 1..20: mol_type = other DNA
tatagacgctgcccgacgtc

SEQ ID NO: 65 - DNA, 20 bp, synthetic construct
  misc_feature 1..20: note = Description of Artificial Sequence: Synthetic oligonucleotide
  source 1..20: mol_type = other DNA
tccaaagaagggtactgtgg

SEQ ID NO: 66 - DNA, 20 bp, synthetic construct
  misc_feature 1..20: note = Description of Artificial Sequence: Synthetic oligonucleotide
  source 1..20: mol_type = other DNA
acagtacccttctttggaat

SEQ ID NO: 67 - DNA, 20 bp, synthetic construct
  misc_feature 1..20: note = Description of Artificial Sequence: Synthetic oligonucleotide
  source 1..20: mol_type = other DNA
gcgacgggcgcatctacgtg

SEQ ID NO: 68 - DNA, 20 bp, synthetic construct
  misc_feature 1..20: note = Description of Artificial Sequence: Synthetic oligonucleotide
  source 1..20: mol_type = other DNA
cccgacctccataagtcctg

SEQ ID NO: 69 - DNA, 20 bp, synthetic construct
  misc_feature 1..20: note = Description of Artificial Sequence: Synthetic oligonucleotide
  source 1..20: mol_type = other DNA
ggggtcctcgaagcgcacga

SEQ ID NO: 70 - DNA, 20 bp, synthetic construct
  misc_feature 1..20: note = Description of Artificial Sequence: Synthetic oligonucleotide
  source 1..20: mol_type = other DNA
tgctctgtttagaagatgac

SEQ ID NO: 71 - DNA, 20 bp, synthetic construct
  misc_feature 1..20: note = Description of Artificial Sequence: Synthetic oligonucleotide
  source 1..20: mol_type = other DNA
atattcttttctagttaaag

SEQ ID NO: 72 - DNA, 20 bp, synthetic construct
  misc_feature 1..20: note = Description of Artificial Sequence: Synthetic oligonucleotide
  source 1..20: mol_type = other DNA
cctgtaaagaaacaaaagac

SEQ ID NO: 73 - DNA, 20 bp, synthetic construct
  misc_feature 1..20: note = Description of Artificial Sequence: Synthetic oligonucleotide
  source 1..20: mol_type = other DNA
tggagaaagacgtaacttcg

SEQ ID NO: 74 - DNA, 20 bp, synthetic construct
  misc_feature 1..20: note = Description of Artificial Sequence: Synthetic oligonucleotide
  source 1..20: mol_type = other DNA
tctgccctgaggtatgcgat

SEQ ID NO: 75 - DNA, 20 bp, synthetic construct
  misc_feature 1..20: note = Description of Artificial Sequence: Synthetic oligonucleotide
  source 1..20: mol_type = other DNA
attccgcttggtgaaaacga

SEQ ID NO: 76 - DNA, 20 bp, synthetic construct
  misc_feature 1..20: note = Description of Artificial Sequence: Synthetic oligonucleotide
  source 1..20: mol_type = other DNA
caggcacaatagaaacaacg

SEQ ID NO: 77 - DNA, 20 bp, synthetic construct
  misc_feature 1..20: note = Description of Artificial Sequence: Synthetic oligonucleotide
  source 1..20: mol_type = other DNA
ccatttgtaatgctgacttg

SEQ ID NO: 78 - DNA, 20 bp, synthetic construct
  misc_feature 1..20: note = Description of Artificial Sequence: Synthetic oligonucleotide
  source 1..20: mol_type = other DNA
ctgggtcacttgtgccgtgg

SEQ ID NO: 79 - DNA, 20 bp, synthetic construct
  misc_feature 1..20: note = Description of Artificial Sequence: Synthetic oligonucleotide
  source 1..20: mol_type = other DNA
gtcagggttctggatatctg

SEQ ID NO: 80 - DNA, 20 bp, synthetic construct
  misc_feature 1..20: note = Description of Artificial Sequence: Synthetic oligonucleotide
  source 1..20: mol_type = other DNA
tggatttagagtctctcagc

SEQ ID NO: 81 - DNA, 20 bp, synthetic construct
  misc_feature 1..20: note = Description of Artificial Sequence: Synthetic oligonucleotide
  source 1..20: mol_type = other DNA
ctgcggctgtggtccagctg

SEQ ID NO: 82 - DNA, 20 bp, synthetic construct
  misc_feature 1..20: note = Description of Artificial Sequence: Synthetic oligonucleotide
  source 1..20: mol_type = other DNA
acaaaactgtgctagacatg

SEQ ID NO: 83 - DNA, 20 bp, synthetic construct
  misc_feature 1..20: note = Description of Artificial Sequence: Synthetic oligonucleotide
  source 1..20: mol_type = other DNA
ttcttccccagcccaggtaa

SEQ ID NO: 84 - DNA, 20 bp, synthetic construct
  misc_feature 1..20: note = Description of Artificial Sequence: Synthetic oligonucleotide
  source 1..20: mol_type = other DNA
cgtcatgagcagattaaacc

SEQ ID NO: 85 - DNA, 20 bp, synthetic construct
  misc_feature 1..20: note = Description of Artificial Sequence: Synthetic oligonucleotide
  source 1..20: mol_type = other DNA
gagagcgcctgcgacccgag

SEQ ID NO: 86 - DNA, 20 bp, synthetic construct
  misc_feature 1..20: note = Description of Artificial Sequence: Synthetic oligonucleotide
  source 1..20: mol_type = other DNA
ccagcgggtgaagtacacca

SEQ ID NO: 87 - DNA, 20 bp, synthetic construct
  misc_feature 1..20: note = Description of Artificial Sequence: Synthetic oligonucleotide
  source 1..20: mol_type = other DNA
ggagcgcttttcgccgccag

SEQ ID NO: 88 - DNA, 20 bp, synthetic construct
  misc_feature 1..20: note = Description of Artificial Sequence: Synthetic oligonucleotide
  source 1..20: mol_type = other DNA
tgaggcctggaccttatgca

SEQ ID NO: 89 - DNA, 20 bp, synthetic construct
  misc_feature 1..20: note = Description of Artificial Sequence: Synthetic oligonucleotide
  source 1..20: mol_type = other DNA
cctggtggagtgaaccatga

SEQ ID NO: 90 - DNA, 20 bp, synthetic construct
  misc_feature 1..20: note = Description of Artificial Sequence: Synthetic oligonucleotide
  source 1..20: mol_type = other DNA
caagcacttaggttcccctg

SEQ ID NO: 91 - DNA, 20 bp, synthetic construct
  misc_feature 1..20: note = Description of Artificial Sequence: Synthetic oligonucleotide
  source 1..20: mol_type = other DNA
ggtctccctacaattcagcg

SEQ ID NO: 92 - DNA, 20 bp, synthetic construct
  misc_feature 1..20: note = Description of Artificial Sequence: Synthetic oligonucleotide
  source 1..20: mol_type = other DNA
cacagcgcgtgactgcaatg

SEQ ID NO: 93 - DNA, 20 bp, synthetic construct
  misc_feature 1..20: note = Description of Artificial Sequence: Synthetic oligonucleotide
  source 1..20: mol_type = other DNA
tctggggcaccaattctagg

SEQ ID NO: 94 - DNA, 20 bp, synthetic construct
  misc_feature 1..20: note = Description of Artificial Sequence: Synthetic oligonucleotide
  source 1..20: mol_type = other DNA
gagccatgcttggcttacga

SEQ ID NO: 95 - DNA, 20 bp, synthetic construct
  misc_feature 1..20: note = Description of Artificial Sequence: Synthetic oligonucleotide
  source 1..20: mol_type = other DNA
gtacaagtacttatctcatg

SEQ ID NO: 96 - DNA, 20 bp, synthetic construct
  misc_feature 1..20: note = Description of Artificial Sequence: Synthetic oligonucleotide
  source 1..20: mol_type = other DNA
gagataacaacataacaaca

SEQ ID NO: 97 - DNA, 20 bp, synthetic construct
  misc_feature 1..20: note = Description of Artificial Sequence: Synthetic oligonucleotide
  source 1..20: mol_type = other DNA
catattccatagtctttggg

SEQ ID NO: 98 - DNA, 20 bp, synthetic construct
  misc_feature 1..20: note = Description of Artificial Sequence: Synthetic oligonucleotide
  source 1..20: mol_type = other DNA
ctgccccttagcaacttagg

SEQ ID NO: 99 - DNA, 20 bp, synthetic construct
  misc_feature 1..20: note = Description of Artificial Sequence: Synthetic oligonucleotide
  source 1..20: mol_type = other DNA
tgtttaaaaatatgttgaca

SEQ ID NO: 100 - DNA, 20 bp, synthetic construct
  misc_feature 1..20: note = Description of Artificial Sequence: Synthetic oligonucleotide
  source 1..20: mol_type = other DNA
ccaggaatggaaactcacgc

SEQ ID NO: 101 - DNA, 20 bp, synthetic construct
  misc_feature 1..20: note = Description of Artificial Sequence: Synthetic oligonucleotide
  source 1..20: mol_type = other DNA
gaggccgctgaattaacccg

SEQ ID NO: 102 - DNA, 20 bp, synthetic construct
  misc_feature 1..20: note = Description of Artificial Sequence: Synthetic oligonucleotide
  source 1..20: mol_type = other DNA
atacacgcacacttgcagaa

SEQ ID NO: 103 - DNA, 20 bp, synthetic construct
  misc_feature 1..20: note = Description of Artificial Sequence: Synthetic oligonucleotide
  source 1..20: mol_type = other DNA
gagcagacagaaacccaggg

SEQ ID NO: 104 - DNA, 20 bp, synthetic construct
  misc_feature 1..20: note = Description of Artificial Sequence: Synthetic oligonucleotide
  source 1..20: mol_type = other DNA
tgagtctccaaacagaacag

SEQ ID NO: 105 - DNA, 20 bp, synthetic construct
  misc_feature 1..20: note = Description of Artificial Sequence: Synthetic oligonucleotide
  source 1..20: mol_type = other DNA
taatatcactgacttcacgg

SEQ ID NO: 106 - DNA, 20 bp, synthetic construct
  misc_feature 1..20: note = Description of Artificial Sequence: Synthetic oligonucleotide
  source 1..20: mol_type = other DNA
tacacacaatgtaagcagca

SEQ ID NO: 107 - DNA, 20 bp, synthetic construct
  misc_feature 1..20: note = Description of Artificial Sequence: Synthetic oligonucleotide
  source 1..20: mol_type = other DNA
gggagctcaattcgaaacca

SEQ ID NO: 108 - DNA, 20 bp, synthetic construct
  misc_feature 1..20: note = Description of Artificial Sequence: Synthetic oligonucleotide
  source 1..20: mol_type = other DNA
ttggacaggtgagacagtcg

SEQ ID NO: 109 - DNA, 20 bp, synthetic construct
  misc_feature 1..20: note = Description of Artificial Sequence: Synthetic oligonucleotide
  source 1..20: mol_type = other DNA
aagctcactcagatagtgtg

SEQ ID NO: 110 - DNA, 20 bp, synthetic construct
  misc_feature 1..20: note = Description of Artificial Sequence: Synthetic oligonucleotide
  source 1..20: mol_type = other DNA
caggagaaccaccttacacg

SEQ ID NO: 111 - DNA, 20 bp, synthetic construct
  misc_feature 1..20: note = Description of Artificial Sequence: Synthetic oligonucleotide
  source 1..20: mol_type = other DNA
ggacagaccctgattcacaa

SEQ ID NO: 112 - DNA, 20 bp, synthetic construct
  misc_feature 1..20: note = Description of Artificial Sequence: Synthetic oligonucleotide
  source 1..20: mol_type = other DNA
acatggcagtctatgaacag

SEQ ID NO: 113 - DNA, 20 bp, synthetic construct
  misc_feature 1..20: note = Description of Artificial Sequence: Synthetic oligonucleotide
  source 1..20: mol_type = other DNA
cctatagagagtactacttg

SEQ ID NO: 114 - DNA, 20 bp, synthetic construct
  misc_feature 1..20: note = Description of Artificial Sequence: Synthetic oligonucleotide
  source 1..20: mol_type = other DNA
ccaaccgggtcttcattacg

SEQ ID NO: 115 - DNA, 20 bp, synthetic construct
  misc_feature 1..20: note = Description of Artificial Sequence: Synthetic oligonucleotide
  source 1..20: mol_type = other DNA
tcaagcgtagagttccgagt

SEQ ID NO: 116 - DNA, 20 bp, synthetic construct
  misc_feature 1..20: note = Description of Artificial Sequence: Synthetic oligonucleotide
  source 1..20: mol_type = other DNA
tcatgcaattatggacccag

SEQ ID NO: 117 - DNA, 20 bp, synthetic construct
  misc_feature 1..20: note = Description of Artificial Sequence: Synthetic oligonucleotide
  source 1..20: mol_type = other DNA
cgggaaagtgactggccatg

SEQ ID NO: 118 - DNA, 20 bp, synthetic construct
  misc_feature 1..20: note = Description of Artificial Sequence: Synthetic oligonucleotide
  source 1..20: mol_type = other DNA
tgagattgaaatcaaatcgg

SEQ ID NO: 119 - DNA, 20 bp, synthetic construct
  misc_feature 1..20: note = Description of Artificial Sequence: Synthetic oligonucleotide
  source 1..20: mol_type = other DNA
tatgcaatattcatcacgcg

SEQ ID NO: 120 - DNA, 20 bp, synthetic construct
  misc_feature 1..20: note = Description of Artificial Sequence: Synthetic oligonucleotide
  source 1..20: mol_type = other DNA
aatgtgttaaatcaaatgca

SEQ ID NO: 121 - protein, 7 aa, synthetic construct
  REGION 1..7: note = Description of Artificial Sequence: Synthetic peptide
ENLYTQS

SEQ ID NO: 122 - protein, 5 aa, synthetic construct
  REGION 1..5: note = Description of Artificial Sequence: Synthetic peptide
DDDDK

SEQ ID NO: 123 - protein, 4 aa, synthetic construct
  REGION 1..4: note = Description of Artificial Sequence: Synthetic peptide
LVPR

SEQ ID NO: 124 - protein, 4 aa, unidentified
  REGION 1..4: note = Description of Unknown: Hinge polypeptide sequence
CPPC

SEQ ID NO: 125 - DNA, 103 bp, synthetic construct
  misc_feature 1..103: note = Description of Artificial Sequence: Synthetic polynucleotide
  source 1..103: mol_type = other DNA
ggggccactagggacaggatgttttagagctagaaatagcaagttaaaataaggctagtccgttatcaacttgaaaaagtggcaccgagtcggtgcttttttt

SEQ ID NO: 126 - DNA, 20 bp, Homo sapiens
  source 1..20: mol_type = other DNA
ggggccactagggacaggat

SEQ ID NO: 127 - DNA, 23 bp, synthetic construct
  misc_feature 1..23: note = Description of Artificial Sequence: Synthetic oligonucleotide
  source 1..23: mol_type = other DNA
gagccatgcttggcttacgangg

SEQ ID NO: 128 - DNA, 23 bp, Homo sapiens
  source 1..23: mol_type = other DNA
gagccatgcttggcttacgaggg

SEQ ID NO: 129 - DNA, 23 bp, Homo sapiens
  source 1..23: mol_type = other DNA
aatccatgtttcgctttccatag

SEQ ID NO: 130 - DNA, 23 bp, Homo sapiens
  source 1..23: mol_type = other DNA
cagccatgattgggctcctatgc

SEQ ID NO: 131 - DNA, 23 bp, Homo sapiens
  source 1..23: mol_type = other DNA
gatgaatgattggcttcagatgg

SEQ ID NO: 132 - DNA, 23 bp, Homo sapiens
  source 1..23: mol_type = other DNA
gtgccttgctagttttaagatgg

SEQ ID NO: 133 - DNA, 23 bp, Homo sapiens
  source 1..23: mol_type = other DNA
gaaccctgctgggctcaggttag

SEQ ID NO: 134 - DNA, 23 bp, Homo sapiens
  source 1..23: mol_type = other DNA
gtgcctcccttggctgaagaggc

SEQ ID NO: 135 - DNA, 23 bp, Homo sapiens
  source 1..23: mol_type = other DNA
gagccatgattgtgccaccactg

SEQ ID NO: 136 - DNA, 23 bp, Homo sapiens
  source 1..23: mol_type = other DNA
cctccatgcttggcttggcatga

SEQ ID NO: 137 - DNA, 23 bp, Homo sapiens
  source 1..23: mol_type = other DNA
gagtaatacttggctcagcagtg

SEQ ID NO: 138 - DNA, 23 bp, Homo sapiens
  source 1..23: mol_type = other DNA
gagccagggagggcttcccagag

SEQ ID NO: 139 - DNA, 23 bp, Homo sapiens
  source 1..23: mol_type = other DNA
gtgacattctttacttaccacag

SEQ ID NO: 140 - DNA, 23 bp, Homo sapiens
  source 1..23: mol_type = other DNA
gagccaggcttgggctcaggtgc

SEQ ID NO: 141 - DNA, 23 bp, Homo sapiens
  source 1..23: mol_type = other DNA
gagccagtgttgggagacgattg

SEQ ID NO: 142 - DNA, 23 bp, Homo sapiens
  source 1..23: mol_type = other DNA
aagccagatttggatttcgaagt

SEQ ID NO: 143 - DNA, 23 bp, Homo sapiens
  source 1..23: mol_type = other DNA
cagccatgctctgcatatgcagt

SEQ ID NO: 144 - DNA, 23 bp, Homo sapiens
  source 1..23: mol_type = other DNA
gaggcaggtttgccttaagcagt

SEQ ID NO: 145 - DNA, 23 bp, Homo sapiens
  source 1..23: mol_type = other DNA
gacacatgccttgctaactaaag

SEQ ID NO: 146 - DNA, 23 bp, Homo sapiens
  source 1..23: mol_type = other DNA
cagcagtgatgggctcacgacgg